(19) United States
(12) Patent Application Publication
(10) Pub. No.: US 2024/0301385 A1
(43) Pub. Date: Sep. 12, 2024

(54) DNA MODIFYING ENZYMES AND ACTIVE FRAGMENTS AND VARIANTS THEREOF AND METHODS OF USE

(71) Applicant: LIFEEDIT THERAPEUTICS, INC., Durham, NC (US)

(72) Inventors: Tyson D. Bowen, Morrisville, NC (US); Alexandra Briner Crawley, Cary, NC (US); Tedd D. Elich, Durham, NC (US)

(73) Assignee: LIFEEDIT THERAPEUTICS, INC., Durham, NC (US)

(21) Appl. No.: 18/551,749

(22) PCT Filed: Mar. 22, 2022
(86) PCT No.: PCT/US2022/021271

§ 371 Date: Sep. 21, 2023

Related U.S. Application Data
(60) Provisional application No. 63/164,273, filed on Mar. 22, 2021.

(51) Int. Cl. C12N 9/78; C12N 9/22; C12N 15/63

(52) U.S. Cl. CPC C12N 9/78; C12N 9/22; C12N 15/63; C07K 2319/09; C12N 2310/20; C12Y 305/04001

(57) ABSTRACT

Compositions and methods comprising deaminase polypeptides for targeted editing of nucleic acids are provided. Compositions comprise deaminase polypeptides. Also provided are fusion proteins comprising a DNA-binding polypeptide and a deaminase of the invention. The fusion proteins include RNA-guided nucleases fused to deaminases, optionally in complex with guide RNAs. Compositions also include nucleic acid molecules encoding the deaminases or the fusion proteins. Vectors and host cells comprising the nucleic acid molecules encoding the deaminases or the fusion proteins are also provided.

### CROSS-REFERENCE TO RELATED APPLICATIONS

This application claims priority to U.S. Provisional Application No. 63/164,273, filed Mar. 22, 2021, which is incorporated by reference herein in its entirety.

### STATEMENT REGARDING THE SEQUENCE LISTING

The Sequence Listing associated with this application is provided in ASCII format in lieu of a paper copy, and is hereby incorporated by reference into the specification. The ASCII copy named L103438_1240WO_0134_1_SL.txt is 1,310,113 bytes in size, was created on Mar. 22, 2022, and is being submitted electronically via EFS-Web.

### FIELD OF THE INVENTION

The present invention relates to the field of molecular biology and gene editing.

### BACKGROUND OF THE INVENTION

Targeted genome editing or modification is rapidly becoming an important tool for basic and applied research. Initial methods involved engineering nucleases such as meganucleases, zinc finger fusion proteins or TALENs, requiring the generation of chimeric nucleases with engineered, programmable, sequence-specific DNA-binding domains specific for each particular target sequence. RNA-guided nucleases (RGNs), such as the Clustered Regularly Interspaced Short Palindromic Repeats (CRISPR)-associated (Cas) proteins of the CRISPR-Cas bacterial system, allow for the targeting of specific sequences by complexing the nucleases with guide RNA that specifically hybridizes with a particular target sequence. Producing target-specific guide RNAs is less costly and more efficient than generating chimeric nucleases for each target sequence. Such RNA-guided nucleases can be used to edit genomes through the introduction of a sequence-specific, double-stranded break that is repaired via error-prone non-homologous end-joining (NHEJ) to introduce a mutation at a specific genomic location.

Additionally, RGNs are useful for targeted DNA editing approaches. Targeted editing of nucleic acid sequences, for example targeted cleavage, to allow for introduction of a specific modification into genomic DNA, enables a highly nuanced approach to studying gene function and gene expression. RGNs may also be used to generate chimeric proteins which use the RNA-guided activity of the RGN in combination with a DNA modifying enzyme, such as a deaminase, for targeted base editing. Targeted editing may be deployed for targeting genetic diseases in humans or for introducing agronomically beneficial mutations in the genomes of crop plants. The development of genome editing tools provides new approaches to gene editing-based mammalian therapeutics and agrobiotechnology.

### BRIEF SUMMARY OF THE INVENTION

Compositions and methods for modifying a target DNA molecule are provided. The compositions find use in modifying a target DNA molecule of interest. Compositions provided comprise deaminase polypeptides. Also provided are fusion proteins comprising a nucleic acid molecule-binding polypeptide (e.g., DNA-binding polypeptide) and a deaminase polypeptide, and ribonucleoprotein complexes comprising a fusion protein comprising an RNA-guided nuclease and a deaminase polypeptide and ribonucleic acids. Compositions provided also include nucleic acid molecules encoding the deaminase polypeptides or the fusion proteins, and vectors and host cells comprising the nucleic acid molecules. The methods disclosed herein are drawn to binding a target sequence of interest within a target DNA molecule of interest and modifying the target DNA molecule of interest.

In a first aspect, the present disclosure provides a polypeptide comprising an amino acid sequence selected from the group consisting of: a) an amino acid sequence having at least 90% sequence identity to any one of SEQ ID NOs: 2 and 7-12; and b) an amino acid sequence having at least 95% sequence identity to SEQ ID NO: 4 or 6; wherein the polypeptide has deaminase activity.

In some embodiments of the above aspect, the polypeptide comprises an amino acid sequence having at least 95% sequence identity to any one of SEQ ID NOs: 2 and 7-12. In some embodiments, the polypeptide comprises an amino acid sequence having 100% sequence identity to any one of SEQ ID NOs: 2, 4, and 6-12. In some embodiments, the polypeptide is isolated.

In another aspect, the present disclosure provides a nucleic acid molecule comprising a polynucleotide encoding a deaminase polypeptide, wherein the deaminase is encoded by a nucleotide sequence selected from the group consisting of: a) a nucleotide sequence having at least 80% sequence identity to any one of SEQ ID NOs: 114-119; b) a nucleotide sequence having at least 95% sequence identity to any one of SEQ ID NOs: 109, 111, and 113; c) a nucleotide sequence encoding an amino acid sequence having at least 90% sequence identity to any one of SEQ ID NOs: 2 and 7-12; and d) a nucleotide sequence encoding an amino acid sequence having at least 95% sequence identity to SEQ ID NO: 4 or 6.

In some embodiments of the above aspect, the deaminase is encoded by a nucleotide sequence that has at least 90% sequence identity to any one of SEQ ID NOs: 114-119. In some embodiments, the deaminase is encoded by a nucleotide sequence that has at least 95% sequence identity to any one of SEQ ID NOs: 114-119. In some embodiments, the deaminase is encoded by a nucleotide sequence that has 100% sequence identity to any one of SEQ ID NOs: 109, 111, and 113-119. In some embodiments, the deaminase polypeptide has an amino acid sequence having at least 95% sequence identity to any one of SEQ ID NOs: 2 and 7-12. In some embodiments, the deaminase polypeptide has an amino acid sequence having 100% sequence identity to any one of SEQ ID NOs: 2, 4, and 6-12.

In some embodiments of the above aspect, the nucleic acid molecule further comprises a heterologous promoter operably linked to the polynucleotide. In some embodiments, the nucleic acid molecule is isolated.

In another aspect, the present disclosure provides a vector comprising a nucleic acid molecule described hereinabove.

In another aspect, the present disclosure provides a cell comprising a nucleic acid molecule or a vector described hereinabove. In some embodiments, the cell is a prokaryotic cell. In some embodiments, the cell is a eukaryotic cell. In some embodiments, the eukaryotic cell is a mammalian cell. In some embodiments, the mammalian cell is a human cell. In some embodiments, the human cell is an immune cell.

In some embodiments, the immune cell is a stem cell. In some embodiments, the stem cell is an induced pluripotent stem cell. In some embodiments, the eukaryotic cell is an insect or avian cell. In some embodiments, the eukaryotic cell is a fungal cell. In some embodiments, the eukaryotic cell is a plant cell.

In another aspect, the present disclosure provides a plant or a seed comprising a plant cell described hereinabove.

In another aspect, the present disclosure provides a pharmaceutical composition comprising a pharmaceutically acceptable carrier and a polypeptide, a nucleic acid molecule, a vector of embodiment 11, or a cell described hereinabove. In some embodiments, the pharmaceutically acceptable carrier is heterologous to the polypeptide or the nucleic acid molecule. In some embodiments, the pharmaceutically acceptable carrier is not naturally-occurring.

In another aspect, the present disclosure provides a method for making a deaminase comprising culturing a cell described hereinabove under conditions in which the deaminase is expressed.

In another aspect, the present disclosure provides a method for making a deaminase comprising introducing into a cell a nucleic acid molecule or vector described hereinabove and culturing the cell under conditions in which the deminase is expressed.

In some embodiments of the above aspects, the method further comprises purifying the deaminase.

In another aspect, the present disclosure provides a fusion protein comprising a DNA-binding polypeptide and a deaminase having an amino acid sequence selected from the group consisting of: a) an amino acid sequence having at least 90% sequence identity to any one of SEQ ID NOs: 2 and 7-12; and b) an amino acid sequence having at least 95% sequence identity to SEQ ID NO: 4 or 6. In some embodiments, the deaminase has at least 95% sequence identity to any one of SEQ ID NOs: 2 and 7-12. In some embodiments, the deaminase has 100% sequence identity to any one of SEQ ID NOs: 2, 4, and 6-12.

In some embodiments of the above aspect, the deaminase is a cytosine deaminase. In some embodiments, the DNA-binding polypeptide is a meganuclease, a zinc finger fusion protein, or a TALEN; or a variant of a meganuclease, a zinc finger fusion protein, or a TALEN, wherein the nuclease activity has been reduced or inhibited.

In some embodiments of the above aspect, the DNA-binding polypeptide is an RNA-guided, DNA-binding polypeptide. In some embodiments, the RNA-guided, DNA-binding polypeptide is an RNA-guided nuclease (RGN) polypeptide. In some embodiments, the RGN is a Type II or Type V CRISPR-Cas polypeptide. In some embodiments, the RGN is an RGN nickase. In some embodiments, the RGN nickase has an inactive RuvC domain. In some embodiments, the RGN is a nuclease-inactive RGN. In some embodiments, the RGN has an amino acid sequence having at least 90% sequence identity to any one of the RGN sequences in Table 1. In some embodiments, the RGN has an amino acid sequence having at least 95% sequence identity to any one of the RGN sequences in Table 1. In some embodiments, the RGN has an amino acid sequence of any one of the RGN sequences in Table 1. In some embodiments, the RGN has an amino acid sequence having at least 90% sequence identity to any one of SEQ ID NOs: 74, 82, 87, 106, and 107. In some embodiments, the RGN has an amino acid sequence having at least 95% sequence identity to any one of SEQ ID NOs: 74, 82, 87, 106, and 107. In some embodiments, the RGN has an amino acid sequence of any one of SEQ ID NOs: 74, 82, 87, 106, and 107.

In some embodiments of the above aspect, the RGN nickase has an amino acid sequence having at least 90% sequence identity to any one of SEQ ID NOs: 75 and 88-98. In some embodiments, the RGN nickase has an amino acid sequence having at least 95% sequence identity to any one of SEQ ID NOs: 75 and 88-98. In some embodiments, the RGN nickase has an amino acid sequence having any one of SEQ ID NOs: 75 and 88-98.

In some embodiments of the above aspect, the fusion protein further comprises at least one nuclear localization signal (NLS). In some embodiments, the deaminase is fused to the amino terminus of the DNA-binding polypeptide. In some embodiments, the deaminase is fused to the carboxyl terminus of the DNA-binding polypeptide. In some embodiments, the fusion protein further comprises a linker sequence between the DNA-binding polypeptide and the deaminase. In some embodiments, the linker sequence has an amino acid sequence set forth as SEQ ID NO: 78 or 79.

In some embodiments of the above aspect, the fusion protein further comprises a uracil stabilizing protein (USP). In some embodiments, the USP has the sequence set forth as SEQ ID NO: 81. In some embodiments, the fusion protein further comprises a linker sequence between the USP and the deaminase or the DNA-binding polypeptide. In some embodiments, the linker sequence between the USP and the deaminase or the DNA-binding polypeptide has an amino acid sequence set forth as SEQ ID NO: 120.

In some embodiments of the above aspect, the fusion protein has an amino acid sequence of any one of SEQ ID NOs: 67, 68, 146, and 147.

In another aspect, the present disclosure provides a nucleic acid molecule comprising a polynucleotide encoding a fusion protein comprising a DNA-binding polypeptide and a deaminase, wherein the deaminase is encoded by a nucleotide sequence selected from the group consisting of: a) a nucleotide sequence having at least 80% sequence identity to any one of SEQ ID NOs: 114-119; b) a nucleotide sequence having at least 95% sequence identity to any one of SEQ ID NOs: 109, 111, and 113; c) a nucleotide sequence encoding an amino acid sequence having at least 90% sequence identity to any one of SEQ ID NOs: 2 and 7-12; and d) a nucleotide sequence encoding an amino acid sequence having at least 95% sequence identity to SEQ ID NO: 4 or 6.

In some embodiments of the above aspect, the deaminase is encoded by a nucleotide sequence has at least 90% sequence identity to any one of SEQ ID NOs: 114-119. In some embodiments, the deaminase is encoded by a nucleotide sequence has at least 95% sequence identity to any one of SEQ ID NOs: 114-119. In some embodiments, the deaminase nucleotide sequence has 100% sequence identity to any one of SEQ ID NOs: 109, 111, and 113-119. In some embodiments, the deaminase nucleotide sequence encodes an amino acid sequence having at least 95% sequence identity to any one of SEQ ID NOs: 2 and 7-12. In some embodiments, the deaminase nucleotide sequence encodes an amino acid sequence having 100% sequence identity to any one of SEQ ID NOs: 2, 4, and 6-12.

In some embodiments of the above aspect, the deaminase is a cytosine deaminase. In some embodiments, the DNA-binding polypeptide is a meganuclease, a zinc finger fusion protein, or a TALEN; or a variant of a meganuclease, a zinc finger fusion protein, or a TALEN, wherein the nuclease activity has been reduced or inhibited.

In some embodiments of the above aspect, the DNA-binding polypeptide is an RNA-guided, DNA-binding polypeptide. In some embodiments, the RNA-guided, DNA-binding polypeptide is an RNA-guided nuclease (RGN) polypeptide. In some embodiments, the RGN is a Type II or Type V CRISPR-Cas polypeptide. In some embodiments, the RGN is an RGN nickase. In some embodiments, the RGN nickase has an inactive RuvC domain. In some embodiments, the RGN is a nuclease-inactive RGN.

In some embodiments of the above aspect, the RGN has an amino acid sequence having at least 90% sequence identity to any one of the RGN sequences in Table 1. In some embodiments, the RGN has an amino acid sequence having at least 95% sequence identity to any one of the RGN sequences in Table 1. In some embodiments, the RGN has an amino acid sequence of any one of the RGN sequences in Table 1. In some embodiments, the RGN has an amino acid sequence having at least 90% sequence identity to any one of SEQ ID NOs: 74, 82, 87, 106, and 107. In some embodiments, the RGN has an amino acid sequence having at least 95% sequence identity to any one of SEQ ID NOs: 74, 82, 87, 106, and 107. In some embodiments, the RGN has an amino acid sequence of any one of SEQ ID NOs: 74, 82, 87, 106, and 107.

In some embodiments of the above aspect, the RGN nickase has an amino acid sequence having at least 90% sequence identity to any one of SEQ ID NOs: 75 and 88-98. In some embodiments, the RGN nickase has an amino acid sequence having at least 95% sequence identity to any one of SEQ ID NOs: 75 and 88-98. In some embodiments, the RGN nickase has an amino acid sequence having any one of SEQ ID NOs: 75 and 88-98.

In some embodiments of the above aspect, the polynucleotide encoding the fusion protein is operably linked at its 5′ end to a promoter. In some embodiments, the polynucleotide encoding the fusion protein is operably linked at its 3′ end to a terminator. In some embodiments, the fusion protein comprises one or more nuclear localization signals.

In some embodiments of the above aspect, the fusion protein is codon optimized for expression in a eukaryotic cell. In some embodiments, the fusion protein is codon optimized for expression in a prokaryotic cell. In some embodiments, the deaminase is fused to the amino terminus of the DNA-binding polypeptide. In some embodiments, the deaminase is fused to the carboxyl terminus of the DNA-binding polypeptide.

In some embodiments of the above aspect, the fusion protein further comprises a linker sequence between the DNA-binding polypeptide and the deaminase. In some embodiments, the linker sequence has an amino acid sequence set forth as SEQ ID NO: 78 or 79. In some embodiments, the fusion protein further comprises a uracil stabilizing protein (USP). In some embodiments, the USP has the sequence set forth as SEQ ID NO: 81. In some embodiments, the fusion protein further comprises a linker sequence between the USP and the deaminase or the DNA-binding polypeptide. In some embodiments, the linker sequence between the USP and the deaminase or the DNA-binding polypeptide has an amino acid sequence set forth as SEQ ID NO: 120. In some embodiments, the fusion protein has an amino acid sequence set forth as any one of SEQ ID NOs: 67, 68, 146, and 147.

In another aspect, the present disclosure provides a vector comprising a nucleic acid molecule described hereinabove. In some embodiments, the vector further comprises at least one nucleotide sequence encoding a guide RNA (gRNA) capable of hybridizing to a target sequence. In some embodiments, the gRNA is a single guide RNA. In some embodiments, the gRNA is a dual guide RNA.

In another aspect, the present disclosure provides a cell comprising a fusion protein described hereinabove. In some embodiments, the cell further comprises a guide RNA. In some embodiments, the gRNA is a single guide RNA. In some embodiments, the gRNA is a dual guide RNA.

In another aspect, the present disclosure provides a cell comprising a nucleic acid molecule or a vector described hereinabove.

In some of the embodiments of the above aspects, the cell is a prokaryotic cell. In some embodiments of the above aspects, the cell is a eukaryotic cell. In some embodiments, the eukaryotic cell is a mammalian cell. In some embodiments, the mammalian cell is a human cell. In some embodiments, the human cell is an immune cell. In some embodiments, the immune cell is a stem cell. In some embodiments, the stem cell is an induced pluripotent stem cell. In some embodiments, the eukaryotic cell is an insect or avian cell. In some embodiments, the eukaryotic cell is a fungal cell. In some embodiments, the eukaryotic cell is a plant cell.

In another aspect, the present disclosure provides a plant or a seed comprising a cell described hereinabove.

In another aspect, the present disclosure provides a pharmaceutical composition comprising a pharmaceutically acceptable carrier and a fusion protein, nucleic acid molecule, vector, or a cell described hereinabove.

In another aspect, the present disclosure provides a method for making a fusion protein comprising culturing a cell described hereinabove under conditions in which the fusion protein is expressed.

In another aspect, the present disclosure provides a method for making a fusion protein comprising introducing into a cell a nucleic acid molecule or a vector described hereinabove and culturing the cell under conditions in which the fusion protein is expressed.

In some embodiments of the above aspects, the method further comprises purifying the fusion protein.

In another aspect, the present disclosure provides a method for making an RGN fusion ribonucleoprotein complex, comprising introducing into a cell a nucleic acid molecule described hereinabove and a nucleic acid molecule comprising an expression cassette encoding a guide RNA, or a vector described hereinabove, and culturing the cell under conditions in which the fusion protein and the gRNA are expressed and form an RGN fusion ribonucleoprotein complex. In some embodiments, the method further comprises purifying the RGN fusion ribonucleoprotein complex.

In another aspect, the present disclosure provides a system for modifying a target DNA molecule comprising a target DNA sequence, wherein the system comprises: a) a fusion protein or a nucleotide sequence encoding the fusion protein, wherein the fusion protein comprises an RNA-guided nuclease polypeptide (RGN) and a deaminase, wherein the deaminase has an amino acid sequence selected from the group consisting of: i) an amino acid sequence having at least 90% sequence identity to any one of SEQ ID NOs: 2 and 7-12; and ii) an amino acid sequence having at least 95% sequence identity to SEQ ID NO: 4 or 6; and b) one or more guide RNAs capable of hybridizing to the target DNA sequence or one or more nucleotide sequences encoding the one or more guide RNAs (gRNAs); and wherein the one or more guide RNAs are capable of forming a complex with the fusion protein in order to direct the fusion protein to bind to the target DNA sequence and modify the target DNA molecule.

In some embodiments of the above aspect, the deaminase has an amino acid sequence having at least 95% sequence identity to any one of SEQ ID NOs: 2 and 7-12. In some embodiments, the deaminase has an amino acid sequence having 100% sequence identity to any one of SEQ ID NOs: 2, 4, and 6-12. In some embodiments, at least one of the nucleotide sequence encoding the one or more guide RNAs and the nucleotide sequence encoding the fusion protein is operably linked to a promoter.

In some embodiments of the above aspect, the target DNA sequence is a eukaryotic target DNA sequence. In some embodiments, the target DNA sequence is located adjacent to a protospacer adjacent motif (PAM) that is recognized by the RGN.

In some embodiments of the above aspect, the target DNA molecule is within a cell. In some embodiments, the cell is a eukaryotic cell. In some embodiments, the eukaryotic cell is a plant cell. In some embodiments, the eukaryotic cell is a mammalian cell. In some embodiments, the mammalian cell is a human cell. In some embodiments, the human cell is an immune cell. In some embodiments, the immune cell is a stem cell. In some embodiments, the stem cell is an induced pluripotent stem cell. In some embodiments, the eukaryotic cell is an insect cell. In some embodiments, the cell is a prokaryotic cell.

In some embodiments of the above aspect, the RGN of the fusion protein is a Type II or Type V CRISPR-Cas polypeptide. In some embodiments, the RGN of the fusion protein has an amino acid sequence having at least 90% sequence identity to any one of the RGN sequences in Table 1. In some embodiments, the RGN of the fusion protein has an amino acid sequence having at least 95% sequence identity to any one of the RGN sequences in Table 1. In some embodiments, the RGN of the fusion protein has an amino acid sequence of any one of the RGN sequences in Table 1. In some embodiments, the RGN of the fusion protein has an amino acid sequence having at least 90% sequence identity to any one of SEQ ID NOs: 74, 82, 87, 106, and 107. In some embodiments, the RGN of the fusion protein has an amino acid sequence having at least 95% sequence identity to any one of SEQ ID NOs: 74, 82, 87, 106, and 107. In some embodiments, the RGN of the fusion protein has an amino acid sequence of any one of SEQ ID NOs: 74, 82, 87, 106, and 107.

In some embodiments of the above aspect, the RGN of the fusion protein is an RGN nickase. In some embodiments, the RGN nickase has an inactive RuvC domain. In some embodiments, the RGN nickase has an amino acid sequence having at least 90% sequence identity to any one of SEQ ID NOs: 75 and 88-98. In some embodiments, the RGN nickase has an amino acid sequence having at least 95% sequence identity to any one of SEQ ID NOs: 75 and 88-98. In some embodiments, the RGN nickase is any one of SEQ ID NOs: 75 and 88-98. In some embodiments, the RGN of the fusion protein is a nuclease-inactive RGN.

In some embodiments of the above aspect, the fusion protein comprises one or more nuclear localization signals. In some embodiments, the deaminase is fused to the amino terminus of the DNA-binding polypeptide. In some embodiments, the deaminase is fused to the carboxyl terminus of the DNA-binding polypeptide. In some embodiments, the fusion protein further comprises a linker sequence between the DNA-binding polypeptide and the deaminase. In some embodiments, the linker sequence has an amino acid sequence set forth as SEQ ID NO: 78 or 79.

In some embodiments of the above aspect, the fusion protein further comprises a uracil stabilizing protein (USP). In some embodiments, the USP has the sequence set forth as SEQ ID NO: 81. In some embodiments, the fusion protein further comprises a linker sequence between the USP and the deaminase or the DNA-binding polypeptide. In some embodiments, the linker sequence between the USP and the deaminase or the DNA-binding polypeptide has an amino acid sequence set forth as SEQ ID NO: 120.

In some embodiments of the above aspect, the fusion protein has an amino acid sequence set forth as any one of SEQ ID NOs: 67, 68, 146, and 147. In some embodiments, the fusion protein is codon optimized for expression in a eukaryotic cell. In some embodiments, the nucleotide sequences encoding the one or more guide RNAs and the nucleotide sequence encoding a fusion protein are located on one vector.

In another aspect, the present disclosure provides a ribonucleoprotein complex comprising at least one guide RNA and the fusion protein of a system described hereinabove.

In another aspect, the present disclosure provides a cell comprising a system or ribonucleoprotein complex described hereinabove. In some embodiments, the cell is a prokaryotic cell. In some embodiments, the cell is a eukaryotic cell. In some embodiments, the eukaryotic cell is a mammalian cell. In some embodiments, the mammalian cell is a human cell. In some embodiments, the human cell is an immune cell. In some embodiments, the immune cell is a stem cell. In some embodiments, the stem cell is an induced pluripotent stem cell. In some embodiments, the eukaryotic cell is an insect or avian cell. In some embodiments, the eukaryotic cell is a fungal cell. In some embodiments, the eukaryotic cell is a plant cell.

In another aspect, the present disclosure provides a plant or seed comprising a plant cell described hereinabove.

In another aspect, the present disclosure provides a pharmaceutical composition comprising a pharmaceutically acceptable carrier and a system, a ribonucleoprotein complex, or a cell described hereinabove.

In another aspect, the present disclosure provides a method for modifying a target DNA molecule comprising a target DNA sequence, wherein the method comprises delivering a system or a ribonucleoprotein complex described hereinabove to the target DNA molecule or a cell comprising the target DNA molecule.

In some embodiments of the above aspect, the modified target DNA molecule comprises a C>N mutation of at least one nucleotide within the target DNA molecule, wherein N is A, G, or T. In some embodiments, the modified target DNA molecule comprises an C>T mutation of at least one nucleotide within the target DNA molecule. In some embodiments, the modified target DNA molecule comprises an C>G mutation of at least one nucleotide within the target DNA molecule.

In another aspect, the present disclosure provides a method for modifying a target DNA molecule comprising a target sequence, wherein the method comprises: a) assembling an RGN-deaminase ribonucleotide complex in vitro by combining: i) one or more guide RNAs capable of hybridizing to the target DNA sequence; and ii) a fusion protein comprising an RNA-guided nuclease polypeptide (RGN), and at least one deaminase, wherein the deaminase has an amino acid sequence selected from the group consisting of: I) an amino acid sequence having at least 90% sequence identity to any one of SEQ ID NOs: 2 and 7-12; and II) an amino acid sequence having at least 95% sequence identity to SEQ ID NO: 4 or 6; under conditions suitable for formation of the RGN-deaminase ribonucleotide complex; and b) contacting the target DNA molecule or a cell comprising the target DNA molecule with the in vitro-assembled RGN-deaminase ribonucleotide complex; wherein the one or more guide RNAs hybridize to the target DNA sequence, thereby directing the fusion protein to bind to the target DNA sequence and modification of the target DNA molecule occurs.

In some embodiments of the above aspect, the deaminase has an amino acid sequence having at least 95% sequence identity to any one of SEQ ID NOs: 2 and 7-12. In some embodiments, the deaminase has an amino acid sequence having 100% sequence identity to any one of SEQ ID NOs: 2, 4, and 6-12.

In some embodiments, the modified target DNA molecule comprises a C>N mutation of at least one nucleotide within the target DNA molecule, wherein N is A, G, or T. In some embodiments, the modified target DNA molecule comprises a C>T mutation of at least one nucleotide within the target DNA molecule. In some embodiments, the modified target DNA molecule comprises a C>G mutation of at least one nucleotide within the target DNA molecule.

In some embodiments of the above aspect, the RGN of the fusion protein is a Type II or Type V CRISPR-Cas polypeptide. In some embodiments, the RGN of the fusion protein has an amino acid sequence having at least 90% sequence identity to any one of the RGN sequences in Table 1. In some embodiments, the RGN of the fusion protein has an amino acid sequence having at least 95% sequence identity to any one of the RGN sequences in Table 1. In some embodiments, the RGN of the fusion protein has an amino acid sequence of any one of the RGN sequences in Table 1. In some embodiments, the RGN of the fusion protein has an amino acid sequence having at least 90% sequence identity to any one of SEQ ID NOs: 74, 82, 87, 106, and 107. In some embodiments, the RGN of the fusion protein has an amino acid sequence having at least 95% sequence identity to any one of SEQ ID NOs: 74, 82, 87, 106, and 107. In some embodiments, the RGN of the fusion protein has an amino acid sequence of any one of SEQ ID NOs: 74, 82, 87, 106, and 107.

In some embodiments of the above aspect, the RGN of the fusion protein is an RGN nickase. In some embodiments, the RGN nickase has an inactive RuvC domain. In some embodiments, the RGN nickase has an amino acid sequence having at least 90% sequence identity to any one of SEQ ID NOs: 75 and 88-98. In some embodiments, the RGN nickase has an amino acid sequence having at least 95% sequence identity to any one of SEQ ID NOs: 75 and 88-98. In some embodiments, the RGN nickase is any one of SEQ ID NOs: 75 and 88-98. In some embodiments, the RGN of the fusion protein is a nuclease-inactive RGN.

In some embodiments of the above aspect, the fusion protein comprises one or more nuclear localization signals. In some embodiments, the deaminase is fused to the amino terminus of the DNA-binding polypeptide. In some embodiments, the deaminase is fused to the carboxyl terminus of the DNA-binding polypeptide. In some embodiments, the fusion protein further comprises a linker sequence between the DNA-binding polypeptide and the deaminase. In some embodiments, the linker sequence has an amino acid sequence set forth as SEQ ID NO: 78 or 79.

In some embodiments of the above aspect, the fusion protein further comprises a uracil stabilizing protein (USP). In some embodiments, the USP has the sequence set forth as SEQ ID NO: 81. In some embodiments, the fusion protein further comprises a linker sequence between the USP and the deaminase or the DNA-binding polypeptide. In some embodiments, the linker sequence between the USP and the deaminase or the DNA-binding polypeptide has an amino acid sequence set forth as SEQ ID NO: 120.

In some embodiments of the above aspect, the fusion protein has an amino acid sequence set forth as any one of SEQ ID NOs: 67, 68, 146, and 147. In some embodiments, the target DNA sequence is a eukaryotic target DNA sequence. In some embodiments, the target DNA sequence is located adjacent to a protospacer adjacent motif (PAM).

In some embodiments of the above aspect, the target DNA molecule is within a cell. In some embodiments, the cell is a eukaryotic cell. In some embodiments, the eukaryotic cell is a plant cell. In some embodiments, the eukaryotic cell is a mammalian cell. In some embodiments, the mammalian cell is a human cell. In some embodiments, the human cell is an immune cell. In some embodiments, the immune cell is a stem cell. In some embodiments, the stem cell is an induced pluripotent stem cell. In some embodiments, the eukaryotic cell is an insect cell. In some embodiments, the cell is a prokaryotic cell.

In some embodiments of the above aspect, the method further comprises selecting a cell comprising the modified DNA molecule.

In another aspect, the present disclosure provides a cell comprising a modified target DNA sequence according to a method described hereinabove. In some embodiments, the cell is a eukaryotic cell. In some embodiments, the eukaryotic cell is a mammalian cell. In some embodiments, the mammalian cell is a human cell. In some embodiments, the human cell is an immune cell. In some embodiments, the immune cell is a stem cell. In some embodiments, the stem cell is an induced pluripotent stem cell. In some embodiments, the eukaryotic cell is an insect cell. In some embodiments, the cell is a prokaryotic cell. In some embodiments, the eukaryotic cell is a plant cell.

In another aspect, the present disclosure provides a plant or a seed comprising a cell described hereinabove.

In another aspect, the present disclosure provides a pharmaceutical composition comprising a cell described hereinabove, and a pharmaceutically acceptable carrier.

In another aspect, the present disclosure provides a method for producing a genetically modified cell with a correction in a causal mutation for a genetically inherited disease, the method comprising introducing into the cell: a) a fusion protein or a polynucleotide encoding the fusion protein, wherein the fusion protein comprises an RNA-guided nuclease polypeptide (RGN) and a deaminase, wherein the deaminase has an amino acid sequence selected from the group consisting of: i) an amino acid sequence having at least 90% sequence identity to any one of SEQ ID NOs: 2 and 7-12; and ii) an amino acid sequence having at least 95% sequence identity to SEQ ID NO: 4 or 6; and b) one or more guide RNAs (gRNA) capable of hybridizing to a target DNA sequence, or a polynucleotide encoding the gRNA; whereby the fusion protein and gRNA target to the genomic location of the causal mutation and modify the genomic sequence to remove the causal mutation.

In some embodiments of the above aspect, the polynucleotide encoding the fusion protein is operably linked to a promoter active in the cell. In some embodiments, the polynucleotide encoding the gRNA is operably linked to a promoter active in the cell.

In some embodiments of the above aspect, the deaminase has an amino acid sequence having at least 95% sequence identity to any one of SEQ ID NOs: 2 and 7-12. In some embodiments, the deaminase has an amino acid sequence having 100% sequence identity to any one of SEQ ID NOs: 2, 4, and 6-12. In some embodiments, the RGN of the fusion protein is a Type II or Type V CRISPR-Cas polypeptide.

In some embodiments of the above aspect, the RGN of the fusion protein has an amino acid sequence having at least 90% sequence identity to any one of the RGN sequences in Table 1. In some embodiments, the RGN of the fusion protein has an amino acid sequence having at least 95% sequence identity to any one of the RGN sequences in Table 1. In some embodiments, the RGN of the fusion protein has an amino acid sequence of any one of the RGN sequences in Table 1.

In some embodiments of the above aspect, the RGN of the fusion protein is an RGN nickase. In some embodiments, the RGN nickase has an inactive RuvC domain. In some embodiments, the RGN nickase has an amino acid sequence having at least 90% sequence identity to any one of SEQ ID NOs: 75 and 88-98. In some embodiments, the RGN nickase has an amino acid sequence having at least 95% sequence identity to any one of SEQ ID NOs: 75 and 88-98. In some embodiments, the RGN nickase is any one of SEQ ID NOs: 75 and 88-98. In some embodiments, the RGN of the fusion protein is a nuclease-inactive RGN.

In some embodiments of the above aspect, the fusion protein comprises one or more nuclear localization signals. In some embodiments, the deaminase is fused to the amino terminus of the DNA-binding polypeptide. In some embodiments, the deaminase is fused to the carboxyl terminus of the DNA-binding polypeptide. In some embodiments, the fusion protein further comprises a linker sequence between the DNA-binding polypeptide and the deaminase. In some embodiments, the linker sequence has an amino acid sequence set forth as SEQ ID NO: 78 or 79.

In some embodiments of the above aspect, the fusion protein further comprises a uracil stabilizing protein (USP). In some embodiments, the USP has the sequence set forth as SEQ ID NO: 81. In some embodiments, the fusion protein further comprises a linker sequence between the USP and the deaminase or the DNA-binding polypeptide. In some embodiments, the linker sequence between the USP and the deaminase or the DNA-binding polypeptide has an amino acid sequence set forth as SEQ ID NO: 120.

In some embodiments of the above aspect, the fusion protein has an amino acid sequence set forth as any one of SEQ ID NOs: 67, 68, 146, and 147. In some embodiments, the genome modification comprises introducing a C>T mutation of at least one nucleotide within the target DNA sequence. In some embodiments, the genome modification comprises introducing a C>G mutation of at least one nucleotide within the target DNA sequence.

In some embodiments of the above aspect, the cell is an animal cell. In some embodiments, the animal cell is a mammalian cell. In some embodiments, the cell is derived from a dog, cat, mouse, rat, rabbit, horse, sheep, goat, cow, pig, or human.

In some embodiments of the above aspect, the correction of the causal mutation comprises correcting a nonsense mutation. In some embodiments, the genetically inherited disease is a disease listed in Table 23. In some embodiments, the gRNA further comprises a spacer sequence that targets any one of SEQ ID NOs: 122-144, or the complement thereof.

In another aspect, the present disclosure provides a composition comprising: a) a fusion protein comprising a DNA-binding polypeptide and a cytosine deaminase, or a nucleic acid molecule encoding the fusion protein; and b) a second cytosine deaminase or a nucleic acid molecule encoding the second deaminase, wherein the second deaminase has an amino acid sequence selected from the group consisting of: i) an amino acid sequence having at least 90% sequence identity to any one of SEQ ID NOs: 2 and 7-12; and ii) an amino acid sequence having at least 95% sequence identity to SEQ ID NO: 4 or 6.

In some embodiments of the above aspect, the second cytosine deaminase has at least 95% sequence identity to any one of SEQ ID NOs: 2 and 7-12. In some embodiments, the second cytosine deaminase has 100% sequence identity to any one of SEQ ID NOs: 2, 4, and 6-12.

In some embodiments of the above aspect, the first cytosine deaminase has an amino acid sequence selected from the group consisting of: a) an amino acid sequence having at least 90% sequence identity to any one of SEQ ID NOs: 2 and 7-12; and b) an amino acid sequence having at least 95% sequence identity to SEQ ID NO: 4 or 6. In some embodiments, the first cytosine deaminase has at least 95% sequence identity to any one of SEQ ID NOs: 2 and 7-12. In some embodiments, the first cytosine deaminase has 100% sequence identity to any one of SEQ ID NOs: 2, 4, and 6-12.

In some embodiments of the above aspect, the DNA-binding polypeptide is a meganuclease, a zinc finger fusion protein, or a TALEN; or a variant of a meganuclease, a zinc finger fusion protein, or a TALEN, wherein the nuclease activity has been reduced or inhibited. In some embodiments, the DNA-binding polypeptide is an RNA-guided, DNA-binding polypeptide. In some embodiments, the RNA-guided, DNA-binding polypeptide is an RNA-guided nuclease (RGN) polypeptide.

In some embodiments of the above aspect, the RGN is an RGN nickase. In some embodiments, the RGN is a nuclease-inactive RGN.

In another aspect, the present disclosure provides a vector comprising a nucleic acid molecule encoding a fusion protein and a nucleic acid molecule encoding a second cytosine deaminase, wherein the fusion protein comprises a DNA-binding polypeptide and a first cytosine deaminase, and wherein the second cytosine deaminase has an amino acid sequence selected from the group consisting of: a) an amino acid sequence having at least 90% sequence identity to any one of SEQ ID NOs: 2 and 7-12; and b) an amino acid sequence having at least 95% sequence identity to SEQ ID NO: 4 or 6.

In some embodiments of the above aspect, the second cytosine deaminase has at least 95% sequence identity to any one of SEQ ID NOs: 2 and 7-12. In some embodiments, the second cytosine deaminase has 100% sequence identity to any one of SEQ ID NOs: 2, 4, and 6-12.

In some embodiments of the above aspect, the first cytosine deaminase has an amino acid sequence selected from the group consisting of: a) an amino acid sequence having at least 90% sequence identity to any one of SEQ ID NOs: 2 and 7-12; and b) an amino acid sequence having at least 95% sequence identity to SEQ ID NO: 4 or 6.

In some embodiments of the above aspect, the first cytosine deaminase has at least 95% sequence identity to any one of SEQ ID NOs: 2 and 7-12. In some embodiments, the first cytosine deaminase has 100% sequence identity to any one of SEQ ID NOs: 2, 4, and 6-12. In some embodiments, the DNA-binding polypeptide is a meganuclease, a zinc finger fusion protein, or a TALEN; or a variant of a meganuclease, a zinc finger fusion protein, or a TALEN, wherein the nuclease activity has been reduced or inhibited. In some embodiments, the DNA-binding polypeptide is an RNA-guided, DNA-binding polypeptide. In some embodiments, the RNA-guided, DNA-binding polypeptide is an RNA-guided nuclease (RGN) polypeptide.

In some embodiments of the above aspect, the RGN is an RGN nickase. In some embodiments, the RGN is a nuclease-inactive RGN.

In another aspect, the present disclosure provides a cell comprising a vector described hereinabove.

In another aspect, the present disclosure provides a cell comprising: a) a fusion protein comprising a DNA-binding polypeptide and a first cytosine deaminase; or a nucleic acid molecule encoding the fusion protein; and b) a second cytosine deaminase or a nucleic acid molecule encoding the second cytosine deaminase, wherein the second cytosine deaminase has an amino acid sequence selected from the group consisting of: i) an amino acid sequence having at least 90% sequence identity to any one of SEQ ID NOs: 2 and 7-12; and ii) an amino acid sequence having at least 95% sequence identity to SEQ ID NO: 4 or 6.

In some embodiments of the above aspect, the second cytosine deaminase has at least 95% sequence identity to any one of SEQ ID NOs: 2 and 7-12. In some embodiments, the second cytosine deaminase has 100% sequence identity to any one of SEQ ID NOs: 2, 4, and 6-12.

In some embodiments of the above aspect, the first cytosine deaminase has an amino acid sequence selected from the group consisting of: a) an amino acid sequence having at least 90% sequence identity to any one of SEQ ID NOs: 2 and 7-12; and b) an amino acid sequence having at least 95% sequence identity to SEQ ID NO: 4 or 6. In some embodiments, the first cytosine deaminase has at least 95% sequence identity to any one of SEQ ID NOs: 2 and 7-12. In some embodiments, the first cytosine deaminase has 100% sequence identity to any one of SEQ ID NOs: 2, 4, and 6-12. In some embodiments, the DNA-binding polypeptide is a meganuclease, a zinc finger fusion protein, or a TALEN; or a variant of a meganuclease, a zinc finger fusion protein, or a TALEN, wherein the nuclease activity has been reduced or inhibited. In some embodiments, the DNA-binding polypeptide is an RNA-guided, DNA-binding polypeptide.

In some embodiments of the above aspect, the RNA-guided, DNA-binding polypeptide is an RNA-guided nuclease (RGN) polypeptide. In some embodiments, the RGN is an RGN nickase. In some embodiments, the RGN is a nuclease-inactive RGN.

In another aspect, the present disclosure provides a pharmaceutical composition comprising a pharmaceutically acceptable carrier and a composition, a vector, or a cell described hereinabove.

In another aspect, the present disclosure provides a method for treating a disease, wherein the method comprises administering to a subject in need thereof a fusion protein, a nucleic acid molecule, a vector, a cell, a system, a ribonucleoprotein complex, a composition, or a pharmaceutical composition described herein.

In some embodiments of the above aspect, the disease is associated with a causal mutation and the pharmaceutical composition corrects the causal mutation. In some embodiments, the disease is a disease a disease listed in Table 23.

In another aspect, the present disclosure provides a use of a fusion protein, a nucleic acid molecule, a vector, a cell, a system, a ribonucleoprotein complex, or a composition described herein for the treatment of a disease in a subject.

In some embodiments of the above aspect, the disease is associated with a causal mutation and the treating comprises correcting the causal mutation. In some embodiments, the disease is a disease listed in Table 23.

In another aspect, the present disclosure provides a use of a fusion protein, a nucleic acid molecule, a vector, a cell, a system, a ribonucleoprotein complex, or a composition for the manufacture of a medicament useful for treating a disease. In some embodiments, the disease is associated with a causal mutation and an effective amount of the medicament corrects the causal mutation. In some embodiments, the disease is a disease listed in Table 23.

### DETAILED DESCRIPTION

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

This disclosure provides cytosine deaminases and fusion proteins that comprise a nucleic acid molecule-binding polypeptide, such as a DNA-binding polypeptide, and a deaminase polypeptide. In certain embodiments, the DNA-binding polypeptide is a sequence-specific DNA-binding polypeptide, in that the DNA-binding polypeptide binds to a target sequence at a greater frequency than binding to a randomized background sequence. In some embodiments, the DNA-binding polypeptide is or is derived from a meganuclease, zinc finger fusion protein, or TALEN. In some embodiments, the fusion protein comprises an RNA-guided DNA-binding polypeptide and a deaminase polypeptide. In some embodiments, the RNA-guided DNA-binding polypeptide is an RNA-guided nuclease, such as a CRISPR-Cas (e.g., Cas9) polypeptide that binds to a guide RNA (also referred to as gRNA), which, in turn, binds a target nucleic acid sequence via strand hybridization.

The deaminase polypeptides disclosed herein can deaminate a nucleobase, such as, for example, cytosine. The deamination of a nucleobase by a deaminase can lead to a point mutation at the respective residue, which is referred to herein as “nucleic acid editing”, or “base editing”. Fusion proteins comprising an RNA-guided nuclease (RGN) polypeptide and a deaminase can thus be used for the targeted editing of nucleic acid sequences.

Such fusion proteins are useful for targeted editing of DNA in vitro, e.g., for the generation of genetically modified cells. These genetically modified cells may be plant cells or animal cells. Such fusion proteins may also be useful for the introduction of targeted mutations, e.g., for the correction of genetic defects in mammalian cells ex vivo, e.g., in cells obtained from a subject that are subsequently re-introduced into the same or another subject; and for the introduction of targeted mutations, e.g., the correction of genetic defects or the introduction of deactivating mutations in disease-associated genes in a mammalian subject. Such fusion proteins may also be useful for the introduction of targeted mutations in plant cells, e.g., for the introduction of beneficial or agronomically valuable traits or alleles.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof.

Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

### II. Deaminases

The term “deaminase” refers to an enzyme that catalyzes a deamination reaction. The deaminases of the invention are nucleobase deaminases and the terms “deaminase” and “nucleobase deaminase” are used interchangeably herein. The deaminase may be a naturally-occurring deaminase enzyme or an active fragment or variant thereof. A deaminase may be active on single-stranded nucleic acids, such as ssDNA or ssRNA, or on double-stranded nucleic acids, such as dsDNA or dsRNA. In some embodiments, the deaminase is only capable of deaminating ssDNA and does not act on dsDNA.

The presently disclosed methods and compositions comprise a cytosine deaminase that catalyzes the hydrolytic deamination of cytosine, cytidine, or deoxycytidine to uracil. Cytosine deaminases may work on either DNA or RNA, and typically operate on single-stranded nucleic acid molecules. In further embodiments, the cytosine deaminase is an apolipoprotein B mRNA-editing complex (APOBEC) family deaminase. In some embodiments, the deaminase is an APOBEC1 family deaminase. In some embodiments, the cytosine deaminase is an activation-induced cytidine deaminase (AID). In some embodiments, the deaminase is an ACF1/ASE deaminase. Additional suitable deaminase enzymes will be apparent to the skilled artisan based on this disclosure. A fusion protein comprising a DNA-binding polypeptide and a cytosine deaminase is referred to herein as a “C-base editor”, “cytosine base editor” or “CBE”. CBEs can convert a cytosine to uracil, which can be subsequently converted to thymine through DNA replication or repair. In some embodiments, a CBE converts a cytosine to a guanine or a cytosine to an adenine. Without being bound by any theory or mechanism of action, it is believed that conversion of a cytosine to a guanine or adenine by a cytosine base editor is due to the deamination of the cytosine into a uracil and the subsequent activity of a uracil DNA glycosylase during base excision repair of the uracil residue.

In some embodiments, the presently disclosed cytosine deaminases are used in combination with an adenine deaminase. In some embodiments, the adenine deaminase is an ADAT family deaminase or a variant thereof. Deamination of adenine, adenosine, or deoxyadenosine yields inosine, which is treated as guanine by polymerases. To date there are no known naturally occurring adenine deaminases that deaminate adenine in DNA. Several methods have been employed to evolve and optimize adenine deaminase acting on tRNA (ADAT) proteins to be active on DNA molecules in mammalian cells (Gaudelli et al, 2017; Koblan, L. W. et al, 2018, Nat Biotechnol 36, 843-846; Richter, M. F. et al, 2020, Nat Biotechnol, doi:10.1038/s41587-020-0562-8, each of which are incorporated by reference in their entirety herein). One such method uses a bacterial selection assay where only cells with the ability to activate antibiotic resistance through A:T>G:C conversions are able to survive. In some embodiments, the presently disclosed compositions and methods that comprise a presently disclosed cytosine deaminase further comprise an adenine deaminase set forth in U.S. Provisional Patent Application Nos. 63/077,089, filed Sep. 11, 2020, and 63/146,840, filed Feb. 8, 2021, and PCT International Application No. PCT/US2021/049853, filed Sep. 10, 2021, each of which is herein incorporated by reference in its entirety.

The present invention relates to cytosine deaminase polypeptides identified from bacteria and cytosine deaminases which were produced through the truncation of bacterial deaminases. Cytosine deaminases are presently disclosed and set forth as SEQ ID NOs: 2, 4, and 6-12. The deaminases of the invention may be used for the editing of DNA or RNA molecules. In some embodiments, the deaminases of the invention may be used for editing of ssDNA or ssRNA molecules. The cytosine deaminases described herein are useful as deaminases alone or as components in fusion proteins. A fusion protein comprising a DNA-targeting polypeptide and a cytosine deaminase polypeptide is referred to herein as a “C-based editor”, “cytosine base editor”, or a “CBE” and can be used for the targeted editing of nucleic acid sequences.

“Base editors” are fusion proteins comprising a DNA-targeting polypeptide, such as an RGN, and a deaminase. Cytosine base editors (CBEs) comprise a DNA-targeting protein, such as an RGN, and a cytosine deaminase. CBEs function through the deamination of cytosine into uracil on a DNA target molecule. Uracil is then subsequently converted to thymine through DNA replication or repair. In some embodiments, the presently disclosed cytosine deaminases or active variants or fragments thereof introduce C>N mutations in a DNA molecule, wherein N is A, G, or T. In some embodiments, the presently disclosed cytosine deaminases or fusion proteins comprising the same introduce C>T mutations in a DNA molecule. In some embodiments, the presently disclosed cytosine deaminases or fusion proteins comprising the same introduce C>G mutations in a DNA molecule.

In those embodiments wherein the deaminase has been targeted to a specific region of a nucleic acid molecule via fusion with a DNA-binding polypeptide, the mutation rate of cytosines within or adjacent to the target sequence to which the DNA-binding polypeptide binds can be measured using any method known in the art, including polymerase chain reaction (PCR), restriction fragment length polymorphism (RFLP), or DNA sequencing.

The presently disclosed deaminases or active variants or fragments thereof that retain deaminase activity may be introduced into the cell as part of a deaminase-DNA-binding polypeptide fusion, and/or may be co-expressed with a DNA-binding polypeptide-deaminase fusion, to increase the efficiency of introducing the desired C>N (wherein N is A, T, or G) mutation, such as a C>T or C>G mutation, in a target DNA molecule. The presently disclosed deaminases have the amino acid sequence of any of SEQ ID NOs: 2, 4, and 6-12 or a variant or fragment thereof retaining deaminase activity. In some embodiments, the deaminase has an amino acid sequence having at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to the amino acid sequence of any of SEQ ID NOs: 2, 4, and 6-12. In particular embodiments, the deaminase comprises an amino acid sequence having at least 80% sequence identity to any one of SEQ ID NOs: 2 and 7-12. In certain embodiments, the deaminase comprises an amino acid sequence having at least 95% sequence identity to SEQ ID NO: 4 or 6.

### III. Nucleic Acid Molecule-Binding Polypeptides

Some aspects of this disclosure provide fusion proteins that comprise a nucleic acid molecule-binding polypeptide and a deaminase polypeptide. While binding to and targeted editing of RNA molecules is contemplated by the present invention, in some embodiments, the nucleic acid molecule-binding polypeptide of the fusion protein is a DNA-binding polypeptide. Such fusion proteins are useful for targeted editing of DNA in vitro, ex vivo, or in vivo. These fusion proteins are active in mammalian cells and are useful for targeted editing of DNA molecules.

The term “fusion protein” as used herein refers to a hybrid polypeptide which comprises protein domains from at least two different proteins. A fusion protein may comprise more than one different domain, for example, a DNA-binding domain and a deaminase. In some embodiments, a fusion protein is in a complex with, or is in association with, a nucleic acid, e.g., RNA.

In some embodiments, the presently disclosed fusion proteins comprise a DNA-binding polypeptide. As used herein, the term “DNA-binding polypeptide” refers to any polypeptide which is capable of binding to DNA. In certain embodiments, the DNA-binding polypeptide portion of the presently disclosed fusion proteins binds to double-stranded DNA. In particular embodiments, the DNA-binding polypeptide binds to DNA in a sequence-specific manner. As used herein, the terms “sequence-specific” or “sequence-specific manner” refer to the selective interaction with a specific nucleotide sequence.

Two polynucleotide sequences can be considered to be substantially complementary when the two sequences hybridize to each other under stringent conditions. Likewise, a DNA-binding polypeptide is considered to bind to a particular target sequence in a sequence-specific manner if the DNA-binding polypeptide binds to its sequence under stringent conditions. By “stringent conditions” or “stringent hybridization conditions” is intended conditions under which the two polynucleotide sequences (or the polypeptide binds to its specific target sequence) will bind to each other to a detectably greater degree than to other sequences (e.g., at least 2-fold over background). Stringent conditions are sequence-dependent and will be different in different circumstances. Typically, stringent conditions will be those in which the salt concentration is less than 1.5 M Na ion, typically about 0.01 to 1.0 M Na ion concentration (or other salts) at pH 7.0 to 8.3, and the temperature is at least 30° C. for short sequences (e.g., 10 to 50 nucleotides) and at least 60° C. for long sequences (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The Tm is the temperature (under defined ionic strength and pH) at which 50% of a complementary target sequence hybridizes to a perfectly matched sequence. For DNA-DNA hybrids, the Tm can be approximated from the equation of Meinkoth and Wahl (1984) Anal. Biochem. 138:267-284: Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. An extensive guide to the hybridization of nucleic acids is found in Tijssen (1993) Laboratory Techniques in Biochemistry and Molecular Biology-Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, New York).

In certain embodiments, the sequence-specific DNA-binding polypeptide is an RNA-guided, DNA-binding polypeptide (RGDBP). As used herein, the terms “RNA-guided, DNA-binding polypeptide” and “RGDBP” refer to polypeptides capable of binding to DNA through the hybridization of an associated RNA molecule with the target DNA sequence.

In some embodiments, the DNA-binding polypeptide of the fusion protein is a nuclease, such as a sequence-specific nuclease. As used herein, the term “nuclease” refers to an enzyme that catalyzes the cleavage of phosphodiester bonds between nucleotides in a nucleic acid molecule. In some embodiments, the DNA-binding polypeptide is an endonuclease, which is capable of cleaving phosphodiester bonds between nucleotides within a nucleic acid molecule, whereas in certain embodiments, the DNA-binding polypeptide is an exonuclease that is capable of cleaving the nucleotides at either end (5′ or 3′) of a nucleic acid molecule. In some embodiments, the sequence-specific nuclease is selected from the group consisting of a meganuclease, a zinc finger nuclease, a TAL-effector DNA binding domain-nuclease fusion protein (TALEN), and an RNA-guided nuclease (RGN) or variants thereof wherein the nuclease activity has been reduced or inhibited.

As used herein, the term “meganuclease” or “homing endonuclease” refers to endonucleases that bind a recognition site within double-stranded DNA that is 12 to 40 bp in length. Non-limiting examples of meganucleases are those that belong to the LAGLIDADG family that comprise the conserved amino acid motif LAGLIDADG (SEQ ID NO: 85). The term “meganuclease” can refer to a dimeric or single-chain meganuclease.

As used herein, the term “zinc finger nuclease” or “ZFN” refers to a chimeric protein comprising a zinc finger DNA-binding domain and a nuclease domain.

As used herein, the term “TAL-effector DNA binding domain-nuclease fusion protein” or “TALEN” refers to a chimeric protein comprising a TAL effector DNA-binding domain and a nuclease domain.

In certain embodiments, the DNA-binding polypeptide is one which is capable of generating a single-stranded region within a double-stranded DNA molecule. An example of a single-stranded region is the single-stranded loop comprised within an R-loop, which is a three-stranded nucleic acid structure comprising a region of single-stranded DNA that is formed within a double-stranded DNA molecule that results from the hybridization of the complementary strand to a single-stranded RNA or DNA molecule. A cytosine within or adjacent to the single-stranded region of the R loop can be deaminated by a cytosine deaminase that has activity on single-stranded nucleic acids (e.g., ssDNA). In some of these embodiments, the DNA-binding polypeptide that is capable of generating an R-loop within a double-stranded DNA molecule is an RNA-guided DNA-binding polypeptide or a RGN nuclease. As used herein, the term “RNA-guided nuclease” or “RGN” refers to an RNA-guided, DNA-binding polypeptide that has nuclease activity. RGNs are considered “RNA-guided” because guide RNAs form a complex with the RNA-guided nucleases to direct the RNA-guided nuclease to bind to a target sequence and in some embodiments, introduce a single-stranded or double-stranded break at the target sequence.

In certain embodiments, the RGN is a nickase or nuclease-inactive RGN. The term “RGN polypeptide” encompasses RGN polypeptides that only cleave a single strand of a target nucleotide sequence, which is referred to herein as a nickase. Such RGNs have a single functioning nuclease domain. RGN nickases can be naturally-occurring nickases or can be RGN proteins that naturally cleave both strands of a double-stranded nucleic acid molecule that have been mutated within one or more nuclease domains such that the nuclease activity of these mutated domains is reduced or eliminated, to become a nickase.

In some embodiments, the nickase RGN of the fusion protein comprises a mutation (e.g., a D10A mutation, wherein amino acid numbering is based on the Streptococcus pyogenes Cas9 sequence set forth as SEQ ID NO: 99) which renders the RGN capable of cleaving only the non-base edited, target strand (the strand which comprises the PAM and is base paired to a gRNA) of a nucleic acid duplex. A nickase comprising a D10A mutation, or an equivalent mutation, has an inactivated RuvC nuclease domain and cleaves the targeted strand. D10A nickases are not able to cleave the non-targeted strand of the DNA, i.e., the strand where base editing is desired. In these embodiments, the RGN nicks the target strand, while the complementary, non-target strand is modified by the deaminase. Cellular DNA-repair machinery may repair the nicked, target strand using the modified non-target strand as a template, thereby introducing a mutation in the DNA.

Thus, in some of embodiments, the nickase comprises an inactive RuvC domain. RuvC domains have an RNase H fold structure (see, e.g., Nishimasu et al. (2014) Cell 156(5):935-949, which is incorporated by reference in its entirety). RuvC domains of RGNs are often split RuvC domains, comprising two or more non-adjacent regions within the linear amino acid sequence. For example, the RuvC domain of Streptococcus pyogenes Cas9 comprises amino acid residues 1-59, 718-769 and 909-1098 of SEQ ID NO: 99. A non-limiting example of a mutation within a RuvC domain that inactivates its nuclease activity is the D10A mutation that mutates the first aspartic acid residue in the split RuvC nuclease domain. The present application discloses several D10A nickase variants or homologous nickase variants of described RGNs wherein a RuvC domain is inactivated. nAPG07433.1 and nAPG08290.1 (set forth as SEQ ID NOs: 75 and 88, respectively) are nickase variants of APG07433.1 and APG08290.1, which are set forth as SEQ ID NO: 44 and 87, respectively, and are described in WO 2019/23566 (incorporated by reference in its entirety herein). nAPG07433.1-del and nAPG08290.1-del (set forth as SEQ ID NOs: 97 and 98, respectively) are deletion mutants of APG07433.1 and APG08290.1, respectively, and are described in U.S. Provisional Application Nos. 63/077,089, filed Sep. 11, 2020, and 63/146,840, filed Feb. 8, 2021, and PCT International Application No. PCT/US2021/049853, filed Sep. 10, 2021. nAPG00969 (set for as SEQ ID NO: 89) and nAPG09748 (set forth as SEQ ID NO: 90) are nickase variants of APG00969 and APG09748, respectively, which are described in WO 2020/139783 (incorporated by reference in its entirety herein). nAPG06646 (set forth as SEQ ID NO: 91) and nAPG09882 (set forth as SEQ ID NO: 92) are nickase variants of APG06646 and APG09882, respectively, which are described in PCT Publication No. WO 2021/030344 (incorporated by reference in its entirety herein). nAPG03850, nAPG07553, nAPG055886, and nAPG01604 are set forth as SEQ ID NOs: 93-96, respectively, and are nickase variants of APG03850, APG07553, APG055886, and APG01604 which are described in the pending U.S. Provisional Application Nos. 63/014,970 and 63/077,211 and PCT Publication No. WO 2021/21702 (each of which is incorporated by reference in its entirety herein).

In some embodiments, the nickase RGN of the fusion protein comprises a mutation (e.g., a H840A mutation, wherein amino acid numbering is based on the Streptococcus pyogenes Cas9 sequence set forth as SEQ ID NO: 99), which renders the RGN capable of cleaving only the base-edited, non-target strand (the strand which does not comprise the PAM and is not base paired to a gRNA) of a nucleic acid duplex. In some of these embodiments, the nickase comprises an inactive HNH nuclease domain. The HNH nuclease domain of RGNs have a ββα-metal fold (see, e.g., Nishimasu et al. 2014). The HNH nuclease domain of the Streptococcus pyogenes Cas9, for example, comprises amino acid residues 775-908 of SEQ ID NO: 99. A non-limiting example of a mutation within a HNH domain that inactivates its nuclease activity is the H840A mutation that mutates the first histidine of the HNH nuclease domain. The deaminase with an inactivated HNH domain acts on the non-target strand.

Methods for inactivating a RuvC and/or HNH domain of a RGN are known in the art and generally comprise mutating the first aspartic acid within a split RuvC domain and/or the first histidine of the HNH domain. Typically, the aspartic acid residue or histidine residue is mutated to an alanine. Other amino acid residues within the RuvC domain that can be mutated to inactivate nuclease activity of the domain include Glu762, His983, and Asp986 (typically to an alanine), wherein amino acid numbering is based on the Streptococcus pyogenes Cas9 sequence set forth as SEQ ID NO: 99. Other amino acid residues within the HNH domain that can be mutated include D839 and N863 (typically to an alanine), wherein amino acid numbering is based on the Streptococcus pyogenes Cas9 sequence set forth as SEQ ID NO: 99.

In some embodiments, the RGN of the fusion protein is nuclease dead. As used herein, an RGN protein that has been mutated to become nuclease-inactive or “dead” can be referred to as an RNA-guided, DNA-binding polypeptide or a nuclease-inactive RGN or nuclease-dead RGN. Methods for generating a nuclease-inactive RGN are known in the art and generally comprise mutating the sole nuclease domain or all of the nuclease domains of an RGN to render the nuclease domain(s) inactive. In those embodiments where the RGN only comprises a single nuclease domain (e.g., RuvC domain), the nuclease inactive variant will have at least one mutation within the RuvC domain that results in inactivation of the RuvC nuclease domain. In those embodiments wherein the RGN comprises more than one nuclease domain, such as a RuvC and an HNH domain, at least one mutation within each of the RuvC and the HNH domain renders both nuclease domains inactive.

One exemplary suitable nuclease-inactive RGN is the D10A/H840A Cas9 mutant (see, e.g., Qi et al., Cell. 2013; 152(5): 1173-83, the entire contents of which are incorporated herein by reference). Additionally, suitable nuclease-inactive variants of other known RNA guided nucleases (RGNs) can be determined (for example, a nuclease-inactive variant of the RGN APG08290.1 or RGN APG07433.1 disclosed in U.S. Patent Publication No. 2019/0367949, the entire contents of which are incorporated herein by reference herein, or dAPG09298 set forth as SEQ ID NO: 83).

Other additional exemplary suitable nuclease inactive RGN variants include, but are not limited to, D10A/D839A/H840A, and D10A/D839A/H840A/N863A mutant domains (See, e.g., Mali et al., Nature Biotechnology. 2013; 31(9): 833-838, the entire contents of which are incorporated herein by reference).

Additional suitable RGN proteins mutated to be nickases or inactive nucleases will be apparent to those of skill in the art based on this disclosure and knowledge in the field (such as for example the RGNs disclosed in PCT Publication No. WO 2019/236566, which is herein incorporated by reference in its entirety) and are within the scope of this disclosure.

In some embodiments the RGN nickase retaining nickase activity comprises an amino acid sequence that has at least 60%, at least 65%, at least 70%, at least 75%, at least 80%, at least 85%, at least 90%, at least 95%, at least 96%, at least 97%, at least 98%, at least 99%, or at least 99.5% identity to any one of SEQ ID NOs: 75 and 88-98.

Any method known in the art for introducing mutations into an amino acid sequence, such as PCR-mediated mutagenesis and site-directed mutagenesis, can be used for generating nickases or nuclease-dead RGNs. See, e.g., U.S. Publ. No. 2014/0068797 and U.S. Pat. No. 9,790,490; each of which is incorporated herein by reference in its entirety.

RNA-guided nucleases (RGNs) allow for the targeted manipulation of a single site within a genome and are useful in the context of gene targeting for therapeutic and research applications. In a variety of organisms, including mammals, RNA-guided nucleases have been used for genome engineering by stimulating either non-homologous end joining or homologous recombination. RGNs include CRISPR-Cas proteins, which are RNA-guided nucleases directed to the target sequence by a guide RNA (gRNA) as part of a Clustered Regularly Interspaced Short Palindromic Repeats (CRISPR) RNA-guided nuclease system, or active variants or fragments thereof.

Some aspects of this disclosure provide fusion proteins that comprise an RNA-guided DNA-binding polypeptide and a deaminase polypeptide, specifically a cytosine deaminase polypeptide. In some embodiments, the RNA-guided DNA-binding polypeptide is an RNA-guided nuclease (RGN). In further embodiments, the RNA-guided nuclease is a naturally-occurring CRISPR-Cas protein or an active variant or fragment thereof. CRISPR-Cas systems are classified into Class 1 or Class 2 systems. Class 2 systems comprise a single effector nuclease and include Types II, V, and VI. The Class 1 and 2 systems are subdivided into types (Types I, II, III, IV, V, VI), with some types further divided into subtypes (e.g., Type II-A, Type II-B, Type II-C, Type V-A, Type V-B).

In certain embodiments, the RGN is a naturally-occurring Type II CRISPR-Cas protein or an active variant or fragment thereof. As used herein, the term “Type II CRISPR-Cas protein,” “Type II CRISPR-Cas effector protein,” or “Type II RNA-guided nuclease” refers to an RGN that requires a trans-activating RNA (tracrRNA) and comprises two nuclease domains (i.e., RuvC and HNH), each of which is responsible for cleaving a single strand of a double-stranded DNA molecule. In some embodiments, the present invention provides a fusion protein comprising a presently disclosed deaminase fused to a Cas9 protein, such as Streptococcus pyogenes Cas9 (SpCas9) or a SpCas9 nickase, the sequences of which are set forth as SEQ ID NOs: 99 and 100, respectively, and are described in U.S. Pat. Nos. 10,000,772 and 8,697,359, each of which is herein incorporated by reference in its entirety. In some embodiments, the present invention provides a fusion protein comprising a presently disclosed deaminase fused to Streptococcus thermophilus Cas9 (StCas9) or a StCas9 nickase, the sequences of which are set forth as SEQ ID NOs: 101 and 102, respectively, and are disclosed in U.S. Pat. No. 10,113,167, which is herein incorporated by reference in its entirety. In some embodiments, the present invention provides a fusion protein comprising a presently disclosed deaminase fused to Streptococcus aureus Cas9 (SaCas9) or a SaCas9 nickase, the sequences of which are set forth as SEQ ID NOs: 103 and 104, respectively, and are disclosed in U.S. Pat. No. 9,752,132, which is herein incorporated by reference in its entirety.

In some embodiments, the CRISPR-Cas protein is a naturally-occurring Type V CRISPR-Cas protein or an active variant or fragment thereof. As used herein, the term “Type V CRISPR-Cas protein,” “Type V CRISPR-Cas effector protein,” or “Type V RNA-guided nuclease” refers to an RGN that cleaves dsDNA and comprises a single RuvC nuclease domain or a split-RuvC nuclease domain and lacks an HNH domain (Zetsche et al 2015, Cell doi:10.1016/j.cell.2015.09.038; Shmakov et al 2017, Nat Rev Microbiol doi:10.1038/nrmicro.2016.184; Yan et al 2018, Science doi:10.1126/science.aav7271; Harrington et al 2018, Science doi: 10.1126/science.aav4294). In some embodiments, a presently disclosed fusion protein comprises a Cas12 (e.g., Cas12a). It is to be noted that Cas12a is also referred to as Cpf1, and does not require a tracrRNA, although other Type V CRISPR-Cas proteins, such as Cas12b, do require a tracrRNA. Most Type V effectors can also target ssDNA (single-stranded DNA), often without a PAM requirement (Zetsche et al 2015; Yan et al 2018; Harrington et al 2018). The terms “Type V CRISPR-Cas protein” and “Type V RGN” encompasses the unique RGNs comprising split RuvC nuclease domains, such as those disclosed in U.S. Provisional Application Nos. 62/955,014 filed Dec. 30, 2019 and 63/058,169 filed Jul. 29, 2020, and PCT International Application No. PCT/US2020/067138 filed Dec. 28, 2020, the contents of each of which are incorporated herein by reference in its entirety. In some embodiments, the present invention provides a fusion protein comprising a presently disclosed deaminase fused to Francisella novicida Cas12a (FnCas12a), the sequence of which is set forth as SEQ ID NOs: 105 and is disclosed in U.S. Pat. No. 9,790,490, which is herein incorporated by reference in its entirety, or any of the nuclease-inactivating mutants of FnCas12a disclosed within U.S. Pat. No. 9,790,490.

In some embodiments, the CRISPR-Cas protein is a naturally-occurring Type VI CRISPR-Cas protein or an active variant or fragment thereof. As used herein, the term “Type VI CRISPR-Cas protein,” “Type VI CRISPR-Cas effector protein,” or “Type VI RGN” refers to a CRISPR-Cas effector protein that does not require a tracrRNA and comprises two HEPN domains that cleave RNA. In some embodiments, the present invention provides a fusion protein comprising a presently disclosed deaminase fused to a Cas13.

In particular embodiments, the presently disclosed fusion proteins comprise an RGN, or a nickase or nuclease-dead variant thereof, listed in Table 1. The guide RNA sequences (crRNA repeat and tracrRNA sequences) that can be used with each RGN of Table 1 are also provided, as well as the consensus PAM sequence. In certain embodiments, the fusion protein comprises an active variant of an RGN (one able to bind to a nucleic acid molecule in an RNA-guided manner) listed in Table 1 having between 80% and 99% or more sequence identity to any one of the amino acid sequences listed in Table 1 including but not limited to about or more than about 80%, about 81%, about 82%, about 83%, about 84%, about 85%, about 86%, about 87%, about 88%, about 89%, about 90%, about 91%, about 92%, about 93%, about 94%, about 95%, 5 about 96%, about 97%, about 98%, about 99%, or more. In particular embodiments, the fusion protein comprises an RGN having 80%, 81%, 82%, 83%, 84%, 85%, 86%, 87%, 88%, 89%, 90%, 91%, 92%, 93%, 94%, 95%, 96%, 97%, 98%, 99%, or more sequence identity to an RGN amino acid sequence disclosed in Table 1. In other embodiments, the fusion protein comprises a fragment of an RGN listed in Table 1 such as one that differs by as few as 1-15 amino acid residues, as few as 1-10, such as 6-10, as few as 5, as few as 4, as few as 3, as few as 2, or as few as 1 amino acid residue. In specific embodiments, the RGN comprises an N-terminal or a C-terminal truncation, which can comprise at least a deletion of 5, 10, 15, 20, 25, 30, 35, 40, 45, 50, 55, 60 amino acids or more from either the N or C terminus of the polypeptide. In some embodiments, the RGN comprises an internal deletion which can comprise at least a deletion of 1, 2, 3, 4, 5, 6, 7, 8, 9, 10, 11, 12, 13, 14, 15, 16, 17, 18, 19, 20, 25, 30, 35, 40, 45, 50, 55, 60 amino acids or more

TABLE 1
Non-limiting examples of RNA-guided nucleases

| | RGN SEQ | crRNA repeat | | |
|---|---|---|---|---|
| RGN Name | ID NO: | sequence | tracrRNA | PAM |
| APG05083.1 | 149 | 150 | 151 | 152 |
| APG07433.1 | 74 | 153 | 154 | 152 |
| APG07513.1 | 155 | 156 | 157 | 152 |
| APG08290.1 | 87 | 158 | 159 | 160 |
| APG05459.1 | 161 | 162 | 163 | 164 |
| APG04583.1 | 165 | 166 | 167 | 168 |
| APG01688.1 | 169 | 170 | 171 | 172 |
| APG00969 | 173 | 174 | 175 | 176 |
| APG03128 | 177 | 178 | 179 | 180 |
| APG09748 | 181 | 182 | 183 | 184 |
| APG00771 | 185 | 186 | 187 | 188 |
| APG02789 | 189 | 190 | 191 | 192 |
| APG09106 | 193 | 194 | 195 | 184 |
| APG05733.1 | 196 | 197 | 198 | 199 |
| APG06207.1 | 200 | 201 | 202 | 203 |
| APG01647.1 | 204 | 205 | 206 | 207 |
| APG08032.1 | 208 | 209 | 210 | 211 |
| APG05712.1 | 212 | 213 | 214 | 215 |
| APG01658.1 | 216 | 217 | 218 | 219 |
| APG06498.1 | 220 | 221 | 222 | 223 |
| APG09106.1 | 224 | 225 | 226 | 227 |
| APG09882.1 | 228 | 229 | 230 | 231 |
| APG02675.1 | 232 | 233 | 234 | 203 |
| APG01405.1 | 235 | 236 | 237 | 238 |
| APG06250.1 | 239 | 240 | 241 | 242 |
| APG06877.1 | 243 | 244 | 245 | 199 |
| APG09053.1 | 246 | 247 | 248 | 249 |
| APG04293.1 | 250 | 251 | 252 | 253 |
| APG01308.1 | 254 | 255 | 256 | 257 |
| APG06646.1 | 258 | 259 | 260 | 253 |
| APG09624 | 261 | 262 | 263 | ND |
| APG05405 | 264 | 265 | 266 | ND |
| APG06622 | 267 | 268 | 269 | 270 |
| APG02787 | 271 | 272 | 273 | 274 |
| APG06248 | 275 | 276 | 277 | 278 |
| APG06007 | 279 | 280 | 281 | 282 |
| APG02874 | 283 | 284 | 285 | 286 |
| APG03850 | 287 | 288 | 289 | 290 |
| APG07553 | 291 | 292 | 293 | 294 |
| APG03031 | 295 | 296 | 297 | 286 |
| APG09208 | 298 | 299 | 300 | 301 |
| APG05586 | 302 | 303 | 304 | 305 |
| APG08770 | 306 | 307 | 308 | 305 |
| APG08167 | 309 | 310 | 311 | 312 |
| APG01604 | 313 | 314 | 315 | 312 |
| APG03021 | 316 | 317 | 318 | 319 |
| APG06015 | 320 | 321 | 322 | 323 |
| APG09344 | 324 | 325 | 326 | 327 |
| APG07991 | 328 | 329 | 330 | 331 |
| APG01868 | 332 | 333 | 334 | 331 |
| APG02998 | 335 | 336 | 337 | 331 |
| APG09298 | 82 | 303 | 304 | 305 |
| APG06251 | 338 | 303 | 304 | 305 |
| APG03066 | 339 | 303 | 304 | 305 |
| APG01560 | 340 | 303 | 304 | 305 |
| APG02777 | 341 | 303 | 304 | 305 |
| APG05761 | 342 | 303 | 304 | 305 |
| APG02479 | 343 | 303 | 304 | 305 |
| APG08385 | 344 | 303 | 304 | 305 |
| APG09217 | 345 | 303 | 304 | 305 |
| APG06657 | 346 | 303 | 304 | 305 |
| APG05586 | 347 | 303 | 304 | 305 |
| APG07433.1 deletion | 106 | 153 | 154 | 152 |
| variant | | | | |
| APG08290.1 deletion | 107 | 158 | 159 | 160 |
| variant | | | | |

* ND: not determined

The term “guide RNA” refers to a nucleotide sequence having sufficient complementarity with a target nucleotide sequence to hybridize with the target sequence and direct sequence-specific binding of an associated RGN to the target nucleotide sequence. For CRISPR-Cas RGNs, the respective guide RNA is one or more RNA molecules (generally, one or two), that can bind to the RGN and guide the RGN to bind to a particular target nucleotide sequence, and in those instances wherein the RGN has nickase or nuclease activity, also cleave the target nucleotide sequence. A guide RNA comprises a CRISPR RNA (crRNA) and in some embodiments, a trans-activating CRISPR RNA (tracrRNA). In some embodiments, a portion of the guideRNA comprises DNA nucleotides. In certain embodiments, the guideRNA comprises artificial, non-naturally-occurring nucleotide analogs or one or more nucleotides are chemically modified.

A CRISPR RNA comprises a spacer sequence and a CRISPR repeat sequence. The “spacer sequence” is the nucleotide sequence that directly hybridizes with the target nucleotide sequence of interest. The spacer sequence is engineered to be fully or partially complementary with the target sequence of interest. In various embodiments, the spacer sequence comprises from about 8 nucleotides to about 30 nucleotides, or more. For example, the spacer sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the spacer sequence is 8, 9, 10, 11, 12, 13, 14, 15, 16, 17, 18, 19, 20, 21, 22, 23, 24, 25, 26, 27, 28, 29, 30, or more nucleotides in length. In some embodiments, the spacer sequence is about 10 to about 26 nucleotides in length, or about 12 to about 30 nucleotides in length. In particular embodiments, the spacer sequence is about 30 nucleotides in length. In some embodiments, the spacer sequence is 30 nucleotides in length. In some embodiments, the degree of complementarity between a spacer sequence and its corresponding target sequence, when optimally aligned using a suitable alignment algorithm, is between 50% and 99% or more, including but not limited to about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the degree of complementarity between a spacer sequence and its corresponding target sequence, when optimally aligned using a suitable alignment algorithm, is 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more. In particular embodiments, the spacer sequence is free of secondary structure, which can be predicted using any suitable polynucleotide folding algorithm known in the art, including but not limited to mFold (see, e.g., Zuker and Stiegler (1981) Nucleic Acids Res. 9:133-148) and RNAfold (see, e.g., Gruber et al. (2008) Cell 106(1):23-24).

The CRISPR RNA repeat sequence comprises a nucleotide sequence that forms a structure, either on its own or in concert with a hybridized tracrRNA, that is recognized by the RGN molecule. In various embodiments, the CRISPR RNA repeat sequence comprises from about 8 nucleotides to about 30 nucleotides, or more. In particular embodiments, the CRISPR RNA repeat sequence comprises from 8 nucleotides to 30 nucleotides, or more. For example, the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In particular embodiments, the CRISPR repeat sequence is 8, 9, 10, 11, 12, 13, 14, 15, 16, 17, 18, 19, 20, 21, 22, 23, 24, 25, 26, 27, 28, 29, 30, or more nucleotides in length. In some embodiments, the degree of complementarity between a CRISPR repeat sequence and its corresponding tracrRNA sequence, when optimally aligned using a suitable alignment algorithm, is between 50% and 99%, or more, including but not limited to about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the degree of complementarity between a CRISPR repeat sequence and its corresponding tracrRNA sequence, when optimally aligned using a suitable alignment algorithm, is 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more.

In some embodiments, the guide RNA further comprises a tracrRNA molecule. A trans-activating CRISPR RNA or tracrRNA molecule comprises a nucleotide sequence comprising a region that has sufficient complementarity to hybridize to a CRISPR repeat sequence of a crRNA, which is referred to herein as the anti-repeat region. In some embodiments, the tracrRNA molecule further comprises a region with secondary structure (e.g., stem-loop) or forms secondary structure upon hybridizing with its corresponding crRNA. In particular embodiments, the region of the tracrRNA that is fully or partially complementary to a CRISPR repeat sequence is at the 5′ end of the molecule and the 3′ end of the tracrRNA comprises secondary structure. This region of secondary structure generally comprises several hairpin structures, including the nexus hairpin, which is found adjacent to the anti-repeat sequence. There are often terminal hairpins at the 3′ end of the tracrRNA that can vary in structure and number, but often comprise a GC-rich Rho-independent transcriptional terminator hairpin followed by a string of Us at the 3′ end. See, for example, Briner et al. (2014) Molecular Cell 56:333-339, Briner and Barrangou (2016) Cold Spring Harb Protoc; doi: 10.1101/pdb.top090902, and U.S. Publication No. 2017/0275648, each of which is herein incorporated by reference in its entirety.

In various embodiments, the anti-repeat region of the tracrRNA that is fully or partially complementary to the CRISPR repeat sequence comprises from about 6 nucleotides to about 30 nucleotides, or more. For example, the region of base pairing between the tracrRNA anti-repeat sequence and the CRISPR repeat sequence can be about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In particular embodiments, the region of base pairing between the tracrRNA anti-repeat sequence and the CRISPR repeat sequence is 6, 7, 8, 9, 10, 11, 12, 13, 14, 15, 16, 17, 18, 19, 20, 21, 22, 23, 24, 25, 26, 27, 28, 29, 30, or more nucleotides in length. In particular embodiments, the anti-repeat region of the tracrRNA that is fully or partially complementary to a CRISPR repeat sequence is about 10 nucleotides in length. In particular embodiments, the anti-repeat region of the tracrRNA that is fully or partially complementary to a CRISPR repeat sequence is 10 nucleotides in length. In some embodiments, the degree of complementarity between a CRISPR repeat sequence and its corresponding tracrRNA anti-repeat sequence, when optimally aligned using a suitable alignment algorithm, is between 50% and 99% or more, including but not limited to about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the degree of complementarity between a CRISPR repeat sequence and its corresponding tracrRNA anti-repeat sequence, when optimally aligned using a suitable alignment algorithm, is 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more.

In various embodiments, the entire tracrRNA comprises from about 60 nucleotides to more than about 210 nucleotides. In particular embodiments, the entire tracrRNA comprises from 60 nucleotides to more than 210 nucleotides. For example, the tracrRNA can be about 60, about 65, about 70, about 75, about 80, about 85, about 90, about 95, about 100, about 105, about 110, about 115, about 120, about 125, about 130, about 135, about 140, about 150, about 160, about 170, about 180, about 190, about 200, about 210 or more nucleotides in length. In particular embodiments, the tracrRNA is 60, 65, 70, 75, 80, 85, 90, 95, 100, 105, 110, 115, 120, 125, 130, 135, 140, 150, 160, 170, 180, 190, 200, 210 or more nucleotides in length. In particular embodiments, the tracrRNA is about 100 to about 200 nucleotides in length, including about 95, about 96, about 97, about 98, about 99, about 100, about 105, about 106, about 107, about 108, about 109, and about 100 nucleotides in length. In particular embodiments, the tracrRNA is 100 to 110 nucleotides in length, including 95, 96, 97, 98, 99, 100, 105, 106, 107, 108, 109, and 110 nucleotides in length.

Guide RNAs form a complex with an RNA-guided, DNA-binding polypeptide or an RNA-guided nuclease to direct the RNA-guided nuclease to bind to a target sequence. If the guide RNA complexes with an RGN, the bound RGN introduces a single-stranded or double-stranded break at the target sequence. After the target sequence has been cleaved, the break can be repaired such that the DNA sequence of the target sequence is modified during the repair process. Provided herein are methods for using mutant variants of RNA-guided nucleases, which are either nuclease inactive or nickases, which are linked to deaminases to modify a target sequence in the DNA of host cells. The mutant variants of RNA-guided nucleases in which the nuclease activity is inactivated or significantly reduced may be referred to as RNA-guided, DNA-binding polypeptides, as the polypeptides are capable of binding to, but not necessarily cleaving, a target sequence. RNA-guided nucleases only capable of cleaving a single strand of a double-stranded nucleic acid molecule are referred to herein as nickases.

A target nucleotide sequence is bound by an RNA-guided, DNA-binding polypeptide and hybridizes with the guide RNA associated with the RGDBP. The target sequence can then be subsequently cleaved if the RGDBP possesses nuclease activity (i.e., is an RGN), which encompasses activity as a nickase.

The guide RNA can be a single guide RNA or a dual-guide RNA system. A single guide RNA comprises the crRNA and optionally tracrRNA on a single molecule of RNA, whereas a dual-guide RNA system comprises a crRNA and a tracrRNA present on two distinct RNA molecules, hybridized to one another through at least a portion of the CRISPR repeat sequence of the crRNA and at least a portion of the tracrRNA, which may be fully or partially complementary to the CRISPR repeat sequence of the crRNA. In some of those embodiments wherein the guide RNA is a single guide RNA, the crRNA and optionally tracrRNA are separated by a linker nucleotide sequence.

In general, the linker nucleotide sequence is one that does not include complementary bases in order to avoid the formation of secondary structure within or comprising nucleotides of the linker nucleotide sequence. In some embodiments, the linker nucleotide sequence between the crRNA and tracrRNA is at least 3, at least 4, at least 5, at least 6, at least 7, at least 8, at least 9, at least 10, at least 11, at least 12, or more nucleotides in length. In particular embodiments, the linker nucleotide sequence between the crRNA and tracrRNA is 3, 4, 5, 6, 7, 8, 9, 10, 11, 12, or more nucleotides in length. In particular embodiments, the linker nucleotide sequence of a single guide RNA is at least 4 nucleotides in length. In particular embodiments, the linker nucleotide sequence of a single guide RNA is 4 nucleotides in length.

In certain embodiments, the guide RNA can be introduced into a target cell, organelle, or embryo as an RNA molecule. The guide RNA can be transcribed in vitro or chemically synthesized. In some embodiments, a nucleotide sequence encoding the guide RNA is introduced into the cell, organelle, or embryo. In some embodiments, the nucleotide sequence encoding the guide RNA is operably linked to a promoter (e.g., an RNA polymerase III promoter). The promoter can be a native promoter or heterologous to the guide RNA-encoding nucleotide sequence. In some embodiments, the promoter is selected from any one of the promoters disclosed in U.S. Provisional Appl. No. 63/209,660, filed Jun. 11, 2021, which is herein incorporated by reference in its entirety, including SEQ ID NOs: 348-357 or an active variant or fragment thereof, including a promoter having at least 60%, at least 70%, at least 80%, at least 85%, at least 90%, at least 95%, at least 96%, at least 97%, at least 98%, at least 99% or greater sequence identity to any one of SEQ ID NOs: 348-357.

In various embodiments, the guide RNA can be introduced into a target cell, organelle, or embryo as a ribonucleoprotein complex, as described herein, wherein the guide RNA is bound to an RNA-guided nuclease polypeptide.

The guide RNA directs an associated RNA-guided nuclease to a particular target nucleotide sequence of interest through hybridization of the guide RNA to the target nucleotide sequence. A target nucleotide sequence can comprise DNA, RNA, or a combination of both and can be single-stranded or double-stranded. A target nucleotide sequence can be genomic DNA (i.e., chromosomal DNA), plasmid DNA, or an RNA molecule (e.g., messenger RNA, ribosomal RNA, transfer RNA, micro RNA, small interfering RNA). The target nucleotide sequence can be bound (and in some embodiments, cleaved) by an RNA-guided, DNA-binding polypeptide in vitro or in a cell. The chromosomal sequence targeted by the RGDBP can be a nuclear, plastid or mitochondrial chromosomal sequence. In some embodiments, the target nucleotide sequence is unique in the target genome.

In some embodiments, the target nucleotide sequence is adjacent to a protospacer adjacent motif (PAM). A PAM is generally within about 1 to about 10 nucleotides from the target nucleotide sequence, including about 1, about 2, about 3, about 4, about 5, about 6, about 7, about 8, about 9, or about 10 nucleotides from the target nucleotide sequence. In particular embodiments, a PAM is within 1 to 10 nucleotides from the target nucleotide sequence, including 1, 2, 3, 4, 5, 6, 7, 8, 9, or 10 nucleotides from the target nucleotide sequence. Unless otherwise stated, the PAM is generally immediately adjacent to the target nucleotide sequence, either at its 5′ or 3′ end. In some embodiments, the PAM is 3′ of the target sequence. Generally, the PAM is a consensus sequence of about 2-6 nucleotides, but in particular embodiments, is 1, 2, 3, 4, 5, 6, 7, 8, 9, or more nucleotides in length.

The PAM restricts which sequences a given RGDBP or RGN can target, as its PAM needs to be proximal to the target nucleotide sequence. Upon recognizing its corresponding PAM sequence, the RGN can cleave the target nucleotide sequence at a specific cleavage site. As used herein, a cleavage site is made up of the two particular nucleotides within a target nucleotide sequence between which the nucleotide sequence is cleaved by an RGN. The cleavage site can comprise the 1st and 2^{nd}, 2^{nd} and 3^{rd}, 3rd and 4^{th}, 4^{th} and 5^{th}, 5^{th} and 6th 7^{th} and 8^{th}, or 8^{th} and 9^{th} nucleotides from the PAM in either the 5′ or 3′ direction. As RGNs can cleave a target nucleotide sequence resulting in staggered ends, in some embodiments, the cleavage site is defined based on the distance of the two nucleotides from the PAM on the positive (+) strand of the polynucleotide and the distance of the two nucleotides from the PAM on the negative (−) strand of the polynucleotide.

RGDBPs and RGNs can be used to deliver a fused polypeptide, polynucleotide, or small molecule payload to a particular genomic location.

In those embodiments wherein the DNA-binding polypeptide comprises a meganuclease, a target sequence can comprise a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. In those embodiments wherein the DNA-binding polypeptide comprises a compact TALEN, the recognition sequence comprises a first CNNNGN sequence that is recognized by the I-TevI domain, followed by a non-specific spacer 4-16 basepairs in length, followed by a second sequence 16-22 bp in length that is recognized by the TAL-effector domain (this sequence typically has a 5′ T base). In those embodiments wherein the DNA-binding polypeptide comprises a zinc finger, the DNA binding domains typically recognize an 18-bp recognition sequence comprising a pair of nine basepair “half-sites” separated by 2-10 basepairs and cleavage by the nuclease creates a blunt end or a 5′ overhang of variable length (frequently four basepairs).

### IV. Fusion Proteins

In some embodiments, a DNA-binding polypeptide (e.g., nuclease-inactive or a nickase RGN) is operably linked to a deaminase of the invention. In some embodiments, a DNA-binding polypeptide (e.g., nuclease inactive RGN or nickase RGN) fused to a deaminase of the invention can be targeted to a particular location of a nucleic acid molecule (i.e., target nucleic acid molecule), which in some embodiments is a particular genomic locus, to alter the expression of a desired sequence. In some embodiments, the binding of a fusion protein to a target sequence results in deamination of a nucleobase, resulting in conversion from one nucleobase to another. In some embodiments, the binding of this fusion protein to a target sequence results in deamination of a nucleobase adjacent to the target sequence. The nucleobase adjacent to the target sequence that is deaminated and mutated using the presently disclosed compositions and methods may be 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 base pairs from the 5′ or 3′ end of the target sequence (bound by the gRNA) within the target nucleic acid molecule. Some aspects of this disclosure provide fusion proteins comprising (i) a DNA-binding polypeptide (e.g., a nuclease-inactive or nickase RGN polypeptide); (ii) a deaminase polypeptide; and optionally (iii) a second deaminase. The second deaminase may be the same deaminase as the first or may be a different deaminase. In some embodiments, both the first and the second deaminase are cytosine deaminases of the invention.

The instant disclosure provides fusion proteins of various configurations. In some embodiments, the deaminase polypeptide is fused to the N-terminus of the DNA-binding polypeptide (e.g., RGN polypeptide). In some embodiments, the deaminase polypeptide is fused to the C-terminus of the DNA-binding polypeptide (e.g., RGN polypeptide).

In some embodiments, the deaminase and DNA-binding polypeptide (e.g., RNA-guided, DNA-binding polypeptide) are fused to each other via a peptide linker. The linker between the deaminase and DNA-binding polypeptide (e.g., RNA-guided, DNA-binding polypeptide) can determine the editing window of the fusion protein, thereby increasing deaminase specificity and reducing off-target mutations. Various linker lengths and flexibilities can be employed, ranging from very flexible linkers of the form (GGGGS)ₙ and (G)ₙ to more rigid linkers of the form (EAAAK)ₙ and (XP)ₙ, to achieve the optimal length and rigidity for deaminase activity for the specific applications. The term “linker,” as used herein, refers to a chemical group or a molecule linking two molecules or moieties, e.g., a binding domain and a cleavage domain of a nuclease. In some embodiments, a linker joins an RNA guided nuclease and a deaminase. In some embodiments, a linker joins a dead or inactive RGN and a deaminase. In further embodiments, a linker joins two deaminases. In some embodiments, a linker joins an RNA guided nuclease and a USP. In some embodiments, a linker joins a deaminase and a USP. In certain embodiments, a linker joins an RNA guided nuclease-deaminase fusion with a USP. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 3-100 amino acids in length, for example, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, a shorter linker is preferred to decrease the overall size or length of the fusion protein or its coding sequence.

In some embodiments, the linker comprises a (GGGGS)ₙ, a (G)ₙ an (EAAAK)ₙ, or an (XP)ₙ motif, or a combination of any of these, wherein n is independently an integer between 1 and 30. In some embodiments, n is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or, if more than one linker or more than one linker motif is present, any combination thereof. Additional suitable linker motifs and linker configurations will be apparent to those of skill in the art. In some embodiments, suitable linker motifs and configurations include those described in Chen et al., 2013 (Adv Drug Deliv Rev. 65(10):1357-69, the entire contents of which are incorporated herein by reference). Additional suitable linker sequences will be apparent to those of skill in the art. In some embodiments, the linker sequence comprises the amino acid sequence set forth as SEQ ID NO: 78 or 79.

In some instances, cellular uracil DNA glycosylase (UDG) recognizes the U:G heteroduplex DNA resulting from the deamination of cytosine and can catalyze the removal of uracil from the DNA to leave an abasic site, thereby initiating base-excision repair with a reversion of the U:G pair to a C:G pair as the most common outcome, although C>G or C>A mutations have also been known to occur, as well as indel (insertion or deletion) formation. In order to prevent or reduce base excision repair by uracil DNA glycosylase that reverts the uracil generated by a cytosine base editor back to a cytosine, in some embodiments, the cytosine base editor fusion protein further comprises a uracil stabilizing polypeptide (USP), such as a uracil DNA glycosylase inhibitor (UGI) or USP2.

As used herein, the terms “uracil stabilizing protein,” “uracil stabilizing polypeptide,” and “USPs” refer to a polypeptide having uracil stabilizing activity. As used herein, the term “uracil stabilizing activity” refers to the ability of a molecule (e.g., a polypeptide) to increase the mutation rate of at least one cytidine, deoxycytidine, or cytosine to a thymidine, deoxythymidine, or thymine in a nucleic acid molecule by a cytosine deaminase compared to the mutation rate by the cytosine deaminase in the absence of the molecule (e.g., uracil stabilizing polypeptide). Without being bound by a theory or mechanism of action, it is believed that USPs may function by maintaining the presence of uracil in single-stranded DNA that has been generated through the deamination of a cytidine, deoxycytidine, or cytosine base for a sufficient period of time to allow for replication to occur and introduce the desired C>T mutation. Uracil stabilizing activity may occur through inhibition of uracil DNA glycosylase, the base excision repair pathway, or mis-match repair mechanisms.

Non-limiting examples of USPs that can be fused to the presently disclosed cytosine deaminases and fusion proteins comprising the presently disclosed cytosine deaminases and a DNA-binding polypeptide include a uracil DNA glycosylase inhibitor (UGI), an example of which is set forth as SEQ ID NO: 86 and any one of the USPs disclosed in PCT Publication No. WO 2021/217002 (which is herein incorporated by reference in its entirety), including USP2 which is set forth herein as SEQ ID NO: 81.

In some embodiments, the deaminase-DBD fusion protein comprises a USP that is a wild-type USP or an active fragment or a variant thereof. For example, in some embodiments, the deaminase-DBD fusion protein comprises a USP comprising SEQ ID NO: 81 or 86 or an active fragment or variant thereof. In some embodiments, a USP fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 81 or 86. In some embodiments, the deaminase-DBD fusion protein comprises a USP having at least 70%, at least 75%, at least 80%, at least 85%, at least 90%, at least 95%, at least 96%, at least 97%, at least 98%, at least 99% or more sequence identity to the USP set forth as SEQ ID NO: 81 or 86.

Additional suitable USP sequences are known to those in the art, and include, for example, those published in Wang et al., 1989. J. Biol. Chem. 264: 1163-1171; Lundquist et al., 1997. J. Biol. Chem. 272:21408-21419; Ravishankar et al., 1998. Nucleic Acids Res. 26:4880-4887; and Putnam et al., 1999. J. Mol. Biol. 287:331-346(1999), the entire contents of each are incorporated herein by reference.

In some embodiments, a linker joins a deaminase-DBD fusion protein with a USP. In some embodiments, a linker joins a deaminase and a USP. In some embodiments, the linker joining a USP to a deaminase or a deaminase-DBD fusion protein is an amino acid or a plurality of amino acids (e.g., a peptide or protein). In some embodiments, the linker is an organic molecule, group, polymer, or chemical moiety. In some embodiments, the linker is 3-100 amino acids in length, for example, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, a shorter linker is preferred to decrease the overall size or length of the fusion protein or its coding sequence. In particular embodiments, the linker joining a USP to a deaminase or a deaminase-DBD fusion protein has the sequence set forth as SEQ ID NO: 120.

In some embodiments, the general architecture of exemplary fusion proteins provided herein comprises the structure: [NH₂]-[deaminase]-[DBP]-[COOH]; [NH₂]-[DBP]-[deaminase]-[COOH]; [NH₂]-[DBP]-[deaminase]-[deaminase]-[COOH]; [NH₂]-[deaminase]-[DBP]-[deaminase]-[COOH]; [NH₂]-[deaminase]-[deaminase]-[DBP]-[COOH]; [NH₂]-[deaminase]-[DBP]-[USP]-[COOH]; [NH₂]-[DBP]-[deaminase]-[USP]-[COOH]; [NH₂]-[USP]-[deaminase]-[DBP]-[COOH]; [NH₂]-[USP]-[DBP]-[deaminase]-[COOH]; [NH₂]-[deaminase]-[USP]-[DBP]-[COOH]; or [NH₂]-[DBP]-[USP]-[deaminase]-[COOH], wherein DBP is a DNA-binding polypeptide, USP is a uracil stabilizing polypeptide, NH₂ is the N-terminus of the fusion protein and COOH is the C-terminus of the fusion protein. In some embodiments, the fusion protein comprises more than two deaminase polypeptides.

In certain embodiments, the general architecture of exemplary fusion proteins provided herein comprises the structure: [NH₂]-[deaminase]-[RGN]-[COOH]; [NH₂]-[RGN]-[deaminase]-[COOH]; [NH₂]-[RGN]-[deaminase]-[deaminase]-[COOH]; [NH₂]-[deaminase]-[RGN]-[deaminase]-[COOH]; or [NH₂]-[deaminase]-[deaminase]-[RGN]-[COOH]; [NH₂]-[deaminase]-[RGN]-[USP]-[COOH]; [NH₂]-[RGN]-[deaminase]-[USP]-[COOH]; [NH₂]-[USP]-[deaminase]-[RGN]-[COOH]; [NH₂]-[USP]-[RGN]-[deaminase]-[COOH]; [NH₂]-[deaminase]-[USP]-[RGN]-[COOH]; or [NH₂]-[RGN]-[USP]-[deaminase]-[COOH], wherein RGN is an RNA-guided nuclease, USP is a uracil stabilizing polypeptide, NH₂ is the N-terminus of the fusion protein and COOH is the C-terminus of the fusion protein. In some embodiments, the fusion protein comprises more than two deaminase polypeptides.

In some embodiments, the fusion protein comprises the structure: [NH₂]-[deaminase]-[nuclease-inactive RGN]-[COOH]; [NH₂]-[deaminase]-[deaminase]-[nuclease-inactive RGN]-[COOH]; [NH₂]-[nuclease-inactive RGN]-[deaminase]-[COOH]; [NH₂]-[deaminase]-[nuclease-inactive RGN]-[deaminase]-[COOH]; [NH₂]-[nuclease-inactive RGN]-[deaminase]-[deaminase]-[COOH]; [NH₂]-[deaminase]-[nuclease-inactive RGN]-[USP]-[COOH]; [NH₂]-[nuclease-inactive RGN]-[deaminase]-[USP]-[COOH]; [NH₂]-[USP]-[deaminase]-[nuclease-inactive RGN]-[COOH]; [NH₂]-[USP]-[nuclease-inactive RGN]-[deaminase]-[COOH]; [NH₂]-[deaminase]-[USP]-[nuclease-inactive RGN]-[COOH]; or [NH₂]-[nuclease-inactive RGN]-[USP]-[deaminase]-[COOH]. It should be understood that “nuclease-inactive RGN” represents any RGN, including any CRISPR-Cas protein, which has been mutated to be nuclease-inactive. In some embodiments, the fusion protein comprises more than two deaminase polypeptides.

In some embodiments, the fusion protein comprises the structure: [NH₂]-[deaminase]-[RGN nickase]-[COOH]; [NH₂]-[deaminase]-[deaminase]-[RGN nickase]-[COOH]; [NH₂]-[RGN nickase]-[deaminase]-[COOH]; [NH₂]-[deaminase]-[RGN nickase]-[deaminase]-[COOH]; or [NH₂]-[RGN nickase]-[deaminase]-[deaminase]-[COOH]; [NH₂]-[deaminase]-[RGN nickase]-[USP]-[COOH]; [NH₂]-[RGN nickase]-[deaminase]-[USP]-[COOH]; [NH₂]-[USP]-[deaminase]-[RGN nickase]-[COOH]; [NH₂]-[USP]-[RGN nickase]-[deaminase]-[COOH]; [NH₂]-[deaminase]-[USP]-[RGN nickase]-[COOH]; or [NH₂]-[RGN nickase]-[USP]-[deaminase]-[COOH]. It should be understood that “RGN nickase” represents any RGN, including any CRISPR-Cas protein, which has been mutated to be active as a nickase.

In some embodiments, the “-” used in the general architecture above indicates the presence of an optional linker sequence. In some embodiments, the fusion proteins provided herein do not comprise a linker sequence. In some embodiments, at least one of the optional linker sequences are present.

Other exemplary features that may be present are localization sequences, such as nuclear localization sequences, cytoplasmic localization sequences, export sequences, such as nuclear export sequences, or other localization sequences, as well as sequence tags that are useful for solubilization, purification or detection of the fusion proteins. Suitable localization signal sequences and sequences of protein tags that are provided herein, and include, but are not limited to, biotin carboxylase carrier protein (BCCP) tags, myc-tags, calmodulin-tags, FLAG-tags (e.g., 3×FLAG-tag), hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), streptags, biotin ligase tags, FlAsH tags, V5 tags, and SBP-tags.

Additional suitable sequences will be apparent to those of skill in the art.

In certain embodiments, the presently disclosed fusion proteins comprise at least one cell-penetrating domain that facilitates cellular uptake of the fusion protein. Cell-penetrating domains are known in the art and generally comprise stretches of positively charged amino acid residues (i.e., polycationic cell-penetrating domains), alternating polar amino acid residues and non-polar amino acid residues (i.e., amphipathic cell-penetrating domains), or hydrophobic amino acid residues (i.e., hydrophobic cell-penetrating domains) (see, e.g., Milletti F. (2012) Drug Discov Today 17:850-860). A non-limiting example of a cell-penetrating domain is the trans-activating transcriptional activator (TAT) from the human immunodeficiency virus 1.

In some embodiments, deaminases or fusion proteins provided herein further comprise a nuclear localization sequence (NLS). The nuclear localization signal, plastid localization signal, mitochondrial localization signal, dual-targeting localization signal, and/or cell-penetrating domain can be located at the amino-terminus (N-terminus), the carboxyl-terminus (C-terminus), or in an internal location of the fusion protein.

In some embodiments, the NLS is fused to the N-terminus of the fusion protein or deaminase. In some embodiments, the NLS is fused to the C-terminus of the fusion protein or deaminase. In some embodiments, the NLS is fused to the N-terminus of the deaminase of the fusion protein. In some embodiments, the NLS is fused to the C-terminus of the deaminase of the fusion protein. In some embodiments, the NLS is fused to the N-terminus of the DNA-binding polypeptide (e.g., RGN polypeptide) of the fusion protein. In some embodiments, the NLS is fused to the C-terminus of the DNA-binding polypeptide (e.g., RGN polypeptide) of the fusion protein. In some embodiments, the NLS is fused to the N-terminus of the deaminase polypeptide of the fusion protein. In some embodiments, the NLS is fused to the C-terminus of the deaminase polypeptide of the fusion protein. In some embodiments, the NLS is fused to the fusion protein via one or more linkers, including but not limited to SEQ ID NO: 148. In some embodiments, the NLS is fused to the fusion protein without a linker. In some embodiments, the NLS comprises an amino acid sequence of any one of the NLS sequences provided or referenced herein. In some embodiments, the NLS comprises an amino acid sequence as set forth in SEQ ID NO: 76 or SEQ ID NO: 80. In some embodiments, the fusion protein or deaminase comprises SEQ ID NO: 76 on its N-terminus and SEQ ID NO: 80 on its C-terminus.

In some embodiments, fusion proteins as provided herein comprise the full-length sequence of a deaminase, e.g., any one of SEQ ID NO: 2, 4, and 6-12. In some embodiments, however, fusion proteins as provided herein do not comprise a full-length sequence of a deaminase, but only a fragment thereof. For example, in some embodiments, a fusion protein provided herein further comprises a DNA-binding polypeptide (e.g., an RNA-guided, DNA-binding) domain and a deaminase domain.

In some embodiments, a fusion protein of the invention comprises a DNA-binding polypeptide (e.g., an RGN) and a deaminase, wherein the deaminase has an amino acid sequence having at least 50%, at least 55%, at least 60%, at least 65%, at least 70%, at least 75%, at least 80%, at least 85%, at least 90%, at least 95%, at least 96%, at least 97%, at least 98%, at least 99%, or 100% identity to any of SEQ ID NOs: 2, 4, and 6-12. Examples of such fusion proteins are described in the Examples section herein.

In some embodiments, the fusion protein comprises one deaminase polypeptide. In some embodiments, the fusion protein comprises at least two deaminase polypeptides, operably linked either directly or via a peptide linker. In some embodiments, the fusion protein comprises one deaminase polypeptide, and a second deaminase polypeptide is co-expressed with the fusion protein.

Also provided herein is a ribonucleoprotein complex comprising a fusion protein comprising a deaminase and an RGDBP and the guide RNA, either as a single guide or as a dual guide RNA (also collectively referred to as gRNA).

V. Nucleotides Encoding Deaminases, Fusion Proteins, and or gRNA

The present disclosure provides polynucleotides (SEQ ID NOs: 109, 111, and 113-119) encoding the presently disclosed deaminase polypeptides. The present disclosure further provides polynucleotides encoding for fusion proteins which comprise a deaminase and DNA-binding polypeptide, for example a meganuclease, a zinc finger fusion protein, or a TALEN. The present disclosure further provides polynucleotides encoding for fusion proteins which comprise a deaminase and an RNA-guided, DNA-binding polypeptide. Such RNA-guided, DNA-binding polypeptides may be an RGN or RGN variant. The protein variant may be nuclease-inactive or a nickase. The RGN may be a CRISPR-Cas protein or active variant or fragment thereof. SEQ ID NOs: 74 and 75 are non-limiting examples of an RGN and a nickase RGN variant, respectively. Examples of CRISPR-Cas nucleases are well-known in the art, and similar corresponding mutations can create mutant variants which are also nickases or are nuclease inactive.

An embodiment of the invention provides a polynucleotide encoding a fusion protein which comprises an RGDBP and a deaminase described herein (SEQ ID NO: 2, 4, and 6-12, or a variant thereof). In some embodiments, a second polynucleotide encodes the guide RNA required by the RGDBP for targeting to the nucleotide sequence of interest. In some embodiments, the guide RNA and the fusion protein are encoded by the same polynucleotide.

The use of the term “polynucleotide” is not intended to limit the present disclosure to polynucleotides comprising DNA, though such DNA polynucleotides are contemplated. Those of ordinary skill in the art will recognize that polynucleotides can comprise ribonucleotides (RNA) (e.g., mRNA) and combinations of ribonucleotides and deoxyribonucleotides. Such deoxyribonucleotides and ribonucleotides include both naturally occurring molecules and synthetic analogues. The polynucleotides disclosed herein also encompass all forms of sequences including, but not limited to, single-stranded forms, double-stranded forms, stem-and-loop structures, circular forms (e.g., including circular RNA), and the like.

An embodiment of the invention is a nucleic acid molecule comprising a sequence having at least 50%, at least 55%, at least 60%, at least 65%, at least 70%, at least 75%, at least 80%, at least 85%, at least 90%, at least 95%, at least 96%, at least 97%, at least 98%, at least 99% or 100% identity to any of SEQ ID NOs: 109, 111, and 113-119, wherein the nucleic acid molecule encodes a deaminase having cytosine deaminase activity. The nucleic acid molecule may further comprise a heterologous promoter or terminator. The nucleic acid molecule may encode a fusion protein, where the encoded deaminase is operably linked to a DNA-binding polypeptide, optionally a second deaminase, and optionally a USP. In some embodiments, the nucleic acid molecule encodes a fusion protein, where the encoded deaminase is operably linked to an RGN, optionally a second deaminase, and optionally a USP.

In some embodiments, nucleic acid molecules comprising a polynucleotide which encodes a deaminase of the invention are codon optimized for expression in an organism of interest. A “codon-optimized” coding sequence is a polynucleotide coding sequence having its frequency of codon usage designed to mimic the frequency of preferred codon usage or transcription conditions of a particular host cell. Expression in the particular host cell or organism is enhanced as a result of the alteration of one or more codons at the nucleic acid level such that the translated amino acid sequence is not changed. Nucleic acid molecules can be codon optimized, either wholly or in part. Codon tables and other references providing preference information for a wide range of organisms are available in the art (see, e.g., Campbell and Gowri (1990) Plant Physiol. 92:1-11 for a discussion of plant-preferred codon usage). Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

In some embodiments, polynucleotides encoding the deaminases, fusion proteins, and/or gRNAs described herein are provided in expression cassettes for in vitro expression or expression in a cell, organelle, embryo, or organism of interest. The cassette may include 5′ and 3′ regulatory sequences operably linked to a polynucleotide encoding a deaminase and/or a fusion protein comprising a deaminase, an RNA-guided DNA-binding polypeptide and optionally a second deaminase, and/or gRNA provided herein that allows for expression of the polynucleotide. The cassette may additionally contain at least one additional gene or genetic element to be cotransformed into the organism. Where additional genes or elements are included, the components are operably linked. The term “operably linked” is intended to mean a functional linkage between two or more elements. For example, an operable linkage between a promoter and a coding region of interest (e.g., a region coding for a deaminase, RNA-guided DNA-binding polypeptide, and/or gRNA) is a functional link that allows for expression of the coding region of interest. Operably linked elements may be contiguous or non-contiguous. When used to refer to the joining of two protein coding regions, by operably linked is intended that the coding regions are in the same reading frame. In some embodiments, the additional gene(s) or element(s) are provided on multiple expression cassettes. For example, the nucleotide sequence encoding a presently disclosed deaminase, either alone or as a component of a fusion protein, can be present on one expression cassette, whereas the nucleotide sequence encoding a gRNA can be on a separate expression cassette. Another example may have the nucleotide sequence encoding a presently disclosed deaminase alone on a first expression cassette, a second expression cassette encoding a fusion protein comprising a deaminase, and a nucleotide sequence encoding a gRNA on a third expression cassette. Such an expression cassette is provided with a plurality of restriction sites and/or recombination sites for insertion of the polynucleotides to be under the transcriptional regulation of the regulatory regions. Expression cassettes which comprise a selectable marker gene may also be present.

The expression cassette may include in the 5-3′ direction of transcription, a transcriptional (and, in some embodiments, translational) initiation region (i.e., a promoter), a deaminase-encoding polynucleotide of the invention, and a transcriptional (and in some embodiments, translational) termination region (i.e., termination region) functional in the organism of interest. The promoters of the invention are capable of directing or driving expression of a coding sequence in a host cell. The regulatory regions (e.g., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention. As used herein, a chimeric gene comprises a coding sequence operably linked to a transcription initiation region that is heterologous to the coding sequence.

Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Additional regulatory signals include, but are not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, inducible, growth stage-specific, cell type-specific, tissue-preferred, tissue-specific, or other promoters for expression in the organism of interest. See, for example, promoters set forth in WO 99/43838 and in U.S. Pat. Nos. 8,575,425; 7,790,846; 8,147,856; 8,58,6832; 7,772,369; 7,534,939; 6,072,050; 5,659,026; 5,608,149; 5,608,144; 5,604,121; 5,569,597; 5,466,785; 5,399,680; 5,268,463; 5,608,142; and 6,177,611; herein incorporated by reference.

For expression in plants, constitutive promoters also include CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); and MAS (Velten et al. (1984) EMBO J. 3:2723-2730).

Examples of inducible promoters are the Adh1 promoter which is inducible by hypoxia or cold stress, the Hsp70 promoter which is inducible by heat stress, the PPDK promoter and the pepcarboxylase promoter which are both inducible by light. Also useful are promoters which are chemically inducible, such as the In2-2 promoter which is safener induced (U.S. Pat. No. 5,364,780), the AxigI promoter which is auxin induced and tapetum specific but also active in callus (PCT US01/22169), the steroid-responsive promoters (see, for example, the ERE promoter which is estrogen induced, and the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

In some embodiments, tissue-specific or tissue-preferred promoters are utilized to target expression of an expression construct within a particular tissue. In certain embodiments, the tissue-specific or tissue-preferred promoters are active in plant tissue. Examples of promoters under developmental control in plants include promoters that initiate transcription preferentially in certain tissues, such as leaves, roots, fruit, seeds, or flowers. A “tissue specific” promoter is a promoter that initiates transcription only in certain tissues. Unlike constitutive expression of genes, tissue-specific expression is the result of several interacting levels of gene regulation. As such, promoters from homologous or closely related plant species can be preferable to use to achieve efficient and reliable expression of transgenes in particular tissues. In some embodiments, the expression comprises a tissue-preferred promoter. A “tissue preferred” promoter is a promoter that initiates transcription preferentially, but not necessarily entirely or solely in certain tissues.

In some embodiments, the nucleic acid molecules encoding a deaminase described herein comprise a cell type-specific promoter. A “cell type specific” promoter is a promoter that primarily drives expression in certain cell types in one or more organs. Some examples of plant cells in which cell type specific promoters functional in plants may be primarily active include, for example, BETL cells, vascular cells in roots, leaves, stalk cells, and stem cells. The nucleic acid molecules can also include cell type preferred promoters. A “cell type preferred” promoter is a promoter that primarily drives expression mostly, but not necessarily entirely or solely in certain cell types in one or more organs. Some examples of plant cells in which cell type preferred promoters functional in plants may be preferentially active include, for example, BETL cells, vascular cells in roots, leaves, stalk cells, and stem cells.

In some embodiments, the nucleic acid sequences encoding the deaminases, fusion proteins, and/or gRNAs are operably linked to a promoter sequence that is recognized by a phage RNA polymerase for example, for in vitro mRNA synthesis. In such embodiments, the in vitro-transcribed RNA can be purified for use in the methods described herein. For example, the promoter sequence can be a T7, T3, or SP6 promoter sequence or a variation of a T7, T3, or SP6 promoter sequence. In such embodiments, the expressed protein and/or RNAs can be purified for use in the methods of genome modification described herein.

In certain embodiments, the polynucleotide encoding the deaminase, fusion protein, and/or gRNA is linked to a polyadenylation signal (e.g., SV40 polyA signal and other signals functional in plants) and/or at least one transcriptional termination sequence. In some embodiments, the sequence encoding the deaminase or fusion protein is linked to sequence(s) encoding at least one nuclear localization signal, at least one cell-penetrating domain, and/or at least one signal peptide capable of trafficking proteins to particular subcellular locations, as described elsewhere herein.

In some embodiments, the polynucleotide encoding the deaminase, fusion protein, and/or gRNA is present in a vector or multiple vectors. A “vector” refers to a polynucleotide composition for transferring, delivering, or introducing a nucleic acid into a host cell. Suitable vectors include plasmid vectors, phagemids, cosmids, artificial/mini-chromosomes, transposons, and viral vectors (e.g., lentiviral vectors, adeno-associated viral vectors, baculoviral vector). In some embodiments, the vector comprises additional expression control sequences (e.g., enhancer sequences, Kozak sequences, polyadenylation sequences, transcriptional termination sequences), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001.

In some embodiments, the vector comprises a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D).

In some embodiments, the expression cassette or vector comprising the sequence encoding a fusion protein comprising an RNA-guided DNA-binding polypeptide, such as an RGN, further comprises a sequence encoding a gRNA. In some embodiments, the sequence(s) encoding the gRNA are operably linked to at least one transcriptional control sequence for expression of the gRNA in the organism or host cell of interest. For example, the polynucleotide encoding the gRNA can be operably linked to a promoter sequence that is recognized by RNA polymerase III (Pol III). Examples of suitable Pol III promoters include, but are not limited to, mammalian U6, U3, H1, and 7SL RNA promoters, rice U6 and U3 promoters.

As indicated, expression constructs comprising nucleotide sequences encoding the deaminases, fusion proteins, and/or gRNAs can be used to transform organisms of interest. Methods for transformation involve introducing a nucleotide construct into an organism of interest. By “introducing” is intended to introduce the nucleotide construct to the host cell in such a manner that the construct gains access to the interior of the host cell. The methods of the invention do not require a particular method for introducing a nucleotide construct to a host organism, only that the nucleotide construct gains access to the interior of at least one cell of the host organism. In some embodiments, an mRNA encoding a deaminase or a fusion protein is introduced into a host cell. In some embodiments wherein the fusion protein comprises a RGDBP, an mRNA encoding the fusion protein is introduced into a cell and a gRNA is introduced into the cell. The host cell can be a eukaryotic or prokaryotic cell. In particular embodiments, the eukaryotic host cell is a plant cell, a mammalian cell, or an insect cell. Methods for introducing nucleotide constructs into plants and other host cells are known in the art including, but not limited to, stable transformation methods, transient transformation methods, and virus-mediated methods.

The methods result in a transformed organism, such as a plant, including whole plants, as well as plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells, pollen).

“Transgenic organisms” or “transformed organisms” or “stably transformed” organisms or cells or tissues refers to organisms that have incorporated or integrated a polynucleotide encoding a deaminase of the invention. It is recognized that other exogenous or endogenous nucleic acid sequences or DNA fragments may also be incorporated into the host cell. Agrobacterium-and biolistic-mediated transformation remain the two predominantly employed approaches for transformation of plant cells. However, transformation of a host cell may be performed by infection, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, and viral mediated, liposome mediated and the like. Viral-mediated introduction of a polynucleotide encoding a deaminase, fusion protein, and/or gRNA includes retroviral, lentiviral, adenoviral, and adeno-associated viral mediated introduction and expression, as well as the use of Caulimoviruses (e.g., cauliflower mosaic virus), Geminiviruses (e.g., bean golden yellow mosaic virus or maize streak virus), and RNA plant viruses (e.g., tobacco mosaic virus).

Transformation protocols as well as protocols for introducing polypeptides or polynucleotide sequences into plants may vary depending on the type of host cell (e.g., monocot or dicot plant cell) targeted for transformation. Methods for transformation are known in the art and include those set forth in U.S. Pat. Nos. 8,575,425; 7,692,068; 8,802,934; 7,541,517; each of which is herein incorporated by reference. See, also, Rakoczy-Trojanowska, M. (2002) Cell Mol Biol Lett. 7:849-858; Jones et al. (2005) Plant Methods 1:5; Rivera et al. (2012) Physics of Life Reviews 9:308-345; Bartlett et al. (2008) Plant Methods 4:1-12; Bates, G. W. (1999) Methods in Molecular Biology 111:359-366; Binns and Thomashow (1988) Annual Reviews in Microbiology 42:575-606; Christou, P. (1992) The Plant Journal 2:275-281; Christou, P. (1995) Euphytica 85:13-27; Tzfira et al. (2004) TRENDS in Genetics 20:375-383; Yao et al. (2006) Journal of Experimental Botany 57:3737-3746; Zupan and Zambryski (1995) Plant Physiology 107:1041-1047; Jones et al. (2005) Plant Methods 1:5;

Transformation may result in stable or transient incorporation of the nucleic acid into the cell. “Stable transformation” is intended to mean that the nucleotide construct introduced into a host cell integrates into the genome of the host cell and is capable of being inherited by the progeny thereof. “Transient transformation” is intended to mean that a polynucleotide is introduced into the host cell and does not integrate into the genome of the host cell.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

The cells that have been transformed may be grown into a transgenic organism, such as a plant, in accordance with conventional ways. See, for example, McCormick et al. (1986) Plant Cell Reports 5:81-84. These plants may then be grown, and either pollinated with the same transformed strain or different strains, and the resulting hybrid having the deaminase or fusion protein polynucleotide identified. Two or more generations may be grown to ensure that the deaminase or fusion protein polynucleotide is stably maintained and inherited and then seeds harvested to ensure the presence of the deaminase or fusion protein polynucleotide. In this manner, the present invention provides transformed seed (also referred to as “transgenic seed”) having a nucleotide construct of the invention, for example, an expression cassette of the invention, stably incorporated into their genome.

In some embodiments, cells that have been transformed are introduced into an organism. These cells could have originated from the organism, wherein the cells are transformed in an ex vivo approach.

The sequences provided herein may be used for transformation of any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers. Vegetables include, but are not limited to, tomatoes, lettuce, green beans, lima beans, peas, and members of the genus Cucumis such as cucumber, cantaloupe, and musk melon. Ornamentals include, but are not limited to, azalea, hydrangea, hibiscus, roses, tulips, daffodils, petunias, carnation, poinsettia, and chrysanthemum. Preferably, plants of the present invention are crop plants (for example, maize, sorghum, wheat, sunflower, tomato, crucifers, peppers, potato, cotton, rice, soybean, sugarbeet, sugarcane, tobacco, barley, oilseed rape, etc.).

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides. Further provided is a processed plant product or byproduct that retains the sequences disclosed herein, including for example, soymeal.

In some embodiments, the polynucleotides encoding the deaminases, fusion proteins, and/or gRNAs are used to transform any eukaryotic species, including but not limited to animals (e.g., mammals, insects, fish, birds, and reptiles), fungi, amoeba, algae, and yeast. In some embodiments, the polynucleotides encoding the deaminases, fusion proteins, and/or gRNAs are used to transform any prokaryotic species, including but not limited to, archaea and bacteria (e.g., Bacillus spp., Klebsiella spp. Streptomyces spp., Rhizobium spp., Escherichia spp., Pseudomonas spp., Salmonella spp., Shigella spp., Vibrio spp., Yersinia spp., Mycoplasma spp., Agrobacterium spp., and Lactobacillus spp.).

In some embodiments, conventional viral and non-viral based gene transfer methods are used to introduce nucleic acids in mammalian cells or target tissues. Such methods can be used to administer nucleic acids encoding a deaminase or fusion protein of the invention and optionally a gRNA to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. Non-limiting examples include vectors utilizing Caulimoviruses (e.g., cauliflower mosaic virus), Geminiviruses (e.g., bean golden yellow mosaic virus or maize steak virus), and RNA plant viruses (e.g., tobacco mosaic virus). For a review of gene therapy procedures, see Anderson, Science 256: 808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology, Doerfler and Bohm (eds) (1995); and Yu et al., Gene Therapy 1:13-26 (1994).

Methods of non-viral delivery of nucleic acids include lipofection, Agrobacterium-mediated transformation, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration). The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids takes advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Katin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Packaging cells are typically used to form virus particles that are capable of infecting a host cell. Such cells include 293 cells, which package adenovirus, and ψJ² cells or PA317 cells, which package retrovirus.

Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences.

The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

Ideally, the coding sequence of an RGN-deaminase fusion protein of the invention and a corresponding guide RNA for targeting the fusion protein may all be packaged into a single AAV vector. The generally accepted size limit for AAV vectors is 4.7 kb, although larger sizes may be contemplated at the expense of reduced packing efficiency. To ensure that the expression cassettes for both the fusion protein and its corresponding guide RNA could fit into an AAV vector, novel, active deletion variants of RGNs such as those set forth as SEQ ID NOs: 97, 98, 106, and 107 or active deletion variants of deaminases may be used such as those described herein set forth as SEQ ID NOs: 2, 4, and 6. In addition to shortening the amino acid sequence and therefore the coding sequence of the RGN and/or the deaminase of the fusion protein, the peptide linker which links the RGN and the deaminase may also be shortened. The USP, if present, and the linker connecting the USP and the RGN-deaminase fusion protein may be shortened. Finally, the genetic elements, such as the promoters, enhancers, and/or terminators, may also be engineered via deletion analysis to determine the minimal size required for each to be functional. The present invention also teaches methods of using said fusion proteins for targeted base editing through in vivo AAV vector delivery.

In some embodiments, a host cell is transiently or non-transiently transfected with one or more vectors described herein. In some embodiments, a cell is transfected as it naturally occurs in a subject. In some embodiments, a cell that is transfected is taken from a subject.

In some embodiments, a cell that is transfected is a eukaryotic cell. In some embodiments, the eukaryotic cell is an animal cell (e.g., mammals, insects, fish, birds, and reptiles). In some embodiments, a cell that is transfected is a human cell. In some embodiments, a cell that is transfected is a cell of hematopoietic origin, such as an immune cell (i.e., a cell of the innate or adaptive immune system) including but not limited to a B cell, a T cell, a natural killer (NK) cell, a pluripotent stem cell, an induced pluripotent stem cell, a chimeric antigen receptor T (CAR-T) cell, a monocyte, a macrophage, and a dendritic cell.

In some embodiments, the cell is derived from cells taken from a subject, such as a cell line. In some embodiments, the cell or cell line is prokaryotic. In some embodiments, the cell or cell line is eukaryotic. In further embodiments, the cell or cell line is derived from insect, avian, plant, or fungal species. In some embodiments, the cell or cell line may be mammalian, such as for example human, monkey, mouse, cow, swine, goat, hamster, rat, cat, or dog. A wide variety of cell lines for tissue culture are known in the art. Examples of cell lines include, but are not limited to, C8161, CCRF-CEM, MOLT, mIMCD-3, NHDF, HeLaS3, Huhl, Huh4, Huh7, HUVEC, HASMC, HEKn, HEKa, MiaPaCell, Panel, PC-3, TFl, CTLL-2, CIR, Rat6, CVI, RPTE, AlO, T24, 182, A375, ARH-77, Calul, SW480, SW620, SKOV3, SK-UT, CaCo2, P388D1, SEM-K2, WEHI-231, HB56, TIB55, lurkat, 145.01, LRMB, Bcl-1, BC-3, IC21, DLD2, Raw264.7, NRK, NRK-52E, MRC5, MEF, Hep G2, HeLa B, HeLa T4. COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-I cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L235010, CORL23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1clc7, HL-60, HMEC, HT-29, lurkat, lY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCKII, MDCKII, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

In some embodiments, a cell transfected with one or more vectors described herein is used to establish a new cell line comprising one or more vector-derived sequences. In some embodiments, a cell transiently transfected with a fusion protein of the invention and optionally a gRNA, or with a ribonucleoprotein complex of the invention, and modified through the activity of a fusion protein or ribonucleoprotein complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some embodiments, one or more vectors described herein are used to produce a non-human transgenic animal or transgenic plant. In some embodiments, the transgenic animal is an insect. In further embodiments, the insect is an insect pest, such as a mosquito or tick. In some embodiments, the insect is a plant pest, such as a corn rootworm or a fall armyworm. In some embodiments, the transgenic animal is a bird, such as a chicken, turkey, goose, or duck. In some embodiments, the transgenic animal is a mammal, such as a human, mouse, rat, hamster, monkey, ape, rabbit, swine, cow, horse, goat, sheep, cat, or dog.

### VI. Variants and Fragments of Polypeptides and Polynucleotides

The present disclosure provides cytosine deaminases which are active on DNA molecules, the amino acid sequence of which are set forth as SEQ ID NO: 2, 4, and 6-12, active variants or fragments thereof, and polynucleotides encoding the same.

While the activity of a variant or fragment may be altered compared to the polynucleotide or polypeptide of interest, the variant and fragment should retain the functionality of the polynucleotide or polypeptide of interest. For example, a variant or fragment may have increased activity, decreased activity, different spectrum of activity or any other alteration in activity when compared to the polynucleotide or polypeptide of interest.

Fragments and variants of deaminases of the invention which have cytosine deaminase activity will retain said activity if they are part of a fusion protein further comprising a DNA-binding polypeptide or a fragment thereof.

The term “fragment” refers to a portion of a polynucleotide or polypeptide sequence of the invention. “Fragments” or “biologically active portions” include polynucleotides comprising a sufficient number of contiguous nucleotides to retain the biological activity (i.e., deaminase activity on nucleic acids). “Fragments” or “biologically active portions” include polypeptides comprising a sufficient number of contiguous amino acid residues to retain the biological activity. Fragments of the deaminases disclosed herein include those that are shorter than the full-length sequences due to the use of an alternate downstream start site. In some embodiments, a biologically active portion of a deaminase is a polypeptide that comprises, for example, 10, 20, 30, 40, 50, 60, 70, 80, 90, 100, 110, 120, 130, 140, 150, 160, or more contiguous amino acid residues of any of SEQ ID NOs: 2, 4, and 6-12, or a variant thereof. Such biologically active portions can be prepared by recombinant techniques and evaluated for activity.

In general, “variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” or “wild type” polynucleotide or polypeptide comprises a naturally occurring nucleotide sequence or amino acid sequence, respectively. For polynucleotides, conservative variants include those sequences that, because of the degeneracy of the genetic code, encode the native amino acid sequence of the gene of interest. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode the polypeptide or the polynucleotide of interest. Generally, variants of a particular polynucleotide disclosed herein will have at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more sequence identity to that particular polynucleotide as determined by sequence alignment programs and parameters described elsewhere herein.

Variants of a particular polynucleotide disclosed herein (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides disclosed herein is evaluated by comparison of the percent sequence identity shared by the two polypeptides they encode, the percent sequence identity between the two encoded polypeptides is at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more sequence identity.

In particular embodiments, the presently disclosed polynucleotides encode a cytosine deaminase comprising an amino acid sequence having at least 40%, at least 45%, at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or greater identity to an amino acid sequence of any of SEQ ID NOs: 2, 4, and 6-12.

A biologically active variant of a cytosine deaminase of the invention may differ by as few as 1-15 amino acid residues, as few as 1-10, such as 6-10, as few as 5, as few as 4, as few as 3, as few as 2, or as few as 1 amino acid residue. In specific embodiments, the polypeptides comprise an N-terminal or a C-terminal truncation, which can comprise at least a deletion of 5, 10, 15, 20, 25, 30, 35, 40, 45, 50, 55, 60 amino acids or more from either the N or C terminus of the polypeptide. In some embodiments, the polypeptides comprise an internal deletion which can comprise at least a deletion of 1, 2, 3, 4, 5, 6, 7, 8, 9, 10, 11, 12, 13, 14, 15, 16, 17, 18, 19, 20, 25, 30, 35, 40, 45, 50, 55, 60 amino acids or more.

In some embodiments, a biologically active polypeptide variant of SEQ ID NO: 2 does not comprise amino acid residues 1-12 or 195-230 of SEQ ID NO: 1. In certain embodiments, a biologically active variant of SEQ ID NO: 4 does not comprise amino acid residues 1-12 or 198-201 of SEQ ID NO: 3. In particular embodiments, a biologically active variant of SEQ ID NO: 6 does not comprise amino acid residues 1-15 of SEQ ID NO: 5. In certain embodiments, the deaminase has an amino acid sequence having at least 90% sequence identity to SEQ ID NO: 2 and does not comprise amino acid residues 1-12 or 195-230 of SEQ ID NO: 1. In some embodiments, the deaminase has an amino acid sequence having at least 95% sequence identity to SEQ ID NO: 4 and does not comprise amino acid residues 1-12 or 198-201 of SEQ ID NO: 3. In some embodiments, the deaminase has an amino acid sequence having at least 95% sequence identity to SEQ ID NO: 6 and does not comprise amino acid residues 1-15 of SEQ ID NO: 5.

It is recognized that modifications may be made to the deaminases provided herein creating variant proteins and polynucleotides. Changes designed by man may be introduced through the application of site-directed mutagenesis techniques. In some embodiments, native, as yet-unknown or as yet unidentified polynucleotides and/or polypeptides structurally and/or functionally-related to the sequences disclosed herein may also be identified that fall within the scope of the present invention. Conservative amino acid substitutions may be made in nonconserved regions that do not alter the function of the polypeptide as a cytosine deaminase. In some embodiments, modifications are made that improve the cytosine deaminase activity of the deaminase.

Variant polynucleotides and proteins also encompass sequences and proteins derived from a mutagenic and recombinogenic procedure such as DNA shuffling. With such a procedure, one or more different deaminases disclosed herein (e.g., SEQ ID NOs: 2, 4, and 6-12) is manipulated to create a new cytosine deaminase possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the deaminase sequences provided herein and other subsequently identified deaminase genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458. A “shuffled” nucleic acid is a nucleic acid produced by a shuffling procedure such as any shuffling procedure set forth herein. Shuffled nucleic acids are produced by recombining (physically or virtually) two or more nucleic acids (or character strings), for example in an artificial, and optionally recursive, fashion. Generally, one or more screening steps are used in shuffling processes to identify nucleic acids of interest; this screening step can be performed before or after any recombination step. In some (but not all) shuffling embodiments, it is desirable to perform multiple rounds of recombination prior to selection to increase the diversity of the pool to be screened. The overall process of recombination and selection are optionally repeated recursively. Depending on context, shuffling can refer to an overall process of recombination and selection, or, alternately, can simply refer to the recombinational portions of the overall process.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well-known in the art and described, e.g., in Dayhoff et al. (1978) “A model of evolutionary change in proteins.” In “Atlas of Protein Sequence and Structure,” Vol. 5, Suppl. 3 (ed. M. O. Dayhoff), pp. 345-352. Natl. Biomed. Res. Found., Washington, D.C. and Henikoff et al. (1992) Proc. Natl. Acad. Sci. USA 89:10915-10919. The BLOSUM62 matrix is often used as a default scoring substitution matrix in sequence alignment protocols. The gap existence penalty is imposed for the introduction of a single amino acid gap in one of the aligned sequences, and the gap extension penalty is imposed for each additional empty amino acid position inserted into an already opened gap. The alignment is defined by the amino acids positions of each sequence at which the alignment begins and ends, and optionally by the insertion of a gap or multiple gaps in one or both sequences, so as to arrive at the highest possible score. While optimal alignment and scoring can be accomplished manually, the process is facilitated by the use of a computer-implemented alignment algorithm, e.g., gapped BLAST 2.0, described in Altschul et al. (1997) Nucleic Acids Res. 25:3389-3402, and made available to the public at the National Center for Biotechnology Information Website (www.ncbi.nlm.nih.gov). Optimal alignments, including multiple alignments, can be prepared using, e.g., PSI-BLAST, available through www.ncbi.nlm.nih.gov and described by Altschul et al. (1997) Nucleic Acids Res. 25:3389-3402.

With respect to an amino acid sequence that is optimally aligned with a reference sequence, an amino acid residue “corresponds to” the position in the reference sequence with which the residue is paired in the alignment. The “position” is denoted by a number that sequentially identifies each amino acid in the reference sequence based on its position relative to the N-terminus. Owing to deletions, insertion, truncations, fusions, etc., that must be taken into account when determining an optimal alignment, in general the amino acid residue number in a test sequence as determined by simply counting from the N-terminal will not necessarily be the same as the number of its corresponding position in the reference sequence. For example, in a case where there is a deletion in an aligned test sequence, there will be no amino acid that corresponds to a position in the reference sequence at the site of deletion. Where there is an insertion in an aligned reference sequence, that insertion will not correspond to any amino acid position in the reference sequence. In the case of truncations or fusions there can be stretches of amino acids in either the reference or aligned sequence that do not correspond to any amino acid in the corresponding sequence.

### VII. Antibodies

Antibodies to the deaminases, fusion proteins, or ribonucleoproteins comprising the deaminases of the present invention, including those having the amino acid sequence set forth as any one of SEQ ID NOs: 2, 4, and 6-12 or active variants or fragments thereof, are also encompassed. Methods for producing antibodies are well known in the art (see, for example, Harlow and Lane (1988) Antibodies: A Laboratory Manual, Cold Spring Harbor Laboratory, Cold Spring Harbor, N.Y.; and U.S. Pat. No. 4,196,265). These antibodies can be used in kits for the detection and isolation of deaminases or fusion proteins or ribonucleoproteins comprising deaminases described herein. Thus, this disclosure provides kits comprising antibodies that specifically bind to the polypeptides or ribonucleoproteins described herein, including, for example, polypeptides comprising a sequence of at least 85% identity to any of SEQ ID NOs: 2, 4, and 6-12.

### VIII. Systems and Ribonucleoprotein Complexes for Binding and or Modifying a Target Sequence of Interest and Methods of Making the Same

The present disclosure provides a system which targets to a nucleic acid sequence and modifies a target nucleic acid sequence. In some embodiments, an RNA-guided, DNA-binding polypeptide, such as an RGN, and the gRNA are responsible for targeting the ribonucleoprotein complex to a nucleic acid sequence of interest; the deaminase polypeptide fused to the RGDBP is responsible for modifying the targeted nucleic acid sequence from C>N. In some embodiments, the deaminase converts C>T. In some embodiments, the deaminase converts C>G. The guide RNA hybridizes to the target sequence of interest and also forms a complex with the RNA-guided, DNA-binding polypeptide, thereby directing the RNA-guided, DNA-binding polypeptide to bind to the target sequence. The RNA-guided, DNA-binding polypeptide is part of a fusion protein that also comprises a deaminase described herein. In some embodiments, the RNA-guided, DNA-binding polypeptide is an RGN, such as a Cas9. Other examples of RNA-guided, DNA-binding polypeptides include RGNs such as those described in International Patent Application Publication Nos. WO 2019/236566 and WO 2020/139783, each of which is incorporated by reference in its entirety. In some embodiments, the RNA-guided, DNA-binding polypeptide is a Type II CRISPR-Cas polypeptide, or an active variant or fragment thereof. In some embodiments, the RNA-guided, DNA-binding polypeptide is a Type V CRISPR-Cas polypeptide, or an active variant or fragment thereof. In some embodiments, the RNA-guided, DNA-binding polypeptide is a Type VI CRISPR-Cas polypeptide. In some embodiments, the DNA-binding polypeptide of the fusion protein does not require an RNA guide, such as a zinc finger nuclease, TALEN, or meganuclease polypeptide. In some embodiments, the nuclease activity of a DNA-binding polypeptide has been partially or completely inactivated. In further embodiments, the RNA-guided, DNA-binding polypeptide comprises an amino acid sequence of an RGN, such as for example APG07433.1 (SEQ ID NO: 74), or an active variant or fragment thereof such as nickase nAPG07433.1 (SEQ ID NO: 75) or other nickase RGN variants (SEQ ID NOs: 75 and 88-98).

In some embodiments, the system for binding and modifying a target sequence of interest provided herein is a ribonucleoprotein complex, which is at least one molecule of an RNA bound to at least one protein. The ribonucleoprotein complexes provided herein comprise at least one guide RNA as the RNA component and a fusion protein comprising a deaminase of the invention and an RNA-guided, DNA-binding polypeptide as the protein component. In some embodiments, the ribonucleoprotein complex is purified from a cell or organism that has been transformed with polynucleotides that encode the fusion protein and a guide RNA and cultured under conditions to allow for the expression of the fusion protein and guide RNA.

Methods are provided for making a deaminase, a fusion protein, or a fusion protein ribonucleoprotein complex. Such methods comprise culturing a cell comprising a nucleotide sequence encoding a deaminase, a fusion protein, and in some embodiments a nucleotide sequence encoding a guide RNA, under conditions in which the deaminase or fusion protein (and in some embodiments, the guide RNA) is expressed. The deaminase, fusion protein, or fusion ribonucleoprotein can then be purified from a lysate of the cultured cells.

Methods for purifying a deaminase, fusion protein, or fusion ribonucleoprotein complex from a lysate of a biological sample are known in the art (e.g., size exclusion and/or affinity chromatography, 2D-PAGE, HPLC, reversed-phase chromatography, immunoprecipitation). In particular methods, the deaminase or fusion protein is recombinantly produced and comprises a purification tag to aid in its purification, including but not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, biotin carboxyl carrier protein (BCCP), and calmodulin. Generally, the tagged deaminase, fusion protein, or fusion ribonucleoprotein complex is purified using immunoprecipitation or other similar methods known in the art.

An “isolated” or “purified” polypeptide, or biologically active portion thereof, is substantially or essentially free from components that normally accompany or interact with the polypeptide as found in its naturally occurring environment. Thus, an isolated or purified polypeptide is substantially free of other cellular material, or culture medium when produced by recombinant techniques, or substantially free of chemical precursors or other chemicals when chemically synthesized. A protein that is substantially free of cellular material includes preparations of protein having less than 30%, less than 20%, less than 10%, less than 5%, or less than 1% (by dry weight) of contaminating protein. When the protein of the invention or biologically active portion thereof is recombinantly produced, optimally culture medium represents less than 30%, less than 20%, less than 10%, less than 5%, or less than 1% (by dry weight) of chemical precursors or non-protein-of-interest chemicals.

Particular methods provided herein for binding and/or cleaving a target sequence of interest involve the use of an in vitro assembled ribonucleoprotein complex. In vitro assembly of a ribonucleoprotein complex can be performed using any method known in the art in which an RGDBP polypeptide or a fusion protein comprising the same is contacted with a guide RNA under conditions to allow for binding of the RGDBP polypeptide or fusion protein comprising the same to the guide RNA. As used herein, “contact”, “contacting”, “contacted,” refer to placing the components of a desired reaction together under conditions suitable for carrying out the desired reaction. The RGDBP polypeptide or fusion protein comprising the same can be purified from a biological sample, cell lysate, or culture medium, produced via in vitro translation, or chemically synthesized. The guide RNA can be purified from a biological sample, cell lysate, or culture medium, transcribed in vitro, or chemically synthesized. The RGDBP polypeptide or fusion protein comprising the same and guide RNA can be brought into contact in solution (e.g., buffered saline solution) to allow for in vitro assembly of the ribonucleoprotein complex.

### IX. Methods of Modifying a Target Sequence

The present disclosure provides methods for modifying a target nucleic acid molecule (e.g., target DNA molecule) of interest. The methods include delivering a fusion protein comprising a DNA-binding polypeptide and at least one deaminase of the invention or a polynucleotide encoding the same to a target sequence or a cell, organelle, or embryo comprising a target sequence. In certain embodiments, the methods include delivering a system comprising at least one guide RNA or a polynucleotide encoding the same, and at least one fusion protein comprising at least one deaminase of the invention and an RNA-guided, DNA-binding polypeptide or a polynucleotide encoding the same to the target sequence or a cell, organelle, or embryo comprising the target sequence. In some embodiments, the fusion protein comprises any one of the amino acid sequences of SEQ ID NOs: 2, 4, and 6-12, or an active variant or fragment thereof.

In some embodiments, the methods comprise contacting a DNA molecule with (a) a fusion protein comprising a deaminase and an RNA-guided, DNA-binding polypeptide, such as for example a nuclease-inactive or a nickase RGN; and (b) a gRNA targeting the fusion protein of (a) to a target nucleotide sequence of the DNA molecule; wherein the DNA molecule is contacted with the fusion protein and the gRNA in an amount effective and under conditions suitable for the deamination of a nucleobase. In some embodiments, the target DNA molecule comprises a sequence associated with a disease or disorder, and wherein the deamination of the nucleobase results in a sequence that is not associated with a disease or disorder. In some embodiments, the disease or disorder affects animals. In further embodiments, the disease or disorder affects mammals, such as humans, cows, horses, dogs, cats, goats, sheep, swine, monkeys, rats, mice, or hamsters. In some embodiments, the target DNA sequence resides in an allele of a crop plant, wherein the particular allele of the trait of interest results in a plant of lesser agronomic value. The deamination of the nucleobase results in an allele that improves the trait and increases the agronomic value of the plant.

In those embodiments wherein the method comprises delivering a polynucleotide encoding a guide RNA and/or a fusion protein, the cell or embryo can then be cultured under conditions in which the guide RNA and/or fusion protein are expressed. In various embodiments, the method comprises contacting a target sequence with a ribonucleoprotein complex comprising a gRNA and a fusion protein (which comprises a deaminase of the invention and an RNA-guided DNA-binding polypeptide). In certain embodiments, the method comprises introducing into a cell, organelle, or embryo comprising a target sequence a ribonucleoprotein complex of the invention. The ribonucleoprotein complex of the invention can be one that has been purified from a biological sample, recombinantly produced and subsequently purified, or in vitro-assembled as described herein. In those embodiments wherein the ribonucleoprotein complex that is contacted with the target sequence or a cell organelle, or embryo has been assembled in vitro, the method can further comprise the in vitro assembly of the complex prior to contact with the target sequence, cell, organelle, or embryo.

A purified or in vitro assembled ribonucleoprotein complex of the invention can be introduced into a cell, organelle, or embryo using any method known in the art, including, but not limited to electroporation. In some embodiments, a fusion protein comprising a deaminase of the invention and an RNA-guided, DNA-binding polypeptide, and a polynucleotide encoding or comprising the guide RNA is introduced into a cell, organelle, or embryo using any method known in the art (e.g., electroporation).

Upon delivery to or contact with the target sequence or cell, organelle, or embryo comprising the target sequence, the guide RNA directs the fusion protein to bind to the target sequence in a sequence-specific manner. The target sequence can subsequently be modified via the deaminase of the fusion protein. In some embodiments, the binding of this fusion protein to a target sequence results in modification of a nucleotide adjacent to the target sequence. The nucleobase adjacent to the target sequence that is modified by the deaminase may be 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 base pairs from the 5′ or 3′ end of the target sequence. A fusion protein comprising a deaminase of the invention and an RNA-guided, DNA-binding polypeptide can introduce targeted C>N mutations in the targeted DNA molecule. In some embodiments, the fusion protein introduces targeted C>T mutations in the targeted DNA molecule. In certain embodiments, the fusion protein introduces targeted C>G mutations in the targeted DNA molecule.

Methods to measure binding of the fusion protein to a target sequence are known in the art and include chromatin immunoprecipitation assays, gel mobility shift assays, DNA pull-down assays, reporter assays, microplate capture and detection assays. Likewise, methods to measure cleavage or modification of a target sequence are known in the art and include in vitro or in vivo cleavage assays wherein cleavage is confirmed using PCR, sequencing, or gel electrophoresis, with or without the attachment of an appropriate label (e.g., radioisotope, fluorescent substance) to the target sequence to facilitate detection of degradation products. In some embodiments, the nicking triggered exponential amplification reaction (NTEXPAR) assay is used (see, e.g., Zhang et al. (2016) Chem. Sci. 7:4951-4957). In vivo cleavage can be evaluated using the Surveyor assay (Guschin et al. (2010) Methods Mol Biol 649:247-256).

In some embodiments, the methods involve the use of an RNA-binding, DNA-guided polypeptide, as part of the fusion protein, complexed with more than one guide RNA. The more than one guide RNA can target different regions of a single gene or can target multiple genes. This multiple targeting enables the deaminase of the fusion protein to modify nucleic acids, thereby introducing multiple mutations in the target nucleic acid molecule (e.g., genome) of interest.

In those embodiments wherein the method involves the use of an RNA-guided nuclease (RGN), such as a nickase RGN (i.e., is only able to cleave a single strand of a double-stranded polynucleotide, for example nAPG07433.1 (SEQ ID NO: 75 or SEQ ID NOs: 88-98), the method can comprise introducing two different RGNs or RGN variants that target identical or overlapping target sequences and cleave different strands of the polynucleotide. For example, an RGN nickase that only cleaves the positive (+) strand of a double-stranded polynucleotide can be introduced along with a second RGN nickase that only cleaves the negative (−) strand of a double-stranded polynucleotide. In some embodiments, two different fusion proteins are provided, where each fusion protein comprises a different RGN with a different PAM recognition sequence, so that a greater diversity of nucleotide sequences may be targeted for mutation.

One of ordinary skill in the art will appreciate that any of the presently disclosed methods can be used to target a single target sequence or multiple target sequences. Thus, methods comprise the use of a fusion protein comprising a single RNA-guided, DNA-binding polypeptide in combination with multiple, distinct guide RNAs, which can target multiple, distinct sequences within a single gene and/or multiple genes. The deaminase of the fusion protein would then introduce mutations at each of the targeted sequences. Also encompassed herein are methods wherein multiple, distinct guide RNAs are introduced in combination with multiple, distinct RNA-guided, DNA binding polypeptides. Such RNA-guided, DNA-binding polypeptides may be multiple RGN or RGN variants. These guide RNAs and guide RNA/fusion protein systems can target multiple, distinct sequences within a single gene and/or multiple genes.

In some embodiments, a fusion protein comprising an RNA-guided, DNA-binding polypeptide and a deaminase polypeptide of the invention may be used for generating mutations in a targeted gene or targeted region of a gene of interest. In some embodiments, a fusion protein of the invention may be used for saturation mutagenesis of a targeted gene or region of a targeted gene of interest followed by high-throughput forward genetic screening to identify novel mutations and/or phenotypes. In some embodiments, a fusion protein described herein may be used for generating mutations in a targeted genomic location, which may or may not comprise coding DNA sequence. Libraries of cell lines generated by the targeted mutagenesis described above may also be useful for study of gene function or gene expression.

### X. Target Polynucleotides

In one aspect, the invention provides for methods of modifying a target polynucleotide in a eukaryotic cell, which may be in vivo, ex vivo or in vitro. In some embodiments, the method comprises sampling a cell or population of cells from a human or non-human animal or plant (including microalgae) and modifying the cell or cells. Culturing may occur at any stage ex vivo. The cell or cells may even be re-introduced into the human, non-human animal or plant (including micro-algae).

Using natural variability, plant breeders combine most useful genes for desirable qualities, such as yield, quality, uniformity, hardiness, and resistance against pests. These desirable qualities also include growth, day length preferences, temperature requirements, initiation date of floral or reproductive development, fatty acid content, insect resistance, disease resistance, nematode resistance, fungal resistance, herbicide resistance, tolerance to various environmental factors including drought, heat, wet, cold, wind, and adverse soil conditions including high salinity. The sources of these useful genes include native or foreign varieties, heirloom varieties, wild plant relatives, and induced mutations, e.g., treating plant material with mutagenic agents. Using the present invention, plant breeders are provided with a new tool to induce mutations. Accordingly, one skilled in the art can employ the present invention to induce the rise of useful genes, with more precision than previous mutagenic agents and hence accelerate and improve plant breeding programs.

The target polynucleotide of a deaminase or a fusion protein of the invention can be any polynucleotide endogenous or exogenous to the eukaryotic cell. For example, the target polynucleotide can be a polynucleotide residing in the nucleus of the eukaryotic cell. In some embodiments, the target polynucleotide is a sequence coding a gene product (e.g., a protein) or a non-coding sequence (e.g., a regulatory polynucleotide or a junk DNA). In some embodiments, the target sequence for a fusion protein of the invention is associated with a PAM (protospacer adjacent motif); that is, a short sequence recognized by the RNA-guided DNA-binding polypeptide. The precise sequence and length requirements for the PAM differ depending on the RNA-guided DNA-binding polypeptide used, but PAMs are typically 2-5 base pair sequences adjacent the protospacer (that is, the target sequence).

The target polynucleotide of a fusion protein of the invention may include a number of disease-associated genes and polynucleotides as well as signaling biochemical pathway-associated genes and polynucleotides. Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissue compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease (e.g., a causal mutation). The transcribed or translated products may be known or unknown, and further may be at a normal or abnormal level.

Non-limiting examples of disease-associated genes that can be targeted using the presently disclosed methods and compositions are provided in Table 23. In some embodiments, the disease-associated gene that is targeted are those disclosed in Table 23 having a T>C or G>C mutation. Additional examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.), available on the World Wide Web.

In some embodiments, the methods comprise contacting a DNA molecule comprising a target DNA sequence with a DNA-binding polypeptide-deaminase fusion protein of the invention, wherein the DNA molecule is contacted with the fusion protein in an amount effective and under conditions suitable for the deamination of a nucleobase. In certain embodiments, the methods comprise contacting a DNA molecule comprising a target DNA sequence with (a) an RGN-deaminase fusion protein of the invention; and (b) a gRNA targeting the fusion protein of (a) to a target nucleotide sequence of the DNA strand; wherein the DNA molecule is contacted with the fusion protein and the gRNA in an amount effective and under conditions suitable for the deamination of a nucleobase. In some embodiments, the target DNA sequence comprises a sequence associated with a disease or disorder, and wherein the deamination of the nucleobase results in a sequence that is not associated with a disease or disorder. In some embodiments, the target DNA sequence resides in an allele of a crop plant, wherein the particular allele of the trait of interest results in a plant of lesser agronomic value. The deamination of the nucleobase results in an allele that improves the trait and increases the agronomic value of the plant.

In some embodiments, the target DNA sequence comprises a T>C or G>C point mutation associated with a disease or disorder, and wherein the deamination of the mutant C base results in a sequence that is not associated with a disease or disorder. In some embodiments, the deamination corrects a point mutation in the sequence associated with the disease or disorder.

In some embodiments, the sequence associated with the disease or disorder encodes a protein, and the deamination introduces a stop codon into the sequence associated with the disease or disorder, resulting in a truncation of the encoded protein. In some embodiments, the contacting is performed in vivo in a subject susceptible to having, having, or diagnosed with the disease or disorder. In some embodiments, the disease or disorder is a disease associated with a point mutation, or a single-base mutation, in the genome. In some embodiments, the disease is a genetic disease, a cancer, a metabolic disease, or a lysosomal storage disease.

### XI. Pharmaceutical Compositions and Methods of Treatment

Methods of treating a disease in a subject in need thereof are provided herein. The methods comprise administering to a subject in need thereof an effective amount of a presently disclosed fusion protein or a polynucleotide encoding the same, a presently disclosed gRNA or a polynucleotide encoding the same, a presently disclosed fusion protein system, or a cell modified by or comprising any one of these compositions.

In some embodiments, the treatment comprises in vivo gene editing by administering to a subject in need thereof a presently disclosed fusion protein, gRNA, or a presently disclosed fusion protein system or polynucleotide(s) encoding the same. In some embodiments, the treatment comprises ex vivo gene editing wherein cells are genetically modified ex vivo with a presently disclosed fusion protein, gRNA, or a presently disclosed fusion protein system or polynucleotide(s) encoding the same and then the modified cells are administered to a subject. In some embodiments, the genetically modified cells originate from the subject that is then administered the modified cells, and the transplanted cells are referred to herein as autologous. In some embodiments, the genetically modified cells originate from a different subject (i.e., donor) within the same species as the subject that is administered the modified cells (i.e., recipient), and the transplanted cells are referred to herein as allogeneic. In some examples described herein, the cells can be expanded in culture prior to administration to a subject in need thereof.

In some embodiments, the disease to be treated with the presently disclosed compositions is one that can be treated with immunotherapy, such as with a chimeric antigen receptor (CAR) T cell. Such diseases include but are not limited to cancer.

In some embodiments, the deamination of the target nucleobase results in the correction of a genetic defect or in the correction of a point mutation that leads to a loss of function in a gene product. In some embodiments, the genetic defect is associated with a disease or disorder, e.g., a lysosomal storage disorder or a metabolic disease, such as, for example, type I diabetes. Thus, in some embodiments, the disease to be treated with the presently disclosed compositions is associated with a sequence (i.e., the sequence is causal for the disease or disorder or causal for symptoms associated with the disease or disorder) that is mutated in order to treat the disease or disorder or the reduction of symptoms associated with the disease or disorder.

In some embodiments, the disease to be treated with the presently disclosed compositions is associated with a causal mutation. As used herein, a “causal mutation” refers to a particular nucleotide, nucleotides, or nucleotide sequence in the genome that contributes to the severity or presence of a disease or disorder in a subject. The correction of the causal mutation leads to the improvement of at least one symptom resulting from a disease or disorder. In some embodiments, the correction of the causal mutation leads to the improvement of at least one symptom resulting from a disease or disorder. In some embodiments, the causal mutation is adjacent to a PAM site recognized by the RGDBP (e.g., RGN) fused to a deaminase disclosed herein. The causal mutation can be corrected with a fusion polypeptide comprising a RGDBP (e.g., RGN) and a presently disclosed deaminase. Non-limiting examples of diseases associated with a causal mutation include cystic fibrosis, Niemann-Pick disease, diseases caused by splice site disruptions, and the diseases listed in Table 23. Additional non-limiting examples of disease-associated genes and mutations are available from McKusick-Nathans Institute of Genetic Medicine, Johns Hopkins University (Baltimore, Md.) and National Center for Biotechnology Information, National Library of Medicine (Bethesda, Md.), available on the World Wide Web.

In some embodiments, the methods provided herein are used to introduce a deactivating point mutation into a gene or allele that encodes a gene product that is associated with a disease or disorder. For example, in some embodiments, methods are provided herein that employ a fusion protein to introduce a deactivating point mutation into an oncogene (e.g., in the treatment of a proliferative disease). A deactivating mutation may, in some embodiments, generate a premature stop codon in a coding sequence, which results in the expression of a truncated gene product, e.g., a truncated protein lacking the function of the full-length protein. In some embodiments, the purpose of the methods provided herein is to restore the function of a dysfunctional gene via genome editing. The fusion proteins provided herein can be validated for gene editing-based human therapeutics in vitro, e.g., by correcting a disease associated mutation in human cell culture. It will be understood by the skilled artisan that the fusion proteins provided herein, e.g., the fusion proteins comprising an RNA-guided, DNA-binding polypeptide and deaminase polypeptide can be used to correct any single point T>C or G>C mutation. Deamination of the mutant C to T or G leads to a correction of the mutation.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, and the delivery system in which it is carried.

The term “administering” refers to the placement of an active ingredient into a subject, by a method or route that results in at least partial localization of the introduced active ingredient at a desired site, such as a site of injury or repair, such that a desired effect(s) is produced. In those embodiments wherein cells are administered, the cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the patient, i.e., long-term engraftment. For example, in some aspects described herein, an effective amount of photoreceptor cells or retinal progenitor cells is administered via a systemic route of administration, such as an intraperitoneal or intravenous route.

In some embodiments, the administering comprises administering by viral delivery. In some embodiments, the administering comprises administering by electroporation. In some embodiments, the administering comprises administering by nanoparticle delivery. In some embodiments, the administering comprises administering by liposome delivery. Any effective route of administration can be used to administer an effective amount of a pharmaceutical composition described herein. In some embodiments, the administering comprises administering by a method selected from the group consisting of: intravenously, subcutaneously, intramuscularly, orally, rectally, by aerosol, parenterally, ophthalmicly, pulmonarily, transdermally, vaginally, otically, nasally, and by topical administration, or any combination thereof. In some embodiments, for the delivery of cells, administration by injection or infusion is used.

As used herein, the term “subject” refers to any individual for whom diagnosis, treatment or therapy is desired. In some embodiments, the subject is an animal. In some embodiments, the subject is a mammal. In some embodiments, the subject is a human being.

The efficacy of a treatment can be determined by the skilled clinician. However, a treatment is considered an “effective treatment,” if any one or all of the signs or symptoms of a disease or disorder are altered in a beneficial manner (e.g., decreased by at least 10%), or other clinically accepted symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art. Treatment includes: (1) inhibiting the disease, e.g., arresting, or slowing the progression of symptoms; or (2) relieving the disease, e.g., causing regression of symptoms; and (3) preventing or reducing the likelihood of the development of symptoms.

Pharmaceutical compositions comprising the presently disclosed RGN polypeptides or polynucleotides encoding the same, the presently disclosed gRNAs or polynucleotides encoding the same, the presently disclosed deaminases or polynucleotides encoding the same, the presently disclosed fusion proteins, the presently disclosed systems (such as those comprising a fusion protein), or cells comprising any of the RGN polypeptides or RGN-encoding polynucleotides, gRNA or gRNA-encoding polynucleotides, fusion protein-encoding polynucleotides, or the systems, and a pharmaceutically acceptable carrier are provided.

As used herein, a “pharmaceutically acceptable carrier” refers to a material that does not cause significant irritation to an organism and does not abrogate the activity and properties of the active ingredient (e.g., a deaminase or fusion protein or nucleic acid molecule encoding the same). Carriers must be of sufficiently high purity and of sufficiently low toxicity to render them suitable for administration to a subject being treated. The carrier can be inert, or it can possess pharmaceutical benefits. In some embodiments, a pharmaceutically acceptable carrier comprises one or more compatible solid or liquid filler, diluents or encapsulating substances which are suitable for administration to a human or other vertebrate animal. In some embodiments, the pharmaceutical composition comprises a pharmaceutically acceptable carrier that is non-naturally occurring. In some embodiments, the pharmaceutically acceptable carrier and the active ingredient are not found together in nature and are thus, heterologous.

Pharmaceutical compositions used in the presently disclosed methods can be formulated with suitable carriers, excipients, and other agents that provide suitable transfer, delivery, tolerance, and the like. A multitude of appropriate formulations are known to those skilled in the art. See, e.g., Remington, The Science and Practice of Pharmacy (21st ed. 2005). Non-limiting examples include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. Administered intravenously, particular carriers are physiological saline or phosphate buffered saline (PBS). Pharmaceutical compositions for oral or parenteral use may be prepared into dosage forms in a unit dose suited to fit a dose of the active ingredients. Such dosage forms in a unit dose include, for example, tablets, pills, capsules, injections (ampoules), suppositories, etc. These compositions also may contain adjuvants including preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

In some embodiments wherein cells comprising or modified with the presently disclosed RGNs, gRNAs, deaminases, fusion proteins, systems (including those comprising fusion proteins) or polynucleotides encoding the same are administered to a subject, the cells are administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the cells described herein using routine experimentation.

A cell composition can also be emulsified or presented as a liposome composition, provided that the emulsification procedure does not adversely affect cell viability. The cells and any other active ingredient can be mixed with excipients that are pharmaceutically acceptable and compatible with the active ingredient, and in amounts suitable for use in the therapeutic methods described herein.

Additional agents included in a cell composition can include pharmaceutically acceptable salts of the components therein. Pharmaceutically acceptable salts include the acid addition salts (formed with the free amino groups of the polypeptide) that are formed with inorganic acids, such as, for example, hydrochloric or phosphoric acids, or such organic acids as acetic, tartaric, mandelic and the like. Salts formed with the free carboxyl groups can also be derived from inorganic bases, such as, for example, sodium, potassium, ammonium, calcium or ferric hydroxides, and such organic bases as isopropylamine, trimethylamine, 2-ethylamino ethanol, histidine, procaine and the like.

### Modifying Causal Mutations Using Base-Editing

An example of a genetically inherited disease which could be corrected using an approach that relies on an RGN-deaminase fusion protein of the invention is Niemann-Pick disease Type C. Niemann-Pick disease (NPC) is an autosomal recessive lysosomal storage disorder caused by mutations in the NPC1 or NPC2 gene (the sequence of the NPC1 gene is set forth as SEQ ID NO: 121), which results in abnormal accumulation of cholesterol and glycosphingolipids (GSLs). Patients with NPC typically develop symptoms between four and seven years of age. Major symptoms include liver and lung disease, hypotonia, dysphagia, delayed psychomotor development, cerebellar ataxia, progressive cognitive impairment, dementia, and other neurological dysfunctions. A common variant associated with juvenile neurologic disease onset is NM_000271.5(NPC1):c.3182T>C (p.Ile1061Thr) in exon 21, which is correctable with cytosine base editing. The present invention also discloses potential target sequences which guide the fusion proteins of the invention to target the causal mutations of various diseases, including the NM_000271.5(NPC1):c.3182T>C (p.I1061T) mutation in exon 21 known to cause Niemann-Pick disease type C.

### XII. Cells Comprising a Polynucleotide Genetic Modification

Provided herein are cells and organisms comprising a target nucleic acid molecule of interest that has been modified using a process mediated by a fusion protein, optionally with a gRNA, as described herein. In some embodiments, the fusion protein comprises a deaminase polypeptide comprising an amino acid sequence of any of SEQ ID NOs: 2, 4, and 6-12, or an active variant or fragment thereof. In some embodiments, the fusion protein comprises a cytosine deaminase comprising an amino acid sequence having at least 50%, at least 55%, at least 60%, at least 65%, at least 70%, at least 75%, at least 80%, at least 85%, at least 90%, at least 95%, at least 96%, at least 97%, at least 98%, or at least 99% identity to any of SEQ ID NOs: 2, 4, and 6-12. In some embodiments, the fusion protein comprises a deaminase and a DNA-binding polypeptide (e.g., an RNA-guided, DNA-binding polypeptide). In further embodiments, the fusion protein comprises a deaminase and an RGN or a variant thereof, such as for example APG07433.1 (SEQ ID NO: 74) or its nickase variant nAPG07433.1 (SEQ ID NO: 75). In some embodiments, the fusion protein comprises a deaminase and a Cas9 or a variant thereof, such as for example dCas9 or nickase Cas9. In some embodiments, the fusion protein comprises a nuclease-inactive or nickase variant of a Type II CRISPR-Cas polypeptide. In some embodiments, the fusion protein comprises a nuclease-inactive or nickase variant of a Type V CRISPR-Cas polypeptide. In some embodiments, the fusion protein comprises a nuclease-inactive or nickase variant of a Type VI CRISPR-Cas polypeptide.

The modified cells can be eukaryotic (e.g., mammalian, plant, insect, avian cell) or prokaryotic. Also provided are organelles and embryos comprising at least one nucleotide sequence that has been modified by a process utilizing a fusion protein as described herein. The genetically modified cells, organisms, organelles, and embryos can be heterozygous or homozygous for the modified nucleotide sequence. The mutation(s) introduced by the deaminase of the fusion protein can result in altered expression (up-regulation or down-regulation), inactivation, or the expression of an altered protein product or an integrated sequence. In those instances wherein the mutation(s) results in either the inactivation of a gene or the expression of a non-functional protein product, the genetically modified cell, organism, organelle, or embryo is referred to as a “knock out”. The knock out phenotype can be the result of a deletion mutation (i.e., deletion of at least one nucleotide), an insertion mutation (i.e., insertion of at least one nucleotide), or a nonsense mutation (i.e., substitution of at least one nucleotide such that a stop codon is introduced).

In some embodiments, the mutation(s) introduced by the deaminase of the fusion protein results in the production of a variant protein product. The expressed variant protein product can have at least one amino acid substitution and/or the addition or deletion of at least one amino acid. The variant protein product can exhibit modified characteristics or activities when compared to the wild-type protein, including but not limited to altered enzymatic activity or substrate specificity.

In some embodiments, the mutation(s) introduced by the deaminase of the fusion protein result in an altered expression pattern of a protein. As a non-limiting example, mutation(s) in the regulatory regions controlling the expression of a protein product can result in the overexpression or downregulation of the protein product or an altered tissue or temporal expression pattern.

The cells that have been modified can be grown into an organism, such as a plant, in accordance with conventional ways. See, for example, McCormick et al. (1986) Plant Cell Reports 5:81-84. These plants may then be grown, and either pollinated with the same modified strain or different strains, and the resulting hybrid having the genetic modification. The present invention provides genetically modified seed. Progeny, variants, and mutants of the regenerated plants are also included within the scope of the invention, provided that these parts comprise the genetic modification. Further provided is a processed plant product or byproduct that retains the genetic modification, including for example, soymeal.

The methods provided herein may be used for modification of any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

Vegetables include, but are not limited to, tomatoes, lettuce, green beans, lima beans, peas, and members of the genus Cucumis such as cucumber, cantaloupe, and musk melon. Ornamentals include, but are not limited to, azalea, hydrangea, hibiscus, roses, tulips, daffodils, petunias, carnation, poinsettia, and chrysanthemum. Preferably, plants of the present invention are crop plants (for example, maize, sorghum, wheat, sunflower, tomato, crucifers, peppers, potato, cotton, rice, soybean, sugarbeet, sugarcane, tobacco, barley, oilseed rape, etc.).

The methods provided herein can also be used to genetically modify any prokaryotic species, including but not limited to, archaea and bacteria (e.g., Bacillus sp., Klebsiella sp. Streptomyces sp., Rhizobium sp., Escherichia sp., Pseudomonas sp., Salmonella sp., Shigella sp., Vibrio sp., Yersinia sp., Mycoplasma sp., Agrobacterium, Lactobacillus sp.).

The methods provided herein can be used to genetically modify any eukaryotic species or cells therefrom, including but not limited to animals (e.g., mammals, insects, fish, birds, and reptiles), fungi, amoeba, algae, and yeast. In some embodiments, the cell that is modified by the presently disclosed methods include cells of hematopoietic origin, such as immune cells (i.e., a cell of the innate or adaptive immune system) including but not limited to B cells, T cells, natural killer (NK) cells, pluripotent stem cells, induced pluripotent stem cells, chimeric antigen receptor T (CAR-T) cells, monocytes, macrophages, and dendritic cells.

Cells that have been modified may be introduced into an organism. These cells could have originated from the same organism (e.g., person) in the case of autologous cellular transplants, wherein the cells are modified in an ex vivo approach. In some embodiments, the cells originated from another organism within the same species (e.g., another person) in the case of allogeneic cellular transplants.

### XIII. Kits

Some aspects of this disclosure provide kits comprising a deaminase of the invention. In certain embodiments, the disclosure provides kits comprising a fusion protein comprising a deaminase of the invention and a DNA-binding polypeptide (e.g., an RNA-guided, DNA-binding polypeptide, such as an RGN polypeptide, for example a nuclease-inactive or nickase RGN), and, optionally, a linker positioned between the DNA-binding polypeptide and the deaminase. In addition, in some embodiments, the kit comprises suitable reagents, buffers, and/or instructions for using the fusion protein, e.g., for in vitro or in vivo DNA or RNA editing. In some embodiments, the kit comprises instructions regarding the design and use of suitable gRNAs for targeted editing of a nucleic acid sequence.

The article “a” and “an” are used herein to refer to one or more than one (i.e., to at least one) of the grammatical object of the article. By way of example, “a polypeptide” means one or more polypeptides.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated herein by reference.

Although the foregoing invention has been described in some detail by way of illustration and example for purposes of clarity of understanding, it will be obvious that certain changes and modifications may be practiced within the scope of the appended claims.

Non-limiting embodiments include:

1. A polypeptide comprising an amino acid sequence selected from the group consisting of:
a) an amino acid sequence having at least 90% sequence identity to any one of SEQ ID NOs: 2 and 7-12; and
b) an amino acid sequence having at least 95% sequence identity to SEQ ID NO: 4 or 6; wherein said polypeptide has deaminase activity.

2. An isolated polypeptide comprising an amino acid sequence selected from the group consisting of:
a) an amino acid sequence having at least 90% sequence identity to any one of SEQ ID NOs: 2 and 7-12; and
b) an amino acid sequence having at least 95% sequence identity to SEQ ID NO: 4 or 6; wherein said polypeptide has deaminase activity.

3. The polypeptide of embodiment 1 or 2, comprising an amino acid sequence having at least 95% sequence identity to any one of SEQ ID NOs: 2 and 7-12.

4. The polypeptide of embodiment 1 or 2, comprising an amino acid sequence having 100% sequence identity to any one of SEQ ID NOs: 2, 4, and 6-12.

5. A nucleic acid molecule comprising a polynucleotide encoding a deaminase polypeptide, wherein the deaminase is encoded by a nucleotide sequence selected from the group consisting of:
a) a nucleotide sequence having at least 80% sequence identity to any one of SEQ ID NOs: 114-119;
b) a nucleotide sequence having at least 95% sequence identity to any one of SEQ ID NOs: 109, 111, and 113
c) a nucleotide sequence encoding an amino acid sequence having at least 90% sequence identity to any one of SEQ ID NOs: 2 and 7-12; and
d) a nucleotide sequence encoding an amino acid sequence having at least 95% sequence identity to SEQ ID NO: 4 or 6.

6. An isolated nucleic acid molecule comprising a polynucleotide encoding a deaminase polypeptide, wherein the deaminase is encoded by a nucleotide sequence selected from the group consisting of:
a) a nucleotide sequence having at least 80% sequence identity to any one of SEQ ID NOs: 114-119;
b) a nucleotide sequence having at least 95% sequence identity to any one of SEQ ID NOs: 109, 111, and 113
c) a nucleotide sequence encoding an amino acid sequence having at least 90% sequence identity to any one of SEQ ID NOs: 2 and 7-12; and
d) a nucleotide sequence encoding an amino acid sequence having at least 95% sequence identity to SEQ ID NO: 4 or 6.

7. The nucleic acid molecule of embodiment 5 or 6, wherein the deaminase is encoded by a nucleotide sequence that has at least 90% sequence identity to any one of SEQ ID NOs: 114-119.

8. The nucleic acid molecule of embodiment 5 or 6, wherein the deaminase is encoded by a nucleotide sequence that has at least 95% sequence identity to any one of SEQ ID NOs: 114-119.

9. The nucleic acid molecule of embodiment 5 or 6, wherein the deaminase is encoded by a nucleotide sequence that has 100% sequence identity to any one of SEQ ID NOs: 109, 111, and 113-119.

10. The nucleic acid molecule of embodiment 5 or 6, wherein the deaminase polypeptide has an amino acid sequence having at least 95% sequence identity to any one of SEQ ID NOs: 2 and 7-12.

11. The nucleic acid molecule of embodiment 5 or 6, wherein the deaminase polypeptide has an amino acid sequence having 100% sequence identity to any one of SEQ ID NOs: 2, 4, and 6-12.

12. The nucleic acid molecule of any one of embodiments 5-11, wherein said nucleic acid molecule further comprises a heterologous promoter operably linked to said polynucleotide.

13. A vector comprising said nucleic acid molecule of any one of embodiments 5-12.

14. A cell comprising said nucleic acid molecule of any one of embodiments 5-12 or said vector of embodiment 13.

15. The cell of embodiment 14, wherein the cell is a prokaryotic cell.

16. The cell of embodiment 14, wherein the cell is a eukaryotic cell.

17. The cell of embodiment 16, wherein the eukaryotic cell is a mammalian cell.

18. The cell of embodiment 17, wherein the mammalian cell is a human cell.

19. The cell of embodiment 18, wherein the human cell is an immune cell.

20. The cell of embodiment 19, wherein the immune cell is a stem cell.

21. The cell of embodiment 20, wherein the stem cell is an induced pluripotent stem cell.

22. The cell of embodiment 16, wherein the eukaryotic cell is an insect or avian cell.

23. The cell of embodiment 16, wherein the eukaryotic cell is a fungal cell.

24. The cell of embodiment 16, wherein the eukaryotic cell is a plant cell.

25. A plant comprising the cell of embodiment 24.

26. A seed comprising the cell of embodiment 24.

27. A pharmaceutical composition comprising a pharmaceutically acceptable carrier and the polypeptide of any one of embodiments 1-4, the nucleic acid molecule of any one of embodiments 5-12, the vector of embodiment 13, or the cell of any one of embodiments 14-24.

28. The pharmaceutical composition of embodiment 27, wherein the pharmaceutically acceptable carrier is heterologous to said polypeptide or said nucleic acid molecule.

29. The pharmaceutical composition of embodiment 27 or 28, wherein the pharmaceutically acceptable carrier is not naturally-occurring.

30. A method for making a deaminase comprising culturing the cell of any one of embodiments 14-24 under conditions in which the deaminase is expressed.

31. A method for making a deaminase comprising introducing into a cell the nucleic acid molecule of any of embodiments 5-12 or a vector of embodiment 13 and culturing the cell under conditions in which the deaminase is expressed.

32. The method of embodiment 30 or 31, further comprising purifying said deaminase.

33. A fusion protein comprising a DNA-binding polypeptide and a deaminase having an amino acid sequence selected from the group consisting of:
a) an amino acid sequence having at least 90% sequence identity to any one of SEQ ID NOs: 2 and 7-12; and
b) an amino acid sequence having at least 95% sequence identity to SEQ ID NO: 4 or 6.

34. The fusion protein of embodiment 33, wherein said deaminase has at least 95% sequence identity to any one of SEQ ID NOs: 2 and 7-12.

35. The fusion protein of embodiment 33, wherein said deaminase has 100% sequence identity to any one of SEQ ID NOs: 2, 4, and 6-12.

36. The fusion protein of any one of embodiments 33-35, wherein the deaminase is a cytosine deaminase.

37. The fusion protein of any one of embodiments 33-36, wherein the DNA-binding polypeptide is a meganuclease, a zinc finger fusion protein, or a TALEN; or a variant of a meganuclease, a zinc finger fusion protein, or a TALEN, wherein the nuclease activity has been reduced or inhibited.

38. The fusion protein of any one of embodiments 33-36, wherein the DNA-binding polypeptide is an RNA-guided, DNA-binding polypeptide.

39. The fusion protein of embodiment 38, wherein the RNA-guided, DNA-binding polypeptide is an RNA-guided nuclease (RGN) polypeptide.

40. The fusion protein of embodiment 39, wherein the RGN is a Type II CRISPR-Cas polypeptide.

41. The fusion protein of embodiment 39, wherein the RGN is a Type V CRISPR-Cas polypeptide.

42. The fusion protein of any one of embodiments 39-41, wherein the RGN is an RGN nickase.

43. The fusion protein of embodiment 42, wherein the RGN nickase has an inactive RuvC domain.

44. The fusion protein of any one of embodiments 39-41, wherein the RGN is a nuclease-inactive RGN.

45. The fusion protein of embodiment 39, wherein the RGN has an amino acid sequence having at least 90% sequence identity to any one of the RGN sequences in Table 1.

46. The fusion protein of embodiment 39, wherein the RGN has an amino acid sequence having at least 95% sequence identity to any one of the RGN sequences in Table 1.

47. The fusion protein of embodiment 39, wherein the RGN has an amino acid sequence of any one of the RGN sequences in Table 1.

48. The fusion protein of embodiment 39, wherein the RGN has an amino acid sequence having at least 90% sequence identity to any one of SEQ ID NOs: 74, 82, 87, 106, and 107.

49. The fusion protein of embodiment 39, wherein the RGN has an amino acid sequence having at least 95% sequence identity to any one of SEQ ID NOs: 74, 82, 87, 106, and 107.

50. The fusion protein of embodiment 39, wherein the RGN has an amino acid sequence of any one of SEQ ID NOs: 74, 82, 87, 106, and 107.

51. The fusion protein of embodiment 42, wherein the RGN nickase has an amino acid sequence having at least 90% sequence identity to any one of SEQ ID NOs: 75 and 88-98.

52. The fusion protein of embodiment 42, wherein the RGN nickase has an amino acid sequence having at least 95% sequence identity to any one of SEQ ID NOs: 75 and 88-98.

53. The fusion protein of embodiment 42, wherein the RGN nickase has an amino acid sequence having any one of SEQ ID NOs: 75 and 88-98.

54. The fusion protein of any of embodiments 33-53, wherein the fusion protein further comprises at least one nuclear localization signal (NLS).

55. The fusion protein of any one of embodiments 33-54, wherein the deaminase is fused to the amino terminus of the DNA-binding polypeptide.

56. The fusion protein of any one of embodiments 33-54, wherein the deaminase is fused to the carboxyl terminus of the DNA-binding polypeptide.

57. The fusion protein of any one of embodiments 33-56, wherein the fusion protein further comprises a linker sequence between said DNA-binding polypeptide and said deaminase.

58. The fusion protein of embodiment 57, wherein said linker sequence has an amino acid sequence set forth as SEQ ID NO: 78 or 79.

59. The fusion protein of any one of embodiments 33-58, wherein said fusion protein further comprises a uracil stabilizing protein (USP).

60. The fusion protein of embodiment 59, wherein said USP has the sequence set forth as SEQ ID NO: 81.

61. The fusion protein of embodiment 59 or 60, wherein said fusion protein further comprises a linker sequence between said USP and said deaminase or said DNA-binding polypeptide.

62. The fusion protein of embodiment 61, wherein said linker sequence between said USP and said deaminase or said DNA-binding polypeptide has an amino acid sequence set forth as SEQ ID NO: 120.

63. The fusion protein of embodiment 33, wherein said fusion protein has an amino acid sequence of any one of SEQ ID NOs: 67, 68, 146, and 147.

64. A nucleic acid molecule comprising a polynucleotide encoding a fusion protein comprising a DNA-binding polypeptide and a deaminase, wherein the deaminase is encoded by a nucleotide sequence selected from the group consisting of:
a) a nucleotide sequence having at least 80% sequence identity to any one of SEQ ID NOs: 114-119;
b) a nucleotide sequence having at least 95% sequence identity to any one of SEQ ID NOs: 109, 111, and 113;
c) a nucleotide sequence encoding an amino acid sequence having at least 90% sequence identity to any one of SEQ ID NOs: 2 and 7-12; and
d) a nucleotide sequence encoding an amino acid sequence having at least 95% sequence identity to SEQ ID NO: 4 or 6.

65. The nucleic acid molecule of embodiment 64, wherein said deaminase is encoded by a nucleotide sequence has at least 90% sequence identity to any one of SEQ ID NOs: 114-119.

66. The nucleic acid molecule of embodiment 64, wherein said deaminase is encoded by a nucleotide sequence has at least 95% sequence identity to any one of SEQ ID NOs: 114-119.

67. The nucleic acid molecule of embodiment 64, wherein said deaminase nucleotide sequence has 100% sequence identity to any one of SEQ ID NOs: 109, 111, and 113-119.

68. The nucleic acid molecule of embodiment 64, wherein said deaminase nucleotide sequence encodes an amino acid sequence having at least 95% sequence identity to any one of SEQ ID NOs: 2 and 7-12.

69. The nucleic acid molecule of embodiment 64, wherein said deaminase nucleotide sequence encodes an amino acid sequence having 100% sequence identity to any one of SEQ ID NOs: 2, 4, and 6-12.

70. The nucleic acid molecule of any one of embodiments 64-69, wherein the deaminase is a cytosine deaminase.

71. The nucleic acid molecule of any one of embodiments 64-70, wherein the DNA-binding polypeptide is a meganuclease, a zinc finger fusion protein, or a TALEN; or a variant of a meganuclease, a zinc finger fusion protein, or a TALEN, wherein the nuclease activity has been reduced or inhibited.

72. The nucleic acid molecule of any one of embodiments 64-70, wherein the DNA-binding polypeptide is an RNA-guided, DNA-binding polypeptide.

73. The nucleic acid molecule of embodiment 72, wherein the RNA-guided, DNA-binding polypeptide is an RNA-guided nuclease (RGN) polypeptide.

74. The nucleic acid molecule of embodiment 73, wherein the RGN is a Type II CRISPR-Cas polypeptide.

75. The nucleic acid molecule of embodiment 73, wherein the RGN is a Type V CRISPR-Cas polypeptide.

76. The nucleic acid molecule of any one of embodiments 73-75, wherein the RGN is an RGN nickase.

77. The nucleic acid molecule of embodiment 76, wherein said RGN nickase has an inactive RuvC domain.

78. The nucleic acid molecule of any one of embodiments 73-75, wherein the RGN is a nuclease-inactive RGN.

79. The nucleic acid molecule of embodiment 73, wherein the RGN has an amino acid sequence having at least 90% sequence identity to any one of the RGN sequences in Table 1.

80. The nucleic acid molecule of embodiment 73, wherein the RGN has an amino acid sequence having at least 95% sequence identity to any one of the RGN sequences in Table 1.

81. The nucleic acid molecule of embodiment 73, wherein the RGN has an amino acid sequence of any one of the RGN sequences in Table 1.

82. The nucleic acid molecule of embodiment 73, wherein the RGN has an amino acid sequence having at least 90% sequence identity to any one of SEQ ID NOs: 74, 82, 87, 106, and 107.

83. The nucleic acid molecule of embodiment 73, wherein the RGN has an amino acid sequence having at least 95% sequence identity to any one of SEQ ID NOs: 74, 82, 87, 106, and 107.

84. The nucleic acid molecule of embodiment 73, wherein the RGN has an amino acid sequence of any one of SEQ ID NOs: 74, 82, 87, 106, and 107.

85. The nucleic acid molecule of embodiment 76, wherein the RGN nickase has an amino acid sequence having at least 90% sequence identity to any one of SEQ ID NOs: 75 and 88-98.

86. The nucleic acid molecule of embodiment 76, wherein the RGN nickase has an amino acid sequence having at least 95% sequence identity to any one of SEQ ID NOs: 75 and 88-98.

87. The nucleic acid molecule of embodiment 76, wherein the RGN nickase has an amino acid sequence having any one of SEQ ID NOs: 75 and 88-98.

88. The nucleic acid molecule of any of embodiments 64-87, wherein the polynucleotide encoding the fusion protein is operably linked at its 5′ end to a promoter.

89. The nucleic acid molecule of any of embodiments 64-88, wherein the polynucleotide encoding the fusion protein is operably linked at its 3′ end to a terminator.

90. The nucleic acid molecule of any of embodiments 64-89, wherein the fusion protein comprises one or more nuclear localization signals.

91. The nucleic acid molecule of any of embodiments 64-90, wherein the fusion protein is codon optimized for expression in a eukaryotic cell.

92. The nucleic acid molecule of any of embodiments 64-90, wherein the fusion protein is codon optimized for expression in a prokaryotic cell.

93. The nucleic acid molecule of any one of embodiments 64-92, wherein the deaminase is fused to the amino terminus of the DNA-binding polypeptide.

94. The nucleic acid molecule of any one of embodiments 64-92, wherein the deaminase is fused to the carboxyl terminus of the DNA-binding polypeptide.

95. The nucleic acid molecule of any one of embodiments 64-94, wherein the fusion protein further comprises a linker sequence between said DNA-binding polypeptide and said deaminase.

96. The nucleic acid molecule of embodiment 95, wherein said linker sequence has an amino acid sequence set forth as SEQ ID NO: 78 or 79.

97. The nucleic acid molecule of any one of embodiments 64-96, wherein said fusion protein further comprises a uracil stabilizing protein (USP).

98. The nucleic acid molecule of embodiment 97, wherein said USP has the sequence set forth as SEQ ID NO: 81.

99. The nucleic acid molecule of embodiment 97 or 98, wherein said fusion protein further comprises a linker sequence between said USP and said deaminase or said DNA-binding polypeptide.

100. The nucleic acid molecule of embodiment 99, wherein said linker sequence between said USP and said deaminase or said DNA-binding polypeptide has an amino acid sequence set forth as SEQ ID NO: 120.

101. The nucleic acid molecule of embodiment 64, wherein said fusion protein has an amino acid sequence set forth as any one of SEQ ID NOs: 67, 68, 146, and 147.

102. A vector comprising the nucleic acid molecule of any one of embodiments 64-101.

103. The vector of embodiment 102, further comprising at least one nucleotide sequence encoding a guide RNA (gRNA) capable of hybridizing to a target sequence.

104. The vector of embodiment 103, wherein the gRNA is a single guide RNA.

105. The vector of embodiment 103, wherein the gRNA is a dual guide RNA.

106. A cell comprising the fusion protein of any of embodiments 33-63.

107. The cell of embodiment 106, wherein the cell further comprises a guide RNA (gRNA).

108. The cell of embodiment 107, wherein the gRNA is a single guide RNA.

109. The cell of embodiment 107, wherein the gRNA is a dual guide RNA.

110. A cell comprising the nucleic acid molecule of any one of embodiments 64-101.

111. A cell comprising the vector of any one of embodiments 102-105.

112. The cell of any one of embodiments 106-111, wherein the cell is a prokaryotic cell.

113. The cell of any one of embodiments 106-111, wherein the cell is a eukaryotic cell.

114. The cell of embodiment 113, wherein the eukaryotic cell is a mammalian cell.

115. The cell of embodiment 114, wherein the mammalian cell is a human cell.

116. The cell of embodiment 115, wherein the human cell is an immune cell.

117. The cell of embodiment 116, wherein the immune cell is a stem cell.

118. The cell of embodiment 117, wherein the stem cell is an induced pluripotent stem cell.

119. The cell of embodiment 113, wherein the eukaryotic cell is an insect or avian cell.

120. The cell of embodiment 113, wherein the eukaryotic cell is a fungal cell.

121. The cell of embodiment 113, wherein the eukaryotic cell is a plant cell.

122. A plant comprising the cell of embodiment 121.

123. A seed comprising the cell of embodiment 121.

124. A pharmaceutical composition comprising a pharmaceutically acceptable carrier and the fusion protein of any one of embodiments 33-63, the nucleic acid molecule of any one of embodiments 64-101, the vector of any one of embodiments 102-105, or the cell of any one of embodiments 114-118.

125. A method for making a fusion protein comprising culturing the cell of any one of embodiments 106-121 under conditions in which the fusion protein is expressed.

126. A method for making a fusion protein comprising introducing into a cell the nucleic acid molecule of any of embodiments 64-101 or a vector of any one of embodiments 102-105 and culturing the cell under conditions in which the fusion protein is expressed.

127. The method of embodiment 125 or 126, further comprising purifying said fusion protein.

128. A method for making an RGN fusion ribonucleoprotein complex, comprising introducing into a cell the nucleic acid molecule of any one of embodiments 72-87 and a nucleic acid molecule comprising an expression cassette encoding a guide RNA (gRNA), or the vector of any of embodiments 103-105, and culturing the cell under conditions in which the fusion protein and the gRNA are expressed and form an RGN fusion ribonucleoprotein complex.

129. The method of embodiment 128, further comprising purifying said RGN fusion ribonucleoprotein complex.

130. A system for modifying a target DNA molecule comprising a target DNA sequence, said system comprising:
a) a fusion protein or a nucleotide sequence encoding said fusion protein, wherein said fusion protein comprises an RNA-guided nuclease polypeptide (RGN) and a deaminase, wherein the deaminase has an amino acid sequence selected from the group consisting of:
   i) an amino acid sequence having at least 90% sequence identity to any one of SEQ ID NOs: 2 and 7-12; and
   ii) an amino acid sequence having at least 95% sequence identity to SEQ ID NO: 4 or 6; and
b) one or more guide RNAs capable of hybridizing to said target DNA sequence or one or more nucleotide sequences encoding the one or more guide RNAs (gRNAs); and
wherein the one or more guide RNAs are capable of forming a complex with the fusion protein in order to direct said fusion protein to bind to said target DNA sequence and modify the target DNA molecule.

131. The system of embodiment 130, wherein said deaminase has an amino acid sequence having at least 95% sequence identity to any one of SEQ ID NOs: 2 and 7-12.

132. The system of embodiment 130, wherein said deaminase has an amino acid sequence having 100% sequence identity to any one of SEQ ID NOs: 2, 4, and 6-12.

133. The system of any one of embodiments 130-132, wherein at least one of said nucleotide sequence encoding the one or more guide RNAs and said nucleotide sequence encoding the fusion protein is operably linked to a promoter.

134. The system of any one of embodiments 130-133, wherein the target DNA sequence is a eukaryotic target DNA sequence.

135. The system of any one of embodiments 130-134, wherein the target DNA sequence is located adjacent to a protospacer adjacent motif (PAM) that is recognized by the RGN.

136. The system of any one of embodiments 130-135, wherein the target DNA molecule is within a cell.

137. The system of embodiment 136, wherein the cell is a eukaryotic cell.

138. The system of embodiment 137, wherein the eukaryotic cell is a plant cell.

139. The system of embodiment 137, wherein the eukaryotic cell is a mammalian cell.

140. The system of embodiment 139, wherein the mammalian cell is a human cell.

141. The system of embodiment 140, wherein the human cell is an immune cell.

142. The system of embodiment 141, wherein the immune cell is a stem cell.

143. The system of embodiment 142, wherein the stem cell is an induced pluripotent stem cell.

144. The system of embodiment 137, wherein the eukaryotic cell is an insect cell.

145. The system of embodiment 136, wherein the cell is a prokaryotic cell.

146. The system of any one of embodiments 130-145, wherein the RGN of the fusion protein is a Type II CRISPR-Cas polypeptide.

147. The system of any one of embodiments 130-145, wherein the RGN of the fusion protein is a Type V CRISPR-Cas polypeptide.

148. The system of any one of embodiments 130-145, wherein the RGN of the fusion protein has an amino acid sequence having at least 90% sequence identity to any one of the RGN sequences in Table 1.

149. The system of any one of embodiments 130-145, wherein the RGN of the fusion protein has an amino acid sequence having at least 95% sequence identity to any one of the RGN sequences in Table 1.

150. The system of any one of embodiments 130-145, wherein the RGN of the fusion protein has an amino acid sequence of any one of the RGN sequences in Table 1.

151. The system of any one of embodiments 130-145, wherein the RGN of the fusion protein has an amino acid sequence having at least 90% sequence identity to any one of SEQ ID NOs: 74, 82, 87, 106, and 107.

152. The system of any one of embodiments 130-145, wherein the RGN of the fusion protein has an amino acid sequence having at least 95% sequence identity to any one of SEQ ID NOs: 74, 82, 87, 106, and 107.

153. The system of any one of embodiments 130-145, wherein the RGN of the fusion protein has an amino acid sequence of any one of SEQ ID NOs: 74, 82, 87, 106, and 107.

154. The system of any one of embodiments 130-145, wherein the RGN of the fusion protein is an RGN nickase.

155. The system of embodiment 154, wherein the RGN nickase has an inactive RuvC domain.

156. The system of embodiment 154 or 155, wherein the RGN nickase has an amino acid sequence having at least 90% sequence identity to any one of SEQ ID NOs: 75 and 88-98.

157. The system of embodiment 154 or 155, wherein the RGN nickase has an amino acid sequence having at least 95% sequence identity to any one of SEQ ID NOs: 75 and 88-98.

158. The system of embodiment 154 or 155, wherein the RGN nickase is any one of SEQ ID NOs: 75 and 88-98.

159. The system of any one of embodiments 130-145, wherein the RGN of the fusion protein is a nuclease-inactive RGN.

160. The system of any of embodiments 130-159, wherein the fusion protein comprises one or more nuclear localization signals.

161. The system of any one of embodiments 130-160, wherein the deaminase is fused to the amino terminus of the DNA-binding polypeptide.

162. The system of any one of embodiments 130-160, wherein the deaminase is fused to the carboxyl terminus of the DNA-binding polypeptide.

163. The system of any one of embodiments 130-162, wherein the fusion protein further comprises a linker sequence between said DNA-binding polypeptide and said deaminase.

164. The system of embodiment 163, wherein said linker sequence has an amino acid sequence set forth as SEQ ID NO: 78 or 79.

165. The system of any one of embodiments 130-164, wherein said fusion protein further comprises a uracil stabilizing protein (USP).

166. The system of embodiment 165, wherein said USP has the sequence set forth as SEQ ID NO: 81.

167. The system of embodiment 165 or 166, wherein said fusion protein further comprises a linker sequence between said USP and said deaminase or said DNA-binding polypeptide.

168. The system of embodiment 167, wherein said linker sequence between said USP and said deaminase or said DNA-binding polypeptide has an amino acid sequence set forth as SEQ ID NO: 120.

169. The system of embodiment 130, wherein the fusion protein has an amino acid sequence set forth as any one of SEQ ID NOs: 67, 68, 146, and 147.

170. The system of any one of embodiments 130-169, wherein the fusion protein is codon optimized for expression in a eukaryotic cell.

171. The system of any of embodiments 130-170, wherein the one or more nucleotide sequences encoding the one or more guide RNAs and the nucleotide sequence encoding a fusion protein are located on one vector.

172. A ribonucleoprotein complex comprising said at least one guide RNA and said fusion protein of the system of any one of embodiments 130-171.

173. A cell comprising the system of any one of embodiments 130-171 or the ribonucleoprotein complex of embodiment 172.

174. The cell of embodiment 173, wherein the cell is a prokaryotic cell.

175. The cell of embodiment 173, wherein the cell is a eukaryotic cell.

176. The cell of embodiment 175, wherein the eukaryotic cell is a mammalian cell.

177. The cell of embodiment 176, wherein the mammalian cell is a human cell.

178. The cell of embodiment 177, wherein the human cell is an immune cell.

179. The cell of embodiment 178, wherein the immune cell is a stem cell.

180. The cell of embodiment 179, wherein the stem cell is an induced pluripotent stem cell.

181. The cell of embodiment 175, wherein the eukaryotic cell is an insect or avian cell.

182. The cell of embodiment 175, wherein the eukaryotic cell is a fungal cell.

183. The cell of embodiment 175, wherein the eukaryotic cell is a plant cell.

184. A plant comprising the cell of embodiment 183.

185. A seed comprising the cell of embodiment 183.

186. A pharmaceutical composition comprising a pharmaceutically acceptable carrier and the system of any one of embodiments 130-171, the ribonucleoprotein complex of embodiment 172, or the cell of any one of embodiments 175-180.

187. A method for modifying a target DNA molecule comprising a target DNA sequence, said method comprising delivering a system according to any one of embodiments 130-171 or a ribonucleoprotein complex of claim 172 to said target DNA molecule or a cell comprising the target DNA molecule.

188. The method of embodiment 187, wherein said modified target DNA molecule comprises a C>N mutation of at least one nucleotide within the target DNA molecule, wherein N is A, G, or T.

189. The method of embodiment 188, wherein said modified target DNA molecule comprises an C>T mutation of at least one nucleotide within the target DNA molecule.

190. The method of embodiment 188, wherein said modified target DNA molecule comprises an C>G mutation of at least one nucleotide within the target DNA molecule.

191. A method for modifying a target DNA molecule comprising a target sequence, said method comprising:
a) assembling an RGN-deaminase ribonucleotide complex in vitro by combining:
   i) one or more guide RNAs capable of hybridizing to the target DNA sequence; and
   ii) a fusion protein comprising an RNA-guided nuclease polypeptide (RGN), and at least one deaminase, wherein the deaminase has an amino acid sequence selected from the group consisting of:
      I) an amino acid sequence having at least 90% sequence identity to any one of SEQ ID NOs: 2 and 7-12; and
      II) an amino acid sequence having at least 95% sequence identity to SEQ ID NO: 4 or 6;
under conditions suitable for formation of the RGN-deaminase ribonucleotide complex; and
b) contacting said target DNA molecule or a cell comprising said target DNA molecule with the in vitro-assembled RGN-deaminase ribonucleotide complex;
wherein the one or more guide RNAs hybridize to the target DNA sequence, thereby directing said fusion protein to bind to said target DNA sequence and modification of the target DNA molecule occurs.

192. The method of embodiment 191, wherein said deaminase has an amino acid sequence having at least 95% sequence identity to any one of SEQ ID NOs: 2 and 7-12.

193. The method of embodiment 191, wherein said deaminase has an amino acid sequence having 100% sequence identity to any one of SEQ ID NOs: 2, 4, and 6-12.

194. The method of any one of embodiments 191-193, wherein said modified target DNA molecule comprises a C>N mutation of at least one nucleotide within the target DNA molecule, wherein N is A, G, or T.

195. The method of embodiment 194, wherein said modified target DNA molecule comprises a C>T mutation of at least one nucleotide within the target DNA molecule.

196. The method of embodiment 194, wherein said modified target DNA molecule comprises a C>G mutation of at least one nucleotide within the target DNA molecule.

197. The method of any one of embodiments 191-196, wherein the RGN of the fusion protein is a Type II CRISPR-Cas polypeptide.

198. The method of any of embodiments 191-196, wherein the RGN of the fusion protein is a Type V CRISPR-Cas polypeptide.

199. The method of any one of embodiments 191-198, wherein the RGN of the fusion protein has an amino acid sequence having at least 90% sequence identity to any one of the RGN sequences in Table 1.

200. The method of any one of embodiments 191-198, wherein the RGN of the fusion protein has an amino acid sequence having at least 95% sequence identity to any one of the RGN sequences in Table 1.

201. The method of any one of embodiments 191-198, wherein the RGN of the fusion protein has an amino acid sequence of any one of the RGN sequences in Table 1.

202. The method of any one of embodiments 191-198, wherein the RGN of the fusion protein has an amino acid sequence having at least 90% sequence identity to any one of SEQ ID NOs: 74, 82, 87, 106, and 107.

203. The method of any one of embodiments 191-198, wherein the RGN of the fusion protein has an amino acid sequence having at least 95% sequence identity to any one of SEQ ID NOs: 74, 82, 87, 106, and 107.

204. The method of any one of embodiments 191-198, wherein the RGN of the fusion protein has an amino acid sequence of any one of SEQ ID NOs: 74, 82, 87, 106, and 107.

205. The method of any of embodiments 191-198, wherein the RGN of the fusion protein is an RGN nickase.

206. The method of embodiment 205, wherein said RGN nickase has an inactive RuvC domain.

207. The method of embodiment 205 or 206, wherein said RGN nickase has an amino acid sequence having at least 90% sequence identity to any one of SEQ ID NOs: 75 and 88-98.

208. The method of embodiment 205 or 206, wherein said RGN nickase has an amino acid sequence having at least 95% sequence identity to any one of SEQ ID NOs: 75 and 88-98.

209. The method of embodiment 205 or 206, wherein the RGN nickase is any one of SEQ ID NOs: 75 and 88-98.

210. The method of any one of embodiments 191-198, wherein the RGN of the fusion protein is a nuclease-inactive RGN.

211. The method of any of embodiments 191-210, wherein the fusion protein comprises one or more nuclear localization signals.

212. The method of any one of embodiments 191-211, wherein the deaminase is fused to the amino terminus of the DNA-binding polypeptide.

213. The method of any one of embodiments 191-211, wherein the deaminase is fused to the carboxyl terminus of the DNA-binding polypeptide.

214. The method of any one of embodiments 191-213, wherein the fusion protein further comprises a linker sequence between said DNA-binding polypeptide and said deaminase.

215. The method of embodiment 214, wherein said linker sequence has an amino acid sequence set forth as SEQ ID NO: 78 or 79.

216. The method of any one of embodiments 191-215, wherein said fusion protein further comprises a uracil stabilizing protein (USP).

217. The method of embodiment 216, wherein said USP has the sequence set forth as SEQ ID NO: 81.

218. The method of embodiment 216 or 217, wherein said fusion protein further comprises a linker sequence between said USP and said deaminase or said DNA-binding polypeptide.

219. The method of embodiment 218, wherein said linker sequence between said USP and said deaminase or said DNA-binding polypeptide has an amino acid sequence set forth as SEQ ID NO: 120.

220. The method of embodiment 191, wherein said fusion protein has an amino acid sequence set forth as any one of SEQ ID NOs: 67, 68, 146, and 147.

221. The method of any one of embodiments 191-220, wherein said target DNA sequence is a eukaryotic target DNA sequence.

222. The method of any of embodiments 191-221, wherein said target DNA sequence is located adjacent to a protospacer adjacent motif (PAM).

223. The method of any of embodiments 191-222, wherein the target DNA molecule is within a cell.

224. The method of embodiment 223, wherein the cell is a eukaryotic cell.

225. The method of embodiment 224, wherein the eukaryotic cell is a plant cell.

226. The method of embodiment 224, wherein the eukaryotic cell is a mammalian cell.

227. The method of embodiment 226, wherein the mammalian cell is a human cell.

228. The method of embodiment 227, wherein the human cell is an immune cell.

229. The method of embodiment 228, wherein the immune cell is a stem cell.

230. The method of embodiment 229, wherein the stem cell is an induced pluripotent stem cell.

231. The method of embodiment 224, wherein the eukaryotic cell is an insect cell.

232. The method of embodiment 223, wherein the cell is a prokaryotic cell.

233. The method of any one of embodiments 223-232, further comprising selecting a cell comprising said modified DNA molecule.

234. A cell comprising a modified target DNA sequence according to the method of embodiment

233.

235. The cell of embodiment 234, wherein the cell is a eukaryotic cell.

236. The cell of embodiment 235, wherein the eukaryotic cell is a plant cell.

237. A plant comprising the cell of embodiment 236.

238. A seed comprising the cell of embodiment 236.

239. The cell of embodiment 235, wherein the eukaryotic cell is a mammalian cell.

240. The cell of embodiment 239, wherein the mammalian cell is a human cell.

241. The cell of embodiment 240, wherein the human cell is an immune cell.

242. The cell of embodiment 241, wherein the immune cell is a stem cell.

243. The cell of embodiment 242, wherein the stem cell is an induced pluripotent stem cell.

244. The cell of embodiment 235, wherein the eukaryotic cell is an insect cell.

245. The cell of embodiment 234, wherein the cell is a prokaryotic cell.

246. A pharmaceutical composition comprising the cell of any one of embodiments 239-243, and a pharmaceutically acceptable carrier.

247. A method for producing a genetically modified cell with a correction in a causal mutation for a genetically inherited disease, the method comprising introducing into the cell:
a) a fusion protein or a polynucleotide encoding said fusion protein, wherein said fusion protein comprises an RNA-guided nuclease polypeptide (RGN) and a deaminase, wherein the deaminase has an amino acid sequence selected from the group consisting of:
   i) an amino acid sequence having at least 90% sequence identity to any one of SEQ ID NOs: 2 and 7-12; and
   ii) an amino acid sequence having at least 95% sequence identity to SEQ ID NO: 4 or 6;

and
b) one or more guide RNAs (gRNA) capable of hybridizing to a target DNA sequence, or a polynucleotide encoding said gRNA;
whereby the fusion protein and gRNA target to the genomic location of the causal mutation and modify the genomic sequence to remove the causal mutation.

248. The method of embodiment 247, wherein said polynucleotide encoding the fusion protein is operably linked to a promoter active in said cell.

249. The method of embodiment 247 or 248, wherein said polynucleotide encoding the gRNA is operably linked to a promoter active in said cell.

250. The method of any one of embodiments 247-249, wherein said deaminase has an amino acid sequence having at least 95% sequence identity to any one of SEQ ID NOs: 2 and 7-12.

251. The method of any one of embodiments 247-249, wherein said deaminase has an amino acid sequence having 100% sequence identity to any one of SEQ ID NOs: 2, 4, and 6-12.

252. The method of any one of embodiments 247-251, wherein the RGN of the fusion protein is a Type II CRISPR-Cas polypeptide.

253. The method of any of embodiments 247-251, wherein the RGN of the fusion protein is a Type V CRISPR-Cas polypeptide.

254. The method of any one of embodiments 247-253, wherein the RGN of the fusion protein has an amino acid sequence having at least 90% sequence identity to any one of the RGN sequences in Table 1.

255. The method of any of embodiments 247-253, wherein the RGN of the fusion protein has an amino acid sequence having at least 95% sequence identity to any one of the RGN sequences in Table 1.

256. The method of any one of embodiments 247-253, wherein the RGN of the fusion protein has an amino acid sequence of any one of the RGN sequences in Table 1.

257. The method of any of embodiments 247-253, wherein the RGN of the fusion protein is an RGN nickase.

258. The method of embodiment 257, wherein said RGN nickase has an inactive RuvC domain.

259. The method of embodiment 257 or 258, wherein the RGN nickase has an amino acid sequence having at least 90% sequence identity to any one of SEQ ID NOs: 75 and 88-98.

260. The method of embodiment 257 or 258, wherein the RGN nickase has an amino acid sequence having at least 95% sequence identity to any one of SEQ ID NOs: 75 and 88-98.

261. The method of embodiment 257 or 258, wherein the RGN nickase is any one of SEQ ID NOs: 75 and 88-98.

262. The method of any one of embodiments 247-253, wherein the RGN of the fusion protein is a nuclease-inactive RGN.

263. The method of any of embodiments 247-262, wherein the fusion protein comprises one or more nuclear localization signals.

264. The method of any one of embodiments 247-263, wherein the deaminase is fused to the amino terminus of the DNA-binding polypeptide.

265. The method of any one of embodiments 247-263, wherein the deaminase is fused to the carboxyl terminus of the DNA-binding polypeptide.

266. The method of any one of embodiments 247-265, wherein the fusion protein further comprises a linker sequence between said DNA-binding polypeptide and said deaminase.

267. The method of embodiment 266, wherein said linker sequence has an amino acid sequence set forth as SEQ ID NO: 78 or 79.

268. The method of any one of embodiments 247-267, wherein said fusion protein further comprises a uracil stabilizing protein (USP).

269. The method of embodiment 268, wherein said USP has the sequence set forth as SEQ ID NO: 81.

270. The method of embodiment 268 or 269, wherein said fusion protein further comprises a linker sequence between said USP and said deaminase or said DNA-binding polypeptide.

271. The method of embodiment 270, wherein said linker sequence between said USP and said deaminase or said DNA-binding polypeptide has an amino acid sequence set forth as SEQ ID NO: 120.

272. The method of any one of embodiments 247-249, wherein said fusion protein has an amino acid sequence set forth as any one of SEQ ID NOs: 67, 68, 146, and 147.

273. The method of any one of embodiments 247-272, wherein the genome modification comprises introducing a C>T mutation of at least one nucleotide within the target DNA sequence.

274. The method of any one of embodiments 247-272, wherein the genome modification comprises introducing a C>G mutation of at least one nucleotide within the target DNA sequence.

275. The method of any of embodiments 247-274, wherein the cell is an animal cell.

276. The method of embodiment 275, wherein the animal cell is a mammalian cell.

277. The method of embodiment 276, wherein the cell is derived from a dog, cat, mouse, rat, rabbit, horse, sheep, goat, cow, pig, or human.

278. The method of any one of embodiments 247-277, wherein the correction of the causal mutation comprises correcting a nonsense mutation.

279. The method of any one of embodiments 247-278, wherein the genetically inherited disease is a disease listed in Table 23.

280. The method of embodiment 279, wherein the gRNA further comprises a spacer sequence that targets any one of SEQ ID NOs: 122-144, or the complement thereof.

281. A composition comprising:
a) a fusion protein comprising a DNA-binding polypeptide and a cytosine deaminase, or a nucleic acid molecule encoding the fusion protein; and
b) a second cytosine deaminase or a nucleic acid molecule encoding the second deaminase, wherein the second deaminase has an amino acid sequence selected from the group consisting of:
   i) an amino acid sequence having at least 90% sequence identity to any one of SEQ ID NOs: 2 and 7-12; and
   ii) an amino acid sequence having at least 95% sequence identity to SEQ ID NO: 4 or 6.

282. The composition of embodiment 281, wherein said second cytosine deaminase has at least 95% sequence identity to any one of SEQ ID NOs: 2 and 7-12.

283. The composition of embodiment 281, wherein said second cytosine deaminase has 100% sequence identity to any one of SEQ ID NOs: 2, 4, and 6-12.

284. The composition of any one of embodiments 281-283, wherein the first cytosine deaminase has an amino acid sequence selected from the group consisting of:
a) an amino acid sequence having at least 90% sequence identity to any one of SEQ ID NOs: 2 and 7-12; and
b) an amino acid sequence having at least 95% sequence identity to SEQ ID NO: 4 or 6.

285. The composition of any one of embodiments 281-284, wherein the first cytosine deaminase has at least 95% sequence identity to any one of SEQ ID NOs: 2 and 7-12.

286. The composition of any one of embodiments 281-284, wherein the first cytosine deaminase has 100% sequence identity to any one of SEQ ID NOs: 2, 4, and 6-12.

287. The composition of any one of embodiments 281-286, wherein the DNA-binding polypeptide is a meganuclease, a zinc finger fusion protein, or a TALEN; or a variant of a meganuclease, a zinc finger fusion protein, or a TALEN, wherein the nuclease activity has been reduced or inhibited.

288. The composition of any one of embodiments 281-286, wherein the DNA-binding polypeptide is an RNA-guided, DNA-binding polypeptide.

289. The composition of embodiment 288, wherein the RNA-guided, DNA-binding polypeptide is an RNA-guided nuclease (RGN) polypeptide.

290. The composition of embodiment 289, wherein the RGN is an RGN nickase.

291. The composition of embodiment 289, wherein the RGN is a nuclease-inactive RGN.

292. A vector comprising a nucleic acid molecule encoding a fusion protein and a nucleic acid molecule encoding a second cytosine deaminase, wherein said fusion protein comprises a DNA-binding polypeptide and a first cytosine deaminase, and wherein said second cytosine deaminase has an amino acid sequence selected from the group consisting of:
a) an amino acid sequence having at least 90% sequence identity to any one of SEQ ID NOs: 2 and 7-12; and
b) an amino acid sequence having at least 95% sequence identity to SEQ ID NO: 4 or 6.

293. The vector of embodiment 292, wherein said second cytosine deaminase has at least 95% sequence identity to any one of SEQ ID NOs: 2 and 7-12.

294. The vector of embodiment 292, wherein said second cytosine deaminase has 100% sequence identity to any one of SEQ ID NOs: 2, 4, and 6-12.

295. The vector of any one of embodiments 292-294, wherein the first cytosine deaminase has an amino acid sequence selected from the group consisting of:
a) an amino acid sequence having at least 90% sequence identity to any one of SEQ ID NOs: 2 and 7-12; and
b) an amino acid sequence having at least 95% sequence identity to SEQ ID NO: 4 or 6.

296. The vector of any one of embodiments 292-294, wherein the first cytosine deaminase has at least 95% sequence identity to any one of SEQ ID NOs: 2 and 7-12.

297. The vector of any one of embodiments 292-294, wherein the first cytosine deaminase has 100% sequence identity to any one of SEQ ID NOs: 2, 4, and 6-12.

298. The vector of any one of embodiments 292-297, wherein the DNA-binding polypeptide is a meganuclease, a zinc finger fusion protein, or a TALEN; or a variant of a meganuclease, a zinc finger fusion protein, or a TALEN, wherein the nuclease activity has been reduced or inhibited.

299. The vector of any one of embodiments 292-297, wherein the DNA-binding polypeptide is an RNA-guided, DNA-binding polypeptide.

300. The vector of embodiment 299, wherein the RNA-guided, DNA-binding polypeptide is an RNA-guided nuclease (RGN) polypeptide.

301. The vector of embodiment 300, wherein the RGN is an RGN nickase.

302. The vector of embodiment 300, wherein the RGN is a nuclease-inactive RGN.

303. A cell comprising the vector of any one of embodiments 292-302.

304. A cell comprising:
a) a fusion protein comprising a DNA-binding polypeptide and a first cytosine deaminase; or a nucleic acid molecule encoding the fusion protein; and
b) a second cytosine deaminase or a nucleic acid molecule encoding the second cytosine deaminase, wherein the second cytosine deaminase has an amino acid sequence selected from the group consisting of:
   i) an amino acid sequence having at least 90% sequence identity to any one of SEQ ID NOs: 2 and 7-12; and
   ii) an amino acid sequence having at least 95% sequence identity to SEQ ID NO: 4 or 6.
305. The cell of embodiment 304, wherein said second cytosine deaminase has at least 95% sequence identity to any one of SEQ ID NOs: 2 and 7-12.
306. The cell of embodiment 304, wherein said second cytosine deaminase has 100% sequence identity to any one of SEQ ID NOs: 2, 4, and 6-12.
307. The cell of any one of embodiments 304-306, wherein the first cytosine deaminase has an amino acid sequence selected from the group consisting of:
a) an amino acid sequence having at least 90% sequence identity to any one of SEQ ID NOs: 2 and 7-12; and
b) an amino acid sequence having at least 95% sequence identity to SEQ ID NO: 4 or 6.

308. The cell of any one of embodiments 304-306, wherein the first cytosine deaminase has at least 95% sequence identity to any one of SEQ ID NOs: 2 and 7-12.

309. The cell of any one of embodiments 304-306, wherein the first cytosine deaminase has 100% sequence identity to any one of SEQ ID NOs: 2, 4, and 6-12.

310. The cell of any one of embodiments 304-309, wherein the DNA-binding polypeptide is a meganuclease, a zinc finger fusion protein, or a TALEN; or a variant of a meganuclease, a zinc finger fusion protein, or a TALEN, wherein the nuclease activity has been reduced or inhibited.

311. The cell of any one of embodiments 304-309, wherein the DNA-binding polypeptide is an RNA-guided, DNA-binding polypeptide.

312. The cell of embodiment 311, wherein the RNA-guided, DNA-binding polypeptide is an RNA-guided nuclease (RGN) polypeptide.

313. The cell of embodiment 312, wherein the RGN is an RGN nickase.

314. The cell of embodiment 312, wherein the RGN is a nuclease-inactive RGN.

315. A pharmaceutical composition comprising a pharmaceutically acceptable carrier and the composition of any one of embodiments 281-291, the vector of any one of embodiments 292-302, or the cell of any one of embodiments 303-314.

316. A method for treating a disease, said method comprising administering to a subject in need thereof the fusion protein of any one of embodiments 33-63, the nucleic acid molecule of any one of embodiments 64-101, the vector of any one of embodiments 102-105 and 292-302, the cell of any one of embodiments 113-118, 239-243, and 303-314, the system of any one of embodiments 130-171, the ribonucleoprotein complex of embodiment 172, the composition of any one of embodiments 281-291, or the pharmaceutical composition of any one of embodiments 124, 186, 246, and 315.

317. The method of embodiment 316, wherein said disease is associated with a causal mutation and said pharmaceutical composition corrects said causal mutation.

318. The method of embodiment 316 or 317, wherein said disease is a disease a disease listed in Table 23.

319. Use of the fusion protein of any one of embodiments 33-63, the nucleic acid molecule of any one of embodiments 64-101, the vector of any one of embodiments 102-105 and 292-302, the cell of any one of embodiments 113-118, 239-243, and 303-314, the system of any one of embodiments 130-171, the ribonucleoprotein complex of embodiment 172, or the composition of any one of embodiments 281-291 for the treatment of a disease in a subject.

320. The use of embodiment 319, wherein said disease is associated with a causal mutation and said treating comprises correcting said causal mutation.

321. The use of embodiment 319 or 320, wherein said disease is a disease listed in Table 23.

322. Use of the fusion protein of any one of embodiments 33-63, the nucleic acid molecule of any one of embodiments 64-101, the vector of any one of embodiments 102-105 and 292-302, the cell of any one of embodiments 113-118, 239-243, and 303-314, the system of any one of embodiments 130-171, the ribonucleoprotein complex of embodiment 172, or the composition of any one of embodiments 281-291 for the manufacture of a medicament useful for treating a disease.

323. The use of embodiment 322, wherein said disease is associated with a causal mutation and an effective amount of said medicament corrects said causal mutation.

324. The use of embodiment 322 or 323, wherein said disease is a disease listed in Table 23.

The following examples are offered by way of illustration and not by way of limitation.

### EXPERIMENTAL

### Example 1: Demonstration of C Base Editing in Mammalian Cells

Deaminases set forth as SEQ ID NOs: 1, 3, and 5 were truncated from both termini and the truncated deaminases are set forth as SEQ ID NOs: 2, 4, and 6. Cytosine deaminases derived from bacteria are set forth as SEQ ID NOs: 7-12.

TABLE 2
Deaminase sequences

| Deaminase | SEQ ID |
|---|---|
| APG09980 | 1 |
| APG09980.1 | 2 |
| APG05840 | 3 |
| APG05840.1 | 4 |
| APG00868 | 5 |
| APG00868.1 | 6 |
| APG30125 | 7 |
| APG30126 | 8 |
| APG30127 | 9 |
| APG30128 | 10 |
| APG30129 | 11 |
| APG30130 | 12 |

To determine if the deaminases of Table 2 are able to perform cytosine base editing in mammalian cells, each deaminase was operably fused to an RGN nickase to produce a fusion protein. Residues predicted to deactivate the RuvC domain of the RGN APG07433.1 (SEQ ID NO: 74; described in PCT Publication No. WO 2019/236566, incorporated by reference herein) were identified and the RGN was modified to a nickase variant (nAPG07433.1; SEQ ID NO: 75). A nickase variant of an RGN is referred to herein as “nRGN”. It should be understood that any nickase variant of an RGN may be used to produce a fusion protein of the invention.

Deaminase and nRGN nucleotide sequences codon optimized for mammalian expression were synthesized as fusion proteins with an N-terminal nuclear localization tag and cloned into the pTwist CMV (Twist Biosciences) expression plasmid. Each fusion protein comprises, starting at the amino terminus, the SV40 NLS (SEQ ID NO: 76) operably linked at the C-terminal end to 3× FLAG Tag (SEQ ID NO: 77), operably linked at the C-terminal end to a deaminase, operably linked at the C-terminal end to a peptide linker (L16 or L32; set forth as SEQ ID NO: 78 or 79, respectively), operably linked at the C-terminal end to the nRGN (for example, nAPG07433.1, which is SEQ ID NO: 75), finally operably linked at the C-terminal end to the nucleoplasmin NLS (SEQ ID NO: 80). Table 3 shows the fusion proteins produced and tested for activity. All fusion proteins comprise at least one NLS and a 3× FLAG Tag, as described above. The APG05840.1-nAPG07433.1-USP2 fusion protein in Table 3 further comprises a uracil stabilizing protein USP2 (set forth as SEQ ID NO: 81) between the nRGN and the nucleoplasmin NLS. This fusion protein also comprises a peptide linker having the sequence set forth as SEQ ID NO: 120 between nAPG07433.1 and the USP2.

TABLE 3
Fusion protein sequences with N-terminus SV40 NLS,
3X FLAG Tag and C-terminus Nucleoplasmin NLS

| Fusion Protein | SEQ ID |
|---|---|
| APG09980-L16- | 13 |
| nAPG07433.1 | |
| APG09980.1-L16- | 14 |
| nAPG07433.1 | |
| APG05840-L16- | 15 |
| nAPG07433.1 | |
| APG05840.1-L16- | 16 |
| nAPG07433.1 | |
| APG00868-L16- | 17 |
| nAPG07433.1 | |
| APG00868.1-L16- | 18 |
| nAPG07433.1 | |
| APG30125- | 19 |
| nAPG07433.1 | |
| APG30126- | 20 |
| nAPG07433.1 | |
| APG30127- | 21 |
| nAPG07433.1 | |
| APG30128- | 22 |
| nAPG07433.1 | |
| APG30129- | 23 |
| nAPG07433.1 | |
| APG05840.1- | 24 |
| nAPG07433.1- | |
| USP2 | |
| APG30130- | 145 |
| nAPG07433.1 | |

Expression plasmids comprising an expression cassette encoding a sgRNA were also produced. Human genomic target sequences and the sgRNA sequences for guiding the fusion proteins to the genomic targets are indicated in Table 4.

TABLE 4
Guide RNA sequences

| | | | Forward | Reverse |
|---|---|---|---|---|
| | Target | sgRNA | Primer for | Primer for |
| sgRNA ID | sequence | sequence | amplification | amplification |
| SGN000169 | 25 | 34 | 43 | 52 |
| SGN000173 | 26 | 35 | 44 | 53 |
| SGN000929 | 27 | 36 | 45 | 54 |
| SGN001101 | 28 | 37 | 46 | 55 |
| SGN000927 | 29 | 38 | 47 | 56 |
| SGN000143 | 30 | 39 | 48 | 57 |
| SGN000186 | 31 | 40 | 49 | 58 |
| SGN000194 | 32 | 41 | 50 | 59 |
| SGN000930 | 33 | 42 | 51 | 60 |

500 ng of plasmid comprising an expression cassette comprising a coding sequence for each fusion protein shown in Table 3 and 500 ng of plasmid comprising an expression cassette encoding an sgRNA shown in Table 4 were co-transfected into HEK293FT cells at 75-90% confluency in 24-well plates using Lipofectamine 2000 reagent (Life Technologies). Cells were then incubated at 370 C for 72 h. Following incubation, genomic DNA was then extracted using NucleoSpin 96 Tissue (Macherey-Nagel) following the manufacturer's protocol. The genomic region flanking the targeted genomic site was PCR amplified using the primers in Table 4 and products were purified using ZR-96 DNA Clean and Concentrator (Zymo Research) following the manufacturer's protocol. The purified PCR products were then sent for Next Generation Sequencing on Illumina MiSeq (2×250). Results were analyzed for INDEL formation or introduction of % specific cytosine mutations.

Table 5 shows all cytosine base editing for each combination of a fusion protein from Table 3 and a guide RNA from Table 4. Tables 6-11 show the specific nucleotide mutation profile for select exemplary samples. The position of each nucleotide in the target sequence was determined. “C17” indicates, for example, a cytosine at position 17 of the target sequence. The position of each nucleotide in the target sequence was determined by numbering the first nucleotide in the target sequence closest to the PAM as position 1, and the position number increases in the 3′ direction away from the PAM sequence. Tables 6-11 also show which nucleotide the cytosine was changed to, and at what rate. For example, Table 6 shows that for the APG30127-nAPG07433.1 fusion protein, the cytosine at position 17 was mutated to a thymidine at a rate of 0.2%.

TABLE 5
Estimate of base editing for each deaminase
and linker combination tested

| | | % Mutated |
|---|---|---|
| Construct | Target | Reads |
| APG09980_L16_nAPG07433.1 | SGN001101 | 29.18% |
| APG09980_L16_nAPG07433.1 | SGN000929 | 24.48% |
| APG09980_L16_nAPG07433.1 | SGN000169 | 24.79% |
| APG09980_L16_nAPG07433.1 | SGN000173 | 17.71% |
| APG09980_L16_nAPG07433.1 | SGN000143 | 11.20% |
| APG09980_L16_nAPG07433.1 | SGN000930 | 25.25% |
| APG09980.1_L16_nAPG07433.1 | SGN001101 | 30.52% |
| APG09980.1_L16_nAPG07433.1 | SGN000929 | 25.95% |
| APG09980.1_L16_nAPG07433.1 | SGN000169 | 24.05% |
| APG09980.1_L16_nAPG07433.1 | SGN000173 | 22.25% |
| APG09980.1_L16_nAPG07433.1 | SGN000143 | 9.70% |
| APG09980.1_L16_nAPG07433.1 | SGN000930 | 23.80% |
| APG05840_L16_nAPG07433.1 | SGN001101 | 24.30% |
| APG05840_L16_nAPG07433.1 | SGN000929 | 27.67% |
| APG05840_L16_nAPG07433.1 | SGN000169 | 20.53% |
| APG05840_L16_nAPG07433.1 | SGN000173 | 11.38% |
| APG05840_L16_nAPG07433.1 | SGN000143 | 15.13% |
| APG05840_L16_nAPG07433.1 | SGN000930 | 22.38% |
| APG05840.1_L16_nAPG07433.1 | SGN001101 | 23.83% |
| APG05840.1_L16_nAPG07433.1 | SGN000929 | 22.34% |
| APG05840.1_L16_nAPG07433.1 | SGN000169 | 30.22% |
| APG05840.1_L16_nAPG07433.1 | SGN000173 | 20.44% |
| APG05840.1_L16_nAPG07433.1 | SGN000143 | 10.97% |
| APG05840.1_L16_nAPG07433.1 | SGN000930 | 22.22% |
| APG05840.1-L16- | SGN001101 | 12.67% |
| nAPG07433.1-USP2 | | |
| APG05840.1-L16- | SGN000929 | 11.14% |
| nAPG07433.1-USP2 | | |
| APG05840.1-L16- | SGN000169 | 22.56% |
| nAPG07433.1-USP2 | | |
| APG05840.1-L16- | SGN000173 | 11.36% |
| nAPG07433.1-USP2 | | |
| APG05840.1-L16- | SGN000930 | 13.11% |
| nAPG07433.1-USP2 | | |
| APG05840.1-L16- | SGN000143 | 6.25% |
| nAPG07433.1-USP2 | | |
| APG00868_L16_nAPG07433.1 | SGN001101 | 24.40% |
| APG00868_L16_nAPG07433.1 | SGN000929 | 20.62% |
| APG00868_L16_nAPG07433.1 | SGN000169 | 16.36% |
| APG00868_L16_nAPG07433.1 | SGN000173 | 13.22% |
| APG00868_L16_nAPG07433.1 | SGN000143 | 8.36% |
| APG00868_L16_nAPG07433.1 | SGN000930 | 14.86% |
| APG00868.1_L16_nAPG07433.1 | SGN001101 | 20.36% |
| APG00868.1_L16_nAPG07433.1 | SGN000929 | 14.64% |
| APG00868.1_L16_nAPG07433.1 | SGN000169 | 22.39% |
| APG00868.1_L16_nAPG07433.1 | SGN000173 | 18.37% |
| APG00868.1_L16_nAPG07433.1 | SGN000143 | 6.33% |
| APG00868.1_L16_nAPG07433.1 | SGN000930 | 12.29% |
| APG30125-L32-nAPG07433.1 | SGN001101 | 2.72% |
| APG30125-L32-nAPG07433.1 | SGN000929 | 7.39% |
| APG30125-L32-nAPG07433.1 | SGN000169 | 6.89% |
| APG30125-L32-nAPG07433.1 | SGN000173 | 3.54% |
| APG30125-L32-nAPG07433.1 | SGN000930 | 6.32% |
| APG30125-L32-nAPG07433.1 | SGN000143 | 0% |
| APG30126-L32-nAPG07433.1 | SGN000930 | 1.38% |
| APG30126-L32-nAPG07433.1 | SGN000143 | 0% |
| APG30126-L32-nAPG07433.1 | SGN000186 | 0% |
| APG30126-L32-nAPG07433.1 | SGN000194 | 0% |
| APG30126-L32-nAPG07433.1 | SGN000927 | 0% |
| APG30126-L32-nAPG07433.1 | SGN000139 | 0% |
| APG30127-L32-nAPG07433.1 | SGN000930 | 6.22% |
| APG30127-L32-nAPG07433.1 | SGN000143 | 2.46% |
| APG30127-L32-nAPG07433.1 | SGN000186 | 14.26% |
| APG30127-L32-nAPG07433.1 | SGN000194 | 9.53% |
| APG30127-L32-nAPG07433.1 | SGN000927 | 3.26% |
| APG30127-L32-nAPG07433.1 | SGN000139 | 0.00% |
| APG30128-L32-nAPG07433.1 | SGN000930 | 2.03% |
| APG30128-L32-nAPG07433.1 | SGN000143 | 0.00% |
| APG30128-L32-nAPG07433.1 | SGN000186 | 0.00% |
| APG30128-L32-nAPG07433.1 | SGN000194 | 0.00% |
| APG30128-L32-nAPG07433.1 | SGN000927 | 0.00% |
| APG30128-L32-nAPG07433.1 | SGN000139 | 0.00% |
| APG30129-L32-nAPG07433.1 | SGN000930 | 3.47% |
| APG30129-L32-nAPG07433.1 | SGN000143 | 2.02% |
| APG30129-L32-nAPG07433.1 | SGN000186 | 11.40% |
| APG30129-L32-nAPG07433.1 | SGN000194 | 5.92% |
| APG30129-L32-nAPG07433.1 | SGN000927 | 0.00% |
| APG30129-L32-nAPG07433.1 | SGN000139 | 0.00% |
| APG30130-L32-nAPG07433.1 | SGN000930 | 3.14% |
| APG30130-L32-nAPG07433.1 | SGN000143 | 0.00% |
| APG30130-L32-nAPG07433.1 | SGN000186 | 2.78% |
| APG30130-L32-nAPG07433.1 | SGN000194 | 3.29% |
| APG30130-L32-nAPG07433.1 | SGN000927 | 0.00% |

TABLE 6
C > N Editing Rate using deaminase
APG30127 and guide SGN000930

| | | SGN000930 | | |
|---|---|---|---|---|
| | | C17 | C19 | C22 |
| APG30127 | A | 0 | 0 | 0 |
| | G | 5.3 | 0.4 | 0 |
| | T | 0.2 | 0.4 | 0.2 |

APG30127 showed predominantly C>G conversions at position C17.

TABLE 7
C > N Editing Rate using deaminase APG30127 and guide SGN000186

| | | SGN000186 | | | | | | | | | |
|---|---|---|---|---|---|---|---|---|---|---|---|
| | | C10 | C12 | C17 | C23 | C24 | C29 | C3 | C4 | C5 | C7 |
| APG30127 | A | 0.2 | 0 | 0.5 | 0.1 | 0 | 0 | 0 | 0 | 0 | 0.1 |
| | G | 0.1 | 0 | 0.6 | 0 | 0.1 | 0.2 | 0 | 0 | 0 | 0 |
| | T | 1.6 | 3.3 | 2 | 0.1 | 0 | 0.2 | 0.1 | 0.1 | 0 | 0 |

APG30127 shows C>T conversions at multiple positions, including C10, C12, and C17. At position C17, there is also C>G and C>A conversions.

TABLE 8
C > N Editing Rate using deaminase APG30129 and guide SGN000186

| | | SGN000186 | | | | | | | | | |
|---|---|---|---|---|---|---|---|---|---|---|---|
| | | C10 | C12 | C17 | C23 | C24 | C29 | C3 | C4 | C5 | C7 |
| APG30129 | A | 0.1 | 0 | 0.3 | 0.1 | 0.2 | 0.2 | 0 | 0 | 0 | 0 |
| | G | 0.2 | 0.2 | 0.5 | 0 | 0 | 0 | 0 | 0 | 0 | 0 |
| | T | 1.1 | 3.5 | 1.2 | 0.1 | 0 | 0.1 | 0 | 0 | 0.2 | 0.3 |

APG30129 shows C>T conversions at positions C10, C12, and C17.

TABLE 9
C > N Editing Rate using deaminase APG30125 and guide SGN000186

| | | SGN000186 | | | | | | | | | |
|---|---|---|---|---|---|---|---|---|---|---|---|
| | | C10 | C12 | C17 | C23 | C24 | C29 | C3 | C4 | C5 | C7 |
| APG30125 | A | 0.4 | 0.1 | 0.3 | 0 | 0 | 0.1 | 0 | 0 | 0 | 0 |
| | G | 0.2 | 0 | 0.1 | 0 | 0 | 0.1 | 0 | 0 | 0 | 0 |
| | T | 1 | 2.9 | 1.3 | 0.2 | 0.1 | 0.2 | 0 | 0 | 0.1 | 0 |

APG30125 shows C>T conversions at positions C10, C12, and C17. There is less C>G and C>A conversions at all positions than the APG30129 and APG30127 samples.

TABLE 10
C > N Editing Rate using deaminase APG05840.1-
L16- nAPG07433.1-USP2 and guide SGN000169

| | | SGN000169 | | | | | | | | | |
|---|---|---|---|---|---|---|---|---|---|---|---|
| | | C4 | C9 | C13 | C15 | C18 | C20 | C23 | C26 | C27 | C29 |
| APG05840.1-L16- | A | 0 | 0 | 0.1 | 0.2 | 0.1 | 0.1 | 0 | 0.2 | 0 | 0 |
| nAPG07433.1- | G | 0 | 0 | 0.1 | 0.8 | 0 | 0 | 0 | 0 | 0 | 0 |
| USP2 | T | 0 | 5.3 | 16.3 | 20.2 | 2.1 | 11.3 | 7.4 | 0 | 0 | 0 |

Truncated APG05840 (APG05840.1) was tested with a 16 amino acid linker (L16) and a uracil stabilizing protein (USP2). This construct showed high levels of specific C>T editing at several positions in target SGN000169, including C9, C13, C15, C20 and C23. This demonstrates that the shortened deaminase and the shorter linker can still be used to generate site specific single nucleotide edits.

TABLE 11
C > N Editing Rate using deaminase APG05840.1-
L16- nAPG07433.1-USP2 and guide SGN000173

| | | SGN000173 | | | | | | | | | | |
|---|---|---|---|---|---|---|---|---|---|---|---|---|
| | | C1 | C3 | C4 | C7 | C8 | C10 | C11 | C17 | C2 | C20 | C29 |
| APG05840.1-L16- | A | 0 | 0 | 0 | 0 | 0.1 | 0 | 0 | 0.3 | 0 | 0 | 0 |
| nAPG07433.1-USP2 | G | 0 | 0 | 0 | 0 | 0 | 0.1 | 0.1 | 0 | 0 | 0 | 0 |
| | T | 0 | 0 | 0 | 3.4 | 6.4 | 5 | 1.9 | 9.3 | 0 | 0.7 | 0 |

Truncated APG05840 (APG05840.1) was tested with a 16 amino acid linker (L16) and a uracil stabilizing protein (USP2). This construct showed high levels of specific C>T editing at several positions in target SGN000173, including C7, C8, C10, and C17.

### Example 2: Off-Target, RGN-independent Cytosine Deaminase Driven Effects Assay

In order to determine if there are any mutational effects on ssDNA by the cytosine deaminase, an RGN-independent, off-target mutation assay was performed. Residues predicted to deactivate the RuvC and HNH domains of the RGN APG09298 (SEQ ID NO: 82; described in PCT Publication No. WO 2021/217002, which is herein incorporated by reference in its entirety) were identified and the RGN was modified to a dead variant (dAPG09298; SEQ ID NO: 83).

The dRGN nucleotide sequence codon optimized for expression was synthesized as fusion proteins with an N-terminal nuclear localization tag and cloned into the pTwist CMV (Twist Biosciences) expression plasmid. This dRGN fusion protein comprises, starting at the amino terminus, the SV40 NLS (SEQ ID NO: 76) operably linked at the C-terminal end to 3× FLAG Tag (SEQ ID NO: 77), operably linked at the C-terminal end to the dRGN (for example, dAPG09298, which is SEQ ID NO: 83), finally operably linked at the C-terminal end to the Nucleoplasmin NLS (SEQ ID NO: 80) to make NLS-dAPG09298-NLS (SEQ ID NO: 84). This construct is used to create ssDNA in an R-loop at a location unrelated to the target sequence being edited by the cytosine deaminase base editor.

Expression plasmids comprising an expression cassette encoding a sgRNA were also produced. Human genomic target sequences and the sgRNA sequences for guiding the fusion proteins to the genomic targets are indicated in Table 4.

TABLE 12
Off-target guide RNA sequences

| | | | Forward | Reverse |
|---|---|---|---|---|
| | Target | sgRNA | Primer for | Primer for |
| sgRNA ID | sequence | sequence | amplification | amplification |
| SGN001165 | 61 | 62 | 63 | 64 |

500 ng of plasmid comprising an expression cassette comprising a coding sequence for a fusion protein shown in Table 3 and 500 ng of plasmid comprising an expression cassette encoding an sgRNA shown in Table 4 and 500 ng of plasmid comprising an expression cassette comprising a coding sequence for NLS-dAPG09298-NLS and 500 ng of plasmid comprising an expression cassette encoding an sgRNA for dAPG09298 shown in Table 12 were co-transfected into HEK293FT cells at 75-90% confluency in 24-well plates using Lipofectamine 2000 reagent (Life Technologies). Cells were then incubated at 370 C for 72 h. Following incubation, genomic DNA was then extracted using NucleoSpin 96 Tissue (Macherey-Nagel) following the manufacturer's protocol. The genomic region flanking the targeted genomic site was PCR amplified using the primers in Table 4 or Table 12 and products were purified using ZR-96 DNA Clean and Concentrator (Zymo Research) following the manufacturer's protocol. The purified PCR products were then sent for Next Generation Sequencing on Illumina MiSeq (2×250). Results were analyzed for INDEL formation or specific cytosine mutation. On-target results were those identified by the amplicon in Table 4. RGN-independent, off-target results were those identified by the amplicon in Table 12.

TABLE 13
Deaminase-driven RGN-independent, off-target
effects with APG05840-nAPG07433.1-USP2

| | | % Mutated reads at off |
|---|---|---|
| | % Mutated reads | target site for deaminase- |
| | at On Target site | driven effects (SGN001165) |
| SGN001101 | 19.7% | 0.99% |
| SGN000929 | 24% | 0% |
| SGN000169 | 25% | 0% |
| SGN000173 | 18.24% | 0% |
| SGN000930 | 20.24% | 0% |
| SGN000143 | 18.13% | 0% |

The intended on-target site for each sample showed high levels of cytosine specific mutations. The off-target site, bound by dAPG09298 at SGN001165 showed no mutated reads in five out of six samples. One target tested showed low mutation rates at the off-target location with 0.99% f reads having mutations. These may be RGN-independent, deaminase-driven mutation effects, but could also be sequencing errors because of the low rate in the sample.

### Example 3: Demonstration of C>G Base Editing in Mammalian Cells

These studies assessed whether the orientation of the deaminase and RGN to each other in a fusion protein affects the type of base editing that occurs by the resultant C base editor.

Residues predicted to deactivate the RuvC domain of the RGN APG07433.1 (SEQ ID NO: 74; PCT Publication No. WO 2019/236566, incorporated by reference herein) were identified and the RGN was modified to a nickase variant (nAPG07433.1; SEQ ID NO: 75).

Deaminase (APG09980 and APG05840; set forth as SEQ ID NOs: 1 and 3, respectively) and nAPG07433.1 nucleotide sequences codon optimized for expression were synthesized as fusion proteins with an N-terminal nuclear localization tag and cloned into the pTwist CMV (Twist Biosciences) expression plasmid. Each fusion protein comprises, starting at the amino terminus, the SV40 NLS (SEQ ID NO: 76) operably linked at the C-terminal end to 3× FLAG Tag (SEQ ID NO: 77), operably linked at the C-terminal end to the nRGN-deaminase, deaminase-nRGN-USP2, or deaminase-nRGN fusion protein, connected by a peptide linker (SEQ ID NO: 79), finally operably linked at the C-terminal end to the Nucleoplasmin NLS (SEQ ID NO: 80). Table 14 shows the fusion proteins produced and tested for activity. All fusion proteins comprise at least one NLS and a 3× FLAG Tag, as described above. The APG09980-nAPG07433.1-USP2 and APG05840.1-nAPG07433.1-USP2 fusion protein in Table 14 further comprise a uracil stabilizing protein USP2 (set forth as SEQ ID NO: 81) between the nRGN and the nucleoplasmin NLS. The APG09980-nAPG07433.1-USP2 and APG05840.1-nAPG07433.1-USP2 fusion proteins also comprises a peptide linker having the sequence set forth as SEQ ID NO: 120 between nAPG07433.1 and the USP2.

TABLE 14
Fusion protein sequences with N-terminus SV40 NLS,
3X FLAG Tag and C-terminus Nucleoplasmin NLS

| Fusion Protein | SEQ ID |
|---|---|
| nAPG07433.1- | 65 |
| APG09980 | |
| nAPG07433.1- | 66 |
| APG05840 | |
| APG09980- | 67 |
| nAPG07433.1- | |
| USP2 | |
| APG05840- | 68 |
| nAPG07433.1- | |
| USP2 | |
| APG05840- | 69 |
| nAPG07433.1 | |

Expression plasmids comprising an expression cassette encoding a sgRNA were also produced. Human genomic target sequences and the sgRNA sequences for guiding the fusion proteins to the genomic targets are indicated in Table 15.

TABLE 15
guide RNA sequences

| | | | Forward | Reverse |
|---|---|---|---|---|
| | Target | sgRNA | Primer for | Primer for |
| sgRNA ID | sequence | sequence | amplification | amplification |
| SGN000930 | 33 | 42 | 51 | 60 |
| SGN000928 | 70 | 71 | 72 | 73 |

500 ng of plasmid comprising an expression cassette comprising a coding sequence for a fusion protein shown in Table 14 and 500 ng of plasmid comprising an expression cassette encoding for an sgRNA shown in Table 15 were co-transfected into HEK293FT cells at 75-90% confluency in 24-well plates using Lipofectamine 2000 reagent (Life Technologies). Cells were then incubated at 370 C for 72 h. Following incubation, genomic DNA was then extracted using NucleoSpin 96 Tissue (Macherey-Nagel) following the manufacturer's protocol. The genomic region flanking the targeted genomic site was PCR amplified using the primers in Table 15 and products were purified using ZR-96 DNA Clean and Concentrator (Zymo Research) following the manufacturer's protocol. The purified PCR products were then sent for Next Generation Sequencing on Illumina MiSeq (2×250). Results were analyzed for INDEL formation or specific cytosine mutation.

Tables 16-20 show cytosine base editing for each combination of a fusion protein from Table 14 and a guide RNA from Table 15. The position of each nucleotide in the target sequence was determined. “C16” indicates, for example, a cytosine at position 16 of the target sequence. The position of each nucleotide in the target sequence was determined by numbering the first nucleotide in the target sequence closest to the PAM as position 1, and the position number increases in the 3′ direction away from the PAM sequence. Tables 16-20 also show which nucleotide the cytosine was changed to, and at what rate. For example, Table 16 shows that for the APG05840-nAPG07433.1-USP2 fusion protein, the cytosine at position 16 was mutated to a thymidine at a rate of 11%.

TABLE 16
C > N Editing Rate using deaminase APG05840 and guide SGN000928

| | | SGN000928 | | | | | | | | | |
|---|---|---|---|---|---|---|---|---|---|---|---|
| | | C2 | C3 | C4 | C7 | C11 | C16 | C18 | C21 | C24 | C27 |
| APG05840- | A | 0 | 0 | 0 | 0 | 0.1 | 0.4 | 0.2 | 0.1 | 0 | 0 |
| nAPG07433.1-USP2 | G | 0 | 0 | 0 | 0 | 0.4 | 1.4 | 0 | 0.6 | 0 | 0 |
| | T | 0 | 0 | 0 | 0.8 | 3.3 | 11 | 2.1 | 11 | 0.3 | 2.2 |

In the orientation with the deaminase on the N-terminus in the full construct APG05840-nAPG07433.1-USP2, high levels of specific C>T conversion are evident at positions C16 and C21 in SGN000928.

TABLE 17
C > N Editing Rate using deaminase
APG05840 and guide SGN000930

| | | SGN000930 | | |
|---|---|---|---|---|
| | | C17 | C19 | C22 |
| APG05840- | A | 0 | 0 | 0 |
| nAPG07433.1- | G | 4.9 | 0 | 0 |
| USP2 | T | 17 | 2.8 | 6.9 |

In the orientation with the deaminase on the N-terminus in the full construct APG05840-nAPG07433.1-USP2, high levels of specific C>T conversion are evident at positions C17 and C22 in SGN000930. Some C>G conversion is evident at position C17.

TABLE 18
C > N Editing Rate using deaminase APG05840 and guide SGN000928

| | | SGN000928 | | | | | | | | | |
|---|---|---|---|---|---|---|---|---|---|---|---|
| | | C2 | C3 | C4 | C7 | C11 | C16 | C18 | C21 | C24 | C27 |
| nAPG07433.1- | A | 0 | 0 | 0 | 0 | 0.1 | 0.5 | 0 | 0.2 | 0 | 0 |
| APG05840 | G | 0 | 0 | 0 | 0 | 0.3 | 15 | 0 | 0 | 0 | 0 |
| | T | 0 | 0 | 0 | 0 | 0.1 | 0.9 | 0 | 0.2 | 0 | 0 |

When the orientation of the deaminase tethered to the nickase is reversed and the deaminase is tethered to the C-terminus, the primary editing outcome is C>G conversion at position C16 in target SGN000928. Very little C>T conversion is evident compared to the N-terminus configuration.

TABLE 19
C > N Editing Rate using deaminase
APG05840 and guide SGN000930

| | | SGN000930 | | |
|---|---|---|---|---|
| | | C17 | C19 | C22 |
| nAPG07433.1- | A | 0.1 | 0 | 0 |
| APG05840 | G | 17.5 | 0 | 0 |
| | T | 0.8 | 0 | 0 |

When the deaminase is tethered to the C-terminus of the nickase, the primary editing outcome is C>G conversion at position C17 in target SGN000930.

TABLE 20
C > N Editing Rate using deaminase
APG09980 and guide SGN000930

| | | SGN000930 | | |
|---|---|---|---|---|
| | | C17 | C19 | C22 |
| nAPG07433.1- | A | 0.2 | 0 | 0 |
| APG09980 | G | 13 | 0 | 0 |
| | T | 0.6 | 0 | 0 |

Using a second deaminase module, APG09980, the same trend is evident where when tethered to the C-terminus of the nickase, the predominant mutational outcome is C>G conversion at position C17.

TABLE 21
C > N Editing Rate using deaminase
APG05840 and guide SGN000930

| | | SGN000930 | | |
|---|---|---|---|---|
| | | C17 | C19 | C22 |
| APG05840- | A | 0.3 | 0.4 | 1.55 |
| nAPG07433.1 | G | 29.4 | 0.3 | 0.35 |
| | T | 2 | 1.85 | 3.2 |

When APG05840 is tethered to the N-terminus of nAPG07433.1, the primary mutation outcome is C>G conversion in position C17 with guide SGN000930.

TABLE 22
Overall mutation and deletion rate in base edited samples

| | | % of | % of |
|---|---|---|---|
| | | Mutated | Reads with |
| Construct | SGN | Reads | Deletions |
| APG09980- | SGN000930 | 21.42 | 2.09 |
| nAPG07433.1 | | | |
| nAPG07433.1- | SGN000930 | 18.38 | 0 |
| APG09980 | | | |
| APG09980- | SGN000930 | 21.505 | 0.313 |
| nAPG07433.1- | | | |
| USP2 | | | |
| APG05840- | SGN000930 | 18.735 | 0 |
| nAPG07433.1 | | | |
| APG05840- | SGN000930 | 22.595 | 0.355 |
| nAPG07433.1- | SGN000928 | 19.24 | 1.87 |
| USP2 | | | |
| nAPG07433.1- | SGN000930 | 17.475 | 0.285 |
| APG05840 | SGN000928 | 20.9 | 0.39 |

The data in this table is an average of multiple editing experiments. The percent of mutated reads is an estimate of the base editing rate in each sample. The percent of reads with deletions estimates the deletion rate in the sample. The C-terminus fusion of APG09980 to nAPG07433.1 has a lower deletion rate than the N-terminal fusion with and without a USP.

APG05840-nAPG07433.1-USP2 showed predominantly C>T conversion at positions C17 in guide SGN000930 and C16 and C21 in SGN000928. nAPG07433.1-APG09980 and nAPG07433.1-APG05840 showed predominantly C>G conversion at these same positions. All constructs showed editing in the same windows.

### Example 4: Targeted Base-Editing for Correction of Causal Disease Mutations

A database of clinical variants was obtained from NCBI ClinVar database, which is available through the world wide web at the NCBI ClinVar website. Pathogenic Single Nucleotide Polymorphisms (SNPs) were identified from this list. Using the genomic locus information, CRISPR targets in the region overlapping and surrounding each SNP were identified. A selection of SNPs that can be corrected using base editing in combination with an RGN, such as for example APG07433.1 or a variant thereof, to target the causal mutation is listed in Table 23. In Table 23 below, only one alias of each disease is listed. The “RS #” corresponds to the RS accession number through the SNP database at the NCBI website. The “Name” column contains the genetic locus identifier, the gene name, the location of the mutation in the gene, and the change resulting from the mutation.

TABLE 23
Disease Targets for Base Editing

| | | | Potential Target |
|---|---|---|---|
| | | | Sequence for |
| | | | RGN |
| | | | APG07433.1 |
| Indication | RS# | Name | (SEQ ID NO) |
| Acute neuronopathic | 1064651 | NM_000157.3(GBA): c.1342G > C | 122 |
| Gaucher's disease | | (p.Asp448His) | |
| Alpha-1-antitrypsin | 28931569 | NM_001127701.1(SERPINA1): c.194T > C | 123 |
| deficiency | | (p.Leu65Pro) | |
| Amyotrophic lateral | 121908287 | NM_014845.5(FIG. 4): c.122T > C | 124 |
| sclerosis type 11 | | (p.Ile41Thr) | |
| Ataxia- | 587781558 | NM_000051.3(ATM): c.2921 + 1G > A, | 125 |
| telangiectasia | | NM_000051.3(ATM): c.2921 + 1G > T, | |
| syndrome | | NM_000051.3(ATM): c.2921 + 1G > C | |
| Biotinidase | 28934601 | NM_000060.4(BTD): c.755A > G | 126 |
| deficiency | | (p.Asp252Gly) | |
| Carbohydrate- | 80338709 | NM_000303.2(PMM2): c.722G > C | 127 |
| deficient glycoprotein | | (p.Cys241Ser) | |
| syndrome type I | | | |
| Congenital myotonia | 80356696 | NM_000083.2(CLCN1): c.1655A > G | 128 |
| | | (p.Gln552Arg) | |
| Cowden syndrome 1 | 1114167621 | NM_000314.6(PTEN): c.210 − 1G > A, | 129 |
| | | NM_000314.6(PTEN): c.210 − 1G > C, | |
| | | NM_000314.6(PTEN): c.210 − 1G > T | |
| Cystic fibrosis | 75096551 | NM_000492.3(CFTR): c.2988 + 1G > A, | 130 |
| | | NM_000492.3(CFTR): c.2988 + 1G > C | |
| Dopamine beta | 74853476 | NM_000787.3(DBH): c.339 + 2T > C | 131 |
| hydroxylase | | | |
| deficiency | | | |
| Familial | 121908031 | NM_000527.4(LDLR): c.2043C > A (p.Cys681Ter), | 132 |
| hypercholesterolemia | | NM_000527.4(LDLR): c.2043C > G (p.Cys681Trp) | |
| Familial | 28940579 | NM_000243.2(MEFV): c.2177T > C (p.Val726Ala) | 133 |
| Mediterranean fever | | | |
| Glutaric aciduria | 199999619 | NM_000159.3(GCDH): c.1244 − 2A > C, | 134 |
| | | NM_000159.3(GCDH): c.1244 − 2A > G | |
| Inclusion body | 779694939 | NM_001128227.2(GNE): c.740T > C | 135 |
| myopathy 2 | | (p.Val247Ala) | |
| LCHAD Deficiency | 137852769 | NM_000182.4(HADHA): c.1528G > C | 136 |
| | | (p.Glu510Gln) | |
| Long QT syndrome | 267607277 | NM_006888.5(CALM1): c.293A > G | 137 |
| | | (p.Asn98Ser) | |
| Mucopolysaccharidos | 199801029 | NM_000203.4(IDUA): c.979G > C | 138 |
| is type I | | (p.Ala327Pro) | |
| Niemann-Pick | 80358259 | NM_000271.4(NPC1): c.3182T > C | 139 |
| disease | | (p.Ile1061Thr) | |
| Pendred syndrome | 111033313 | NM_000441.1(SLC26A4): c.919 − 2A > G | 140 |
| Primary familial | 727505017 | NM_002880.3(RAF1): c.769T > C | 141 |
| hypertrophic | | (p.Ser257Pro) | |
| cardiomyopathy | | | |
| Pyridoxine- | 121912707 | NM 001182.4(ALDH7A1): c. 1279G > C | 142 |
| dependent epilepsy | | (p.Glu427Gln) | |
| Shwachman | 113993993 | NM_016038.2(SBDS): c.258 + 2T > C | 143 |
| syndrome | | | |
| Wilson disease | 201738967 | NM_000053.3(ATP7B): c.122A > G | 144 |
| | | (p.Asn41Ser) | |

### Example 5: Demonstration of Gene Editing Activity in Plant Cells

Base-editing activity of an RGN-deaminase fusion protein of the invention is demonstrated in plant cells using protocols adapted from Li, et al., 2013 (Nat. Biotech. 31:688-691). Briefly, an expression vector comprising an expression cassette capable of expressing in plant cells an RGN-deaminase fusion protein operably linked to a SV40 nuclear localization signal (SEQ ID NO: 76) and a second expression cassette encoding a guide RNA targeting one or more sites in the plant PDS gene that flank an appropriate PAM sequence are introduced into Nicotiana benthamiana mesophyll protoplasts using PEG-mediated transformation. The transformed protoplasts are incubated in the dark for up to 36 hr. Genomic DNA is isolated from the protoplasts using a DNeasy Plant Mini Kit (Qiagen). The genomic region flanking the RGN target site is PCR amplified, products are purified, and the purified PCR products are analyzed using Next Generation Sequencing on Illumina MiSeq. Typically, 100,000 of 250 bp paired-end reads (2×100,000 reads) are generated per amplicon. The reads are analyzed using CRISPResso (Pinello, et al. 2016 Nature Biotech, 34:695-697) to calculate the rates of editing. Output alignments are analyzed for INDEL formation or introduction of specific cytosine mutations.

That which is claimed:

1. A polypeptide comprising an amino acid sequence selected from the group consisting of:
a) an amino acid sequence having at least 90% sequence identity to any one of SEQ ID NOs: 2 and 7-12; and
b) an amino acid sequence having at least 95% sequence identity to SEQ ID NO: 4 or 6;
wherein said polypeptide has deaminase activity.

2. The polypeptide of claim 1, comprising an amino acid sequence having 100% sequence identity to any one of SEQ ID NOs: 2, 4, and 6-12.

3. A nucleic acid molecule comprising a polynucleotide encoding a deaminase polypeptide, wherein the deaminase is encoded by a nucleotide sequence selected from the group consisting of:
a) a nucleotide sequence having at least 80% sequence identity to any one of SEQ ID NOs: 114-119;
b) a nucleotide sequence having at least 95% sequence identity to any one of SEQ ID NOs: 109, 111, and 113
c) a nucleotide sequence encoding an amino acid sequence having at least 90% sequence identity to any one of SEQ ID NOs: 2 and 7-12; and
d) a nucleotide sequence encoding an amino acid sequence having at least 95% sequence identity to SEQ ID NO: 4 or 6.

4. The nucleic acid molecule of claim 3, wherein the deaminase is encoded by a nucleotide sequence that has at least 90% sequence identity to any one of SEQ ID NOs: 114-119.

5. The nucleic acid molecule of claim 3, wherein the deaminase is encoded by a nucleotide sequence that has 100% sequence identity to any one of SEQ ID NOs: 109, 111, and 113-119.

6. The nucleic acid molecule of claim 3, wherein the deaminase polypeptide has an amino acid sequence having 100% sequence identity to any one of SEQ ID NOs: 2, 4, and 6-12.

7. The nucleic acid molecule of any one of claims 3-6, wherein said nucleic acid molecule further comprises a heterologous promoter operably linked to said polynucleotide.

8. A vector comprising said nucleic acid molecule of any one of claims 3-7.

9. A cell comprising said nucleic acid molecule of any one of claims 3-7 or said vector of claim 8.

10. A pharmaceutical composition comprising a pharmaceutically acceptable carrier and the polypeptide of claim 1 or 2, the nucleic acid molecule of any one of claims 3-7, the vector of claim 8, or the cell of claim 9.

11. A method for making a deaminase comprising culturing the cell of claim 9 under conditions in which the deaminase is expressed.

12. A method for making a deaminase comprising introducing into a cell the nucleic acid molecule of any of claims 3-7 or a vector of claim 8 and culturing the cell under conditions in which the deminase is expressed.

13. The method of claim 11 or 12, further comprising purifying said deaminase.

14. A fusion protein comprising a DNA-binding polypeptide and a deaminase having an amino acid sequence selected from the group consisting of:
a) an amino acid sequence having at least 90% sequence identity to any one of SEQ ID NOs: 2 and 7-12; and
b) an amino acid sequence having at least 95% sequence identity to SEQ ID NO: 4 or 6.

15. The fusion protein of claim 14, wherein said deaminase has 100% sequence identity to any one of SEQ ID NOs: 2, 4, and 6-12.

16. The fusion protein of claim 14 or 15, wherein the deaminase is a cytosine deaminase.

17. The fusion protein of any one of claims 14-16, wherein the DNA-binding polypeptide is a meganuclease, a zinc finger fusion protein, or a TALEN; or a variant of a meganuclease, a zinc finger fusion protein, or a TALEN, wherein the nuclease activity has been reduced or inhibited.

18. The fusion protein of any one of claims 14-16, wherein the DNA-binding polypeptide is an RNA-guided, DNA-binding polypeptide.

19. The fusion protein of claim 18, wherein the RNA-guided, DNA-binding polypeptide is an RNA-guided nuclease (RGN) polypeptide.

20. The fusion protein of claim 19, wherein the RGN is a Type II or Type V CRISPR-Cas polypeptide.

21. The fusion protein of claim 19 or 20, wherein the RGN is an RGN nickase.

22. The fusion protein of claim 21, wherein the RGN nickase has an inactive RuvC domain.

23. The fusion protein of claim 19 or 20, wherein the RGN is a nuclease-inactive RGN.

24. The fusion protein of claim 19, wherein the RGN has an amino acid sequence having at least 90% sequence identity to any one of the RGN sequences in Table 1.

25. The fusion protein of claim 19, wherein the RGN has an amino acid sequence of any one of the RGN sequences in Table 1.

26. The fusion protein of claim 19, wherein the RGN has an amino acid sequence having at least 90% sequence identity to any one of SEQ ID NOs: 74, 82, 87, 106, and 107.

27. The fusion protein of claim 19, wherein the RGN has an amino acid sequence of any one of SEQ ID NOs: 74, 82, 87, 106, and 107.

28. The fusion protein of claim 21, wherein the RGN nickase has an amino acid sequence having at least 90% sequence identity to any one of SEQ ID NOs: 75 and 88-98.

29. The fusion protein of claim 21, wherein the RGN nickase has an amino acid sequence having any one of SEQ ID NOs: 75 and 88-98.

30. The fusion protein of any of claims 14-29, wherein the fusion protein further comprises at least one nuclear localization signal (NLS).

31. The fusion protein of any one of claims 14-30, wherein said fusion protein further comprises a uracil stabilizing protein (USP).

32. The fusion protein of claim 31, wherein said USP has the sequence set forth as SEQ ID NO: 81.

33. The fusion protein of claim 14, wherein said fusion protein has an amino acid sequence of any one of SEQ ID NOs: 67, 68, 146, and 147.

34. A nucleic acid molecule comprising a polynucleotide encoding a fusion protein comprising a DNA-binding polypeptide and a deaminase, wherein the deaminase is encoded by a nucleotide sequence selected from the group consisting of:
a) a nucleotide sequence having at least 80% sequence identity to any one of SEQ ID NOs: 114-119;
b) a nucleotide sequence having at least 95% sequence identity to any one of SEQ ID NOs: 109, 111, and 113;
c) a nucleotide sequence encoding an amino acid sequence having at least 90% sequence identity to any one of SEQ ID NOs: 2 and 7-12; and
d) a nucleotide sequence encoding an amino acid sequence having at least 95% sequence identity to SEQ ID NO: 4 or 6.

35. The nucleic acid molecule of claim 34, wherein said deaminase is encoded by a nucleotide sequence has at least 90% sequence identity to any one of SEQ ID NOs: 114-119.

36. The nucleic acid molecule of claim 34, wherein said deaminase nucleotide sequence has 100% sequence identity to any one of SEQ ID NOs: 109, 111, and 113-119.

37. The nucleic acid molecule of claim 34, wherein said deaminase nucleotide sequence encodes an amino acid sequence having 100% sequence identity to any one of SEQ ID NOs: 2, 4, and 6-12.

38. The nucleic acid molecule of any one of claims 34-37, wherein the deaminase is a cytosine deaminase.

39. The nucleic acid molecule of any one of claims 34-38, wherein the DNA-binding polypeptide is a meganuclease, a zinc finger fusion protein, or a TALEN; or a variant of a meganuclease, a zinc finger fusion protein, or a TALEN, wherein the nuclease activity has been reduced or inhibited.

40. The nucleic acid molecule of any one of claims 34-38, wherein the DNA-binding polypeptide is an RNA-guided, DNA-binding polypeptide.

41. The nucleic acid molecule of claim 40, wherein the RNA-guided, DNA-binding polypeptide is an RNA-guided nuclease (RGN) polypeptide.

42. The nucleic acid molecule of claim 41, wherein the RGN is a Type II or Type V CRISPR-Cas polypeptide.

43. The nucleic acid molecule of claim 41 or 42, wherein the RGN is an RGN nickase.

44. The nucleic acid molecule of claim 43, wherein said RGN nickase has an inactive RuvC domain.

45. The nucleic acid molecule of claim 41 or 42, wherein the RGN is a nuclease-inactive RGN.

46. The nucleic acid molecule of claim 41, wherein the RGN has an amino acid sequence having at least 90% sequence identity to any one of the RGN sequences in Table 1.

47. The nucleic acid molecule of claim 41, wherein the RGN has an amino acid sequence of any one of the RGN sequences in Table 1.

48. The nucleic acid molecule of claim 41, wherein the RGN has an amino acid sequence having at least 90% sequence identity to any one of SEQ ID NOs: 74, 82, 87, 106, and 107.

49. The nucleic acid molecule of claim 41, wherein the RGN has an amino acid sequence of any one of SEQ ID NOs: 74, 82, 87, 106, and 107.

50. The nucleic acid molecule of claim 43, wherein the RGN nickase has an amino acid sequence having at least 90% sequence identity to any one of SEQ ID NOs: 75 and 88-98.

51. The nucleic acid molecule of claim 43, wherein the RGN nickase has an amino acid sequence having any one of SEQ ID NOs: 75 and 88-98.

52. The nucleic acid molecule of any of claims 34-51, wherein the polynucleotide encoding the fusion protein is operably linked at its 5′ end to a promoter.

53. The nucleic acid molecule of any of claims 34-52, wherein the polynucleotide encoding the fusion protein is operably linked at its 3′ end to a terminator.

54. The nucleic acid molecule of any of claims 34-53, wherein the fusion protein comprises one or more nuclear localization signals.

55. The nucleic acid molecule of any of claims 34-54, wherein the fusion protein is codon optimized for expression in a eukaryotic cell.

56. The nucleic acid molecule of any of claims 34-55, wherein the fusion protein is codon optimized for expression in a prokaryotic cell.

57. The nucleic acid molecule of any one of claims 34-56, wherein said fusion protein further comprises a uracil stabilizing protein (USP).

58. The nucleic acid molecule of claim 57, wherein said USP has the sequence set forth as SEQ ID NO: 81.

59. The nucleic acid molecule of claim 34, wherein said fusion protein has an amino acid sequence set forth as any one of SEQ ID NOs: 67, 68, 146, and 147.

60. A vector comprising the nucleic acid molecule of any one of claims 34-59.

61. The vector of claim 60, further comprising at least one nucleotide sequence encoding a guide RNA (gRNA) capable of hybridizing to a target sequence.

62. A cell comprising the fusion protein of any of claims 14-33.

63. The cell of claim 62, wherein the cell further comprises a guide RNA.

64. A cell comprising the nucleic acid molecule of any one of claims 34-59.

65. A cell comprising the vector of claims of claim 60 or 61.

66. A pharmaceutical composition comprising a pharmaceutically acceptable carrier and the fusion protein of any one of claims 14-33, the nucleic acid molecule of any one of claims 34-59, the vector of claim 60 or 61, or the cell of any one of claims 62-65.

67. A method for making a fusion protein comprising culturing the cell of any one of claims 62-65 under conditions in which the fusion protein is expressed.

68. A method for making a fusion protein comprising introducing into a cell the nucleic acid molecule of any of claims 34-59 or a vector of claim 60 or 61 and culturing the cell under conditions in which the fusion protein is expressed.

69. The method of claim 67 or 68, further comprising purifying said fusion protein.

70. A method for making an RGN fusion ribonucleoprotein complex, comprising introducing into a cell the nucleic acid molecule of any one of claims 34-59 and a nucleic acid molecule comprising an expression cassette encoding a guide RNA, or the vector of claim 60 or 61, and culturing the cell under conditions in which the fusion protein and the gRNA are expressed and form an RGN fusion ribonucleoprotein complex.

71. The method of claim 70, further comprising purifying said RGN fusion ribonucleoprotein complex.

72. A system for modifying a target DNA molecule comprising a target DNA sequence, said system comprising:
a) a fusion protein or a nucleotide sequence encoding said fusion protein, wherein said fusion protein comprises an RNA-guided nuclease polypeptide (RGN) and a deaminase, wherein the deaminase has an amino acid sequence selected from the group consisting of:
i) an amino acid sequence having at least 90% sequence identity to any one of SEQ ID NOs: 2 and 7-12; and
ii) an amino acid sequence having at least 95% sequence identity to SEQ ID NO: 4 or 6; and
b) one or more guide RNAs capable of hybridizing to said target DNA sequence or one or more nucleotide sequences encoding the one or more guide RNAs (gRNAs); and
wherein the one or more guide RNAs are capable of forming a complex with the fusion protein in order to direct said fusion protein to bind to said target DNA sequence and modify the target DNA molecule.

73. The system of claim 72, wherein said deaminase has an amino acid sequence having 100% sequence identity to any one of SEQ ID NOs: 2, 4, and 6-12.

74. The system of claim 72 or 73, wherein at least one of said nucleotide sequence encoding the one or more guide RNAs and said nucleotide sequence encoding the fusion protein is operably linked to a promoter.

75. The system of any one of claims 72-74, wherein the target DNA sequence is located adjacent to a protospacer adjacent motif (PAM) that is recognized by the RGN.

76. The system of any one of claims 72-75, wherein the target DNA molecule is within a cell.

77. The system of any one of claims 72-76, wherein the RGN of the fusion protein is a Type II or Type V CRISPR-Cas polypeptide.

78. The system of any one of claims 72-76, wherein the RGN of the fusion protein has an amino acid sequence having at least 90% sequence identity to any one of the RGN sequences in Table 1.

79. The system of any one of claims 72-76, wherein the RGN of the fusion protein has an amino acid sequence of any one of the RGN sequences in Table 1.

80. The system of any one of claims 72-76, wherein the RGN of the fusion protein has an amino acid sequence having at least 90% sequence identity to any one of SEQ ID NOs: 74, 82, 87, 106, and 107.

81. The system of any one of claims 72-76, wherein the RGN of the fusion protein has an amino acid sequence of any one of SEQ ID NOs: 74, 82, 87, 106, and 107.

82. The system of any one of claims 72-76, wherein the RGN of the fusion protein is an RGN nickase.

83. The system of claim 82, wherein the RGN nickase has an inactive RuvC domain.

84. The system of claim 82 or 83, wherein the RGN nickase has an amino acid sequence having at least 90% sequence identity to any one of SEQ ID NOs: 75 and 88-98.

85. The system of claim 82 or 83, wherein the RGN nickase is any one of SEQ ID NOs: 75 and 88-98.

86. The system of any one of claims 72-76, wherein the RGN of the fusion protein is a nuclease-inactive RGN.

87. The system of any of claims 72-86, wherein the fusion protein comprises one or more nuclear localization signals.

88. The system of any one of claims 72-87, wherein said fusion protein further comprises a uracil stabilizing protein (USP).

89. The system of claim 88, wherein said USP has the sequence set forth as SEQ ID NO: 81.

90. The system of claim 72, wherein the fusion protein has an amino acid sequence set forth as any one of SEQ ID NOs: 67, 68, 146, and 147.

91. The system of any of claims 72-90, wherein the fusion protein is codon optimized for expression in a eukaryotic cell.

92. The system of any of claims 72-91, wherein nucleotide sequences encoding the one or more guide RNAs and the nucleotide sequence encoding a fusion protein are located on one vector.

93. A ribonucleoprotein complex comprising said at least one guide RNA and said fusion protein of the system of any one of claims 72-92.

94. A cell comprising the system of any one of claims 72-92 or the ribonucleoprotein complex of claim 93.

95. A pharmaceutical composition comprising a pharmaceutically acceptable carrier and the system of any one of claims 72-92, the ribonucleoprotein complex of claim 93, or the cell of claim 94.

96. A method for modifying a target DNA molecule comprising a target DNA sequence, said method comprising delivering a system according to any one of claims 72-92 or a ribonucleoprotein complex of claim 93 to said target DNA molecule or a cell comprising the target DNA molecule.

97. The method of claim 96, wherein said modified target DNA molecule comprises a C>N mutation of at least one nucleotide within the target DNA molecule, wherein N is A, G, or T.

98. The method of claim 97, wherein said modified target DNA molecule comprises an C>T mutation of at least one nucleotide within the target DNA molecule.

99. The method of claim 97, wherein said modified target DNA molecule comprises an C>G mutation of at least one nucleotide within the target DNA molecule.

100. A method for modifying a target DNA molecule comprising a target sequence, said method comprising:
a) assembling an RGN-deaminase ribonucleotide complex in vitro by combining:
i) one or more guide RNAs capable of hybridizing to the target DNA sequence; and
ii) a fusion protein comprising an RNA-guided nuclease polypeptide (RGN), and at least one deaminase, wherein the deaminase has an amino acid sequence selected from the group consisting of:
I) an amino acid sequence having at least 90% sequence identity to any one of SEQ ID NOs: 2 and 7-12; and
II) an amino acid sequence having at least 95% sequence identity to SEQ ID NO: 4 or 6;
under conditions suitable for formation of the RGN-deaminase ribonucleotide complex; and
b) contacting said target DNA molecule or a cell comprising said target DNA molecule with the in vitro-assembled RGN-deaminase ribonucleotide complex;
wherein the one or more guide RNAs hybridize to the target DNA sequence, thereby directing said fusion protein to bind to said target DNA sequence and modification of the target DNA molecule occurs.

101. The method of claim 100, wherein said deaminase has an amino acid sequence having 100% sequence identity to any one of SEQ ID NOs: 2, 4, and 6-12.

102. The method of claim 100 or 101, wherein said modified target DNA molecule comprises a C>N mutation of at least one nucleotide within the target DNA molecule, wherein N is A, G, or T.

103. The method of claim 102, wherein said modified target DNA molecule comprises a C>T mutation of at least one nucleotide within the target DNA molecule.

104. The method of claim 102, wherein said modified target DNA molecule comprises a C>G mutation of at least one nucleotide within the target DNA molecule.

105. The method of any one of claims 100-104, wherein the RGN of the fusion protein is a Type II or Type V CRISPR-Cas polypeptide.

106. The method of any one of claims 100-104, wherein the RGN of the fusion protein has an amino acid sequence having at least 90% sequence identity to any one of the RGN sequences in Table 1.

107. The method of any one of claims 100-104, wherein the RGN of the fusion protein has an amino acid sequence of any one of the RGN sequences in Table 1.

108. The method of any one of claims 100-104, wherein the RGN of the fusion protein has an amino acid sequence having at least 90% sequence identity to any one of SEQ ID NOs: 74, 82, 87, 106, and 107.

109. The method of any one of claims 100-104, wherein the RGN of the fusion protein has an amino acid sequence of any one of SEQ ID NOs: 74, 82, 87, 106, and 107.

110. The method of any of claims 100-104, wherein the RGN of the fusion protein is an RGN nickase.

111. The method of claim 110, wherein said RGN nickase has an inactive RuvC domain.

112. The method of claim 110 or 111, wherein said RGN nickase has an amino acid sequence having at least 90% sequence identity to any one of SEQ ID NOs: 75 and 88-98.

113. The method of claim 110 or 111, wherein the RGN nickase is any one of SEQ ID NOs: 75 and 88-98.

114. The method of any one of claims 100-104, wherein the RGN of the fusion protein is a nuclease-inactive RGN.

115. The method of any of claims 100-114, wherein the fusion protein comprises one or more nuclear localization signals.

116. The method of any one of claims 100-115, wherein said fusion protein further comprises a uracil stabilizing protein (USP).

117. The method of claim 116, wherein said USP has the sequence set forth as SEQ ID NO: 81.

118. The method of claim 100, wherein said fusion protein has an amino acid sequence set forth as any one of SEQ ID NOs: 67, 68, 146, and 147.

119. The method of any of claims 100-118, wherein said target DNA sequence is located adjacent to a protospacer adjacent motif (PAM).

120. The method of any of claims 100-119, wherein the target DNA molecule is within a cell.

121. The method of claim 120, further comprising selecting a cell comprising said modified DNA molecule.

122. A cell comprising a modified target DNA sequence according to the method of claim 121.

123. A pharmaceutical composition comprising the cell of claim 122, and a pharmaceutically acceptable carrier.

124. A method for producing a genetically modified cell with a correction in a causal mutation for a genetically inherited disease, the method comprising introducing into the cell:
a) a fusion protein or a polynucleotide encoding said fusion protein, wherein said fusion protein comprises an RNA-guided nuclease polypeptide (RGN) and a deaminase, wherein the deaminase has an amino acid sequence selected from the group consisting of:
i) an amino acid sequence having at least 90% sequence identity to any one of SEQ ID NOs: 2 and 7-12; and
ii) an amino acid sequence having at least 95% sequence identity to SEQ ID NO: 4 or 6; and
b) one or more guide RNAs (gRNA) capable of hybridizing to a target DNA sequence, or a polynucleotide encoding said gRNA;
whereby the fusion protein and gRNA target to the genomic location of the causal mutation and modify the genomic sequence to remove the causal mutation.

125. The method of claim 124, wherein said deaminase has an amino acid sequence having 100% sequence identity to any one of SEQ ID NOs: 2, 4, and 6-12.

126. The method of claim 124 or 125, wherein the RGN of the fusion protein is a Type II or Type V CRISPR-Cas polypeptide.

127. The method of any one of claims 124-126, wherein the RGN of the fusion protein has an amino acid sequence having at least 90% sequence identity to any one of the RGN sequences in Table 1.

128. The method of any one of claims 124-126, wherein the RGN of the fusion protein has an amino acid sequence of any one of the RGN sequences in Table 1.

129. The method of any of claims 124-126, wherein the RGN of the fusion protein is an RGN nickase.

130. The method of claim 129, wherein said RGN nickase has an inactive RuvC domain.

131. The method of claim 129 or 130, wherein the RGN nickase has an amino acid sequence having at least 90% sequence identity to any one of SEQ ID NOs: 75 and 88-98.

132. The method of claim 129 or 130, wherein the RGN nickase is any one of SEQ ID NOs: 75 and 88-98.

133. The method of any one of claims 124-126, wherein the RGN of the fusion protein is a nuclease-inactive RGN.

134. The method of any of claims 124-133, wherein the fusion protein comprises one or more nuclear localization signals.

135. The method of any one of claims 124-134, wherein said fusion protein further comprises a uracil stabilizing protein (USP).

136. The method of claim 135, wherein said USP has the sequence set forth as SEQ ID NO: 81.

137. The method of claim 124, wherein said fusion protein has an amino acid sequence set forth as any one of SEQ ID NOs: 67, 68, 146, and 147.

138. The method of any one of claims 124-137, wherein the genome modification comprises introducing a C>T mutation of at least one nucleotide within the target DNA sequence.

139. The method of any one of claims 124-137, wherein the genome modification comprises introducing a C>G mutation of at least one nucleotide within the target DNA sequence.

140. The method of any of claims 124-139, wherein the cell is an animal cell.

141. The method of any one of claims 124-140, wherein the correction of the causal mutation comprises correcting a nonsense mutation.

142. The method of any one of claims 124-140, wherein the genetically inherited disease is a disease listed in Table 23.

143. A method for treating a disease, said method comprising administering to a subject in need thereof the fusion protein of any one of claims 14-33, the nucleic acid molecule of any one of claims 34-59, the vector of claim 60 or 61, the cell of any one of claims 62-65, 94, and 122, the system of any one of claims 72-92, the ribonucleoprotein complex of claim 93, or the pharmaceutical composition of any one of claims 66, 95, and 123.

144. The method of claim 143, wherein said disease is associated with a causal mutation and said pharmaceutical composition corrects said causal mutation.

145. The method of claim 143 or 144, wherein said disease is a disease a disease listed in Table 23.

146. Use of the fusion protein of any one of claims 14-33, the nucleic acid molecule of any one of claims 34-59, the vector of claim 60 or 61, the cell of any one of claims 62-65, 94, and 122, the system of any one of claims 72-92, or the ribonucleoprotein complex of claim 93 for the treatment of a disease in a subject.

147. The use of claim 146, wherein said disease is associated with a causal mutation and said treating comprises correcting said causal mutation.

148. The use of claim 146 or 147, wherein said disease is a disease listed in Table 23.

149. Use of the fusion protein of any one of claims 14-33, the nucleic acid molecule of any one of claims 34-59, the vector of claim 60 or 61, the cell of any one of claims 62-65, 94, and 122, the system of any one of claims 72-92, or the ribonucleoprotein complex of claim 93 for the manufacture of a medicament useful for treating a disease.

150. The use of claim 149, wherein said disease is associated with a causal mutation and an effective amount of said medicament corrects said causal mutation.

151. The use of claim 149 or 150, wherein said disease is a disease listed in Table 23.
